(19) United States
(12) Patent Application Publication
(10) Pub. No.: US 2025/0295413 A1
(43) Pub. Date: Sep. 25, 2025

(54) FLOW BALANCING DEVICES FOR BLOOD VESSELS

(71) Applicant: EDWARDS LIFESCIENCES CORPORATION, Irvine, CA (US)

(72) Inventors: Arvin T. Chang, Yorba Linda, CA (US); Scott Louis Pool, Laguna Hills, CA (US); Amir Yousef Sajjadi, Aliso Viejo, CA (US); Mazen Saadi Albaghdadi, Irvine, CA (US); Leonardo Paim Nicolau Da Costa, Tampa, FL (US); Yaeer E. Lev, Torrance, CA (US)

(73) Assignee: EDWARDS LIFESCIENCES CORPORATION, Irvine, CA (US)

(21) Appl. No.: 19/225,828

(22) Filed: Jun. 2, 2025

Related U.S. Application Data
(63) Continuation of application No. PCT/US2023/083864, filed on Dec. 13, 2023.
(60) Provisional application No. 63/387,922, filed on Dec. 16, 2022.
(60) Provisional application No. 63/502,915, filed on May 17, 2023.

(51) Int. Cl. A61B 17/12; A61B 17/00

(52) U.S. Cl. CPC A61B 17/12109; A61B 17/12031; A61B 17/12036; A61B 17/12172; A61B 2017/00367; A61B 2017/00734; A61B 2017/00876

(57) ABSTRACT

Systems and methods are described for modulating blood flow through a blood vessel. Devices may include one or more implantable devices comprising an expandable frame comprising a proximal end and a distal end and a longitudinal axis extending therethrough; a membrane comprising an inflow end and an outflow end, wherein the inflow end is at least partially installed within the distal end of the expandable frame; an actuator coupled to a control wire, the control wire being coupled to a portion of the membrane; a first magnet configured to induce rotation of the actuator to actuate the control wire to activate application of tension to the control wire and cause the membrane to radially collapse at the outflow end; and a second magnet configured to generate a changing magnetic field pole direction to cause rotation of the first magnet to cause the membrane to radially expand at the outflow end.

### CROSS-REFERENCE TO RELATED APPLICATIONS

This application is a continuation of International Application No. PCT/US23/83864, filed Dec. 13, 2023, which claims the benefit of U.S. Provisional Patent Application Ser. No. 63/387,922, filed Dec. 16, 2022; and U.S. Provisional Patent Application Ser. No. 63/502,915, filed May 17, 2023, the contents of each of which are herein incorporated by reference in their entireties.

### BACKGROUND

This disclosure relates generally to the field of medical devices and procedures, and more specifically to the field of blood flow management in blood vessels.

Chronic kidney disease (CKD) is a common comorbidity with many patients who suffer from chronic Heart Failure (HF). HF patients may also have elevated right atrium pressure, which may impair kidney function. In HF patients with elevated right atrium pressure, the kidneys may attempt to perform a diuresis process, but such a process may be difficult to perform efficiently due to the elevated pressure. For example, elevated right atrium pressure may hinder the ability of the kidneys to drive forward the flow of blood for accomplishing proper and efficient diuresis. Such unbalanced pressure coupled with the typical poor kidney efficiency of CKD patients may lead to an unending cycle of fluid overload for a person, which may result in an increase in congestion and heart failure admissions to the hospital.

### SUMMARY

Described herein are one or more methods and/or devices to facilitate management of blood flow through and/or into one or more blood vessels and/or chambers of a heart. There is a need for new and useful system and method for modulating blood flow through a blood vessel.

In some aspects, the techniques described herein relate to an implantable device for modulating blood flow through a blood vessel, the implantable device including: an expandable frame including a proximal end and a distal end and a longitudinal axis extending therethrough; and a membrane including an inflow end and an outflow end, wherein the inflow end is at least partially installed within the distal end of the expandable frame, wherein the membrane is configured to radially collapse at the outflow end and toward a central axis of the expandable frame, or radially expand away from the central axis of the expandable frame, in response to an actuation of a control wire coupled to a portion of the membrane, the central axis of the expandable frame being substantially parallel to the longitudinal axis.

In some aspects, the techniques described herein relate to a device for modulating blood flow through a blood vessel, the device being configured to be at least partially circumferentially disposed about an outer wall of the blood vessel, including: a ring having a control clement circumferentially disposed in the ring, wherein the control element has a first looped end and a second free end that passes through the first looped end, and wherein the control element is configured to translate relative to an inner diameter of the ring; and a mechanical actuator configured to be coupled to the second free end of the control element, wherein the mechanical actuator is configured to: apply tension to the second free end to reduce a diameter of the control element disposed in the ring; and reduce tension on the second free end to maintain or increase the diameter of the control element disposed in the ring.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates a bottom-up perspective view of an example flow modulating device for modulating blood flow through a blood vessel.

FIG. 1B illustrates a side view of the example flow modulating device of FIG. 1A.

FIG. 1C illustrates a side view of the example flow modulating device of FIG. 1A including one or more potential stasis zones.

FIG. 1D illustrates a side view of the example flow modulating device of FIG. 1A including one or more stasis reducing features.

FIG. 1E illustrates a side view of the example flow modulating device of FIG. 1A including one or more stasis reducing features.

FIG. IF illustrates a side view of the example flow modulating device of FIG. 1A including one or more stasis reducing features.

FIG. 1G illustrates a perspective view of an example membrane including one or more stasis reducing features for use with the flow modulating devices described herein.

FIG. 1H illustrates a bottom-up perspective view of the membrane of FIG. 1G.

FIG. 1I illustrates a bottom-up perspective view of an example membrane including one or more stasis reducing features for use with the flow modulating devices described herein.

FIG. 1J illustrates a bottom-up perspective view of another example membrane including one or more stasis reducing features for use with the flow modulating devices described herein.

FIG. 2A illustrates a side view of an example flow modulating device in an unrestricted blood flow state.

FIG. 2B illustrates a side view of the example flow modulating device in a restricted blood flow state.

FIG. 3A illustrates a side view of an example flow modulating device in an unrestricted blood flow state.

FIG. 3B illustrates an example flow modulating device with a substantially circular spring substantially circumferentially disposed about a blood vessel.

FIG. 3C illustrates an example flow modulating device with a shaped perimeter for substantially circumferentially surrounding a blood vessel.

FIG. 3D illustrates a side view of the example flow modulating device in a restricted blood flow state.

FIG. 4A illustrates a top-down perspective view of an example flow modulating device in an unrestricted blood flow state.

FIG. 4B illustrates a top-down perspective view of the example flow modulating device 400 in a restricted blood flow state.

FIG. 5A illustrates a side perspective view of an example flow modulating device in an unrestricted blood flow state.

FIG. 5B illustrates a side perspective view of the example flow modulating device in a restricted blood flow state.

FIG. 5C illustrates an example cross-sectional view of a lumen of the example flow modulating device in a constricted configuration.

FIG. 5D illustrates an example cross-sectional view of a lumen of the example flow modulating device in an unconstricted configuration.

FIG. 6A illustrates a top-down view of an example implantable device for modulating blood flow through a blood vessel.

FIG. 6B illustrates a top-down view of the example implantable device in an unrestricted blood flow state.

FIG. 7A illustrates a side view of an example flow modulating device for modulating blood flow through a blood vessel.

FIG. 7B illustrates a side view of an example flow modulating device in an unconstricted configuration.

FIG. 7C illustrates a side view of the example flow modulating device in a constricted configuration.

FIG. 8 illustrates a side view of an example flow modulating device for modulating blood flow through a blood vessel.

FIG. 9A illustrates a perspective view of an example device for modulating blood flow through a blood vessel.

FIG. 9B illustrates a perspective view of an example nozzle installed in the device for modulating blood flow through a blood vessel.

FIG. 9C illustrates a perspective view of an example nozzle installed in the device for modulating blood flow through a blood vessel.

FIG. 9D illustrates a perspective view of an example nozzle installed in the device or modulating blood flow through a blood vessel.

FIG. 10 illustrates a block diagram of an example system for modulating blood flow through one or more blood vessels.

FIG. 11A illustrates a schematic diagram of an example embodiment of a system 1100 for modulating blood flow through a blood vessel.

FIG. 11B illustrates a schematic diagram of an example embodiment of a system 1150 for modulating blood flow through a blood vessel.

FIG. 12 illustrates a flow diagram of an example process of modulating blood flow through one or more blood vessels.

FIG. 13 illustrates a schematic representation of portions of a human subject in which the devices described herein may be implanted.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel. The modulating and/or balancing of blood flow may be performed by the devices described herein to occlude, partially occlude, and/or otherwise manage or regulate blood flow to or through a portion of a blood vessel. In some examples, such modulation and/or balancing of blood flow to or through a blood vessel may result in additionally modulating pressure in the right atrium of the heart and/or other organs of the body.

The examples presented herein may relate to providing devices, methods, and/or methods of treatment (MOTs) for modulating, regulating and/or otherwise managing blood flow to or through one or more blood vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator). In some examples, the devices described herein may include blood flow management devices for reducing blood flow through a blood vessel, such as the Superior Vena Cave (SVC) and the Inferior Vena Cava (IVC), or related vessels. Managing blood flow through the SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying blood flow through the SVC and/or IVC, resulting in improved kidney perfusion and function.

In some examples, the devices, methods, and/or MOTs described herein may be utilized to solve a technical problem of unwanted pressure increases in the right atrium in patients that have chronic kidney disease (CKD) and/or heart failure (HF). For example, patients with CKD and/or HF may exhibit reduced kidney function when pressure in the right atrium of the heart is above a predefined pressure threshold. The predefined pressure threshold may be used as a basis to determine whether a patient is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrium pressure increases and/or pressure variations. In particular, the devices, methods, and/or MOTs described herein can be used to reduce and maintain low pressure in the right atrium, which provides a technical effect of enabling the kidneys to more effectively filter blood.

In addition, the devices, methods, and/or MOTs described herein can solve a further technical problem of accumulation of blood in the venous system. For example, the devices described herein may be used to reduce the accumulation of blood in the venous system, which can provide an advantage and technical effect of ensuring that pressure is not increased in the SVC and/or the IVC. Such devices can advantageously eliminate excessive hospital readmissions and/or can provide for a long-term blood flow management therapy, improving both quality of life and overall survival rates and with a lower cost to a healthcare system. Furthermore, the devices, methods, and/or MOTs described herein can be used to solve a further technical problem of regulating (e.g., modulating) blood flow return, thus further mitigating pressure build-up in the right atrium. The examples described herein can perform blood flow management actively and/or passively to assist in reducing and/or maintaining right atrium pressures to a relatively low pressure even when a surge in blood volume occurs in one or more vessels of the venous system.

### SYSTEMS AND DEVICES

Disclosed herein are systems and methods for modulating blood flow through a blood vessel. In some examples, the implantable flow modulating devices described herein may be used in blood flow occlusion therapy. For example, the devices described herein may relate to venous occlusion therapy using implantable and/or electronically controlled flow restricting devices for the treatment of acute heart failure. Some devices may be non-implantable or partially implantable. In some examples, the devices described herein generally function to occlude or partially occlude a blood vessel, such as the SVC or the IVC. In some examples, the devices described herein have been contemplated for use in a patient/user having chronic heart failure and/or chronic kidney disease, but may be used in any vessel needing flow regulation therethrough.

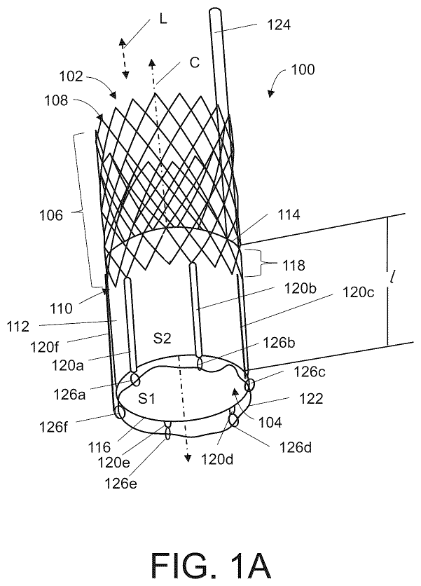

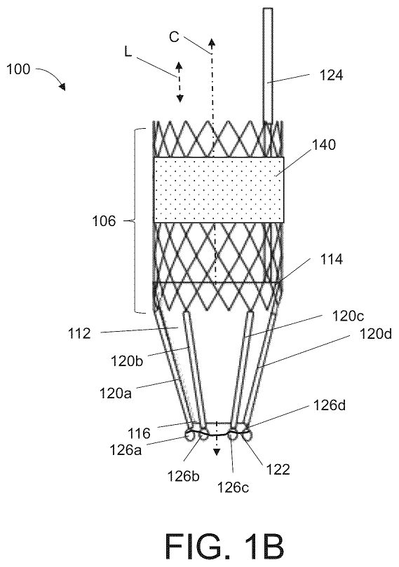

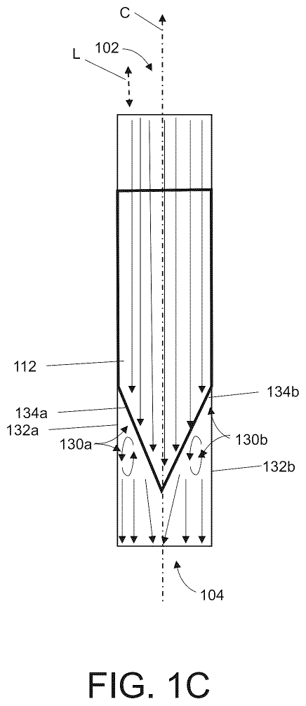

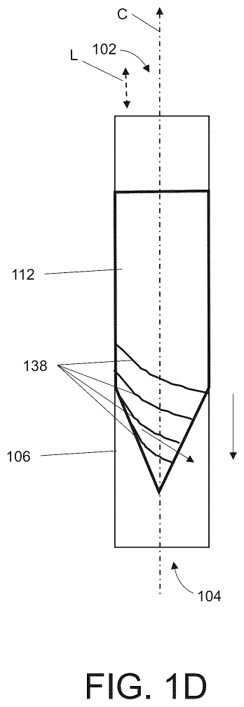

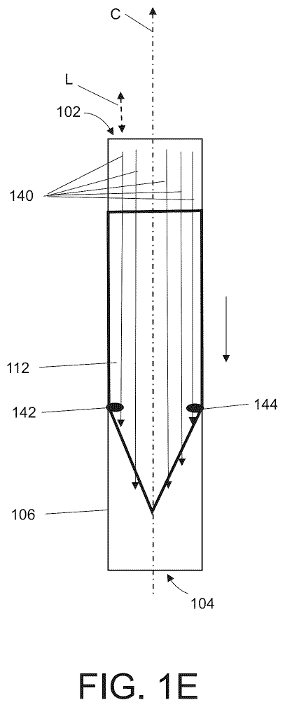

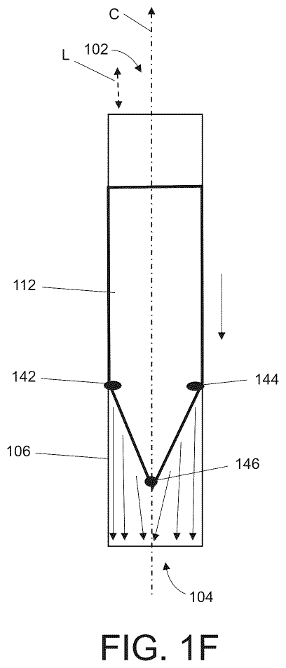

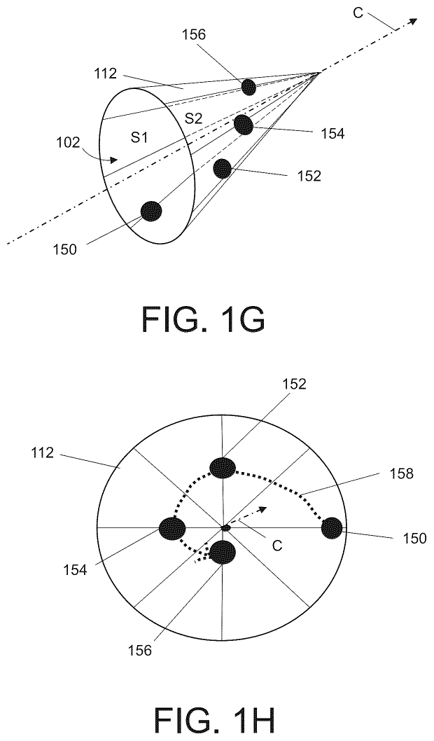

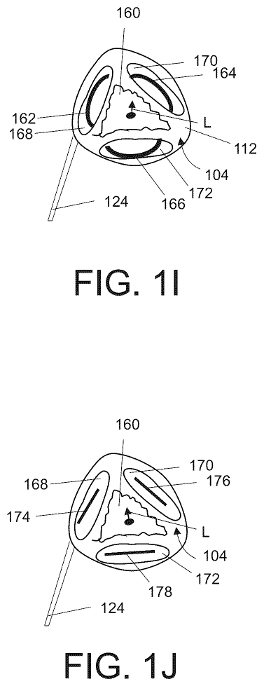

FIGS. 1A-1B illustrate views of an example flow modulating device 100 for modulating blood flow through a blood vessel. The device 100 may be implanted into a blood vessel, such as the superior vena cava, the inferior vena cava, or any other blood vessel where modulating blood flow is desired. For example, the device 100 may modulate a volume of blood flowing from the superior vena cava into a right atrium to decrease right atrial pressure.

At a high level, the device 100 may include a self-expanding or balloon expandable frame (e.g., stent) that may be delivered into the blood vessel (e.g., via jugular access, subclavian access, or transfemoral access) using a sheathed catheter (not shown). The frame may include or be coupled to a membrane that is further coupled to a flexible control wire threaded through a portion (e.g., end portion) of the membrane. The flexible control wire may function as a lasso to be actuated by an actuation device to radially expand and constrict (uniformly or nonuniformly) a perimeter of the end portion of the membrane to function as an adjustable blood flow restrictor.

FIG. 1A illustrates a bottom-up perspective view of an example flow modulating device 100 for modulating blood flow through a blood vessel. In this example, the device 100 is shown in an unrestricted blood flow state. The unrestricted blood flow state may represent a state of device 100 in which both an inflow end 102 (e.g., an inflow) and an outflow end 104 (an outflow) are open to receive fluid (e.g., blood, drugs, saline, etc.). The fluid flows through the inflow end 102 and through the device 100 to the outflow end 104 at least partially along an inner surface S1 opposite an outer surface S2 and through an opening defined by the device 100. For example, the device 100 may be in the expanded state when both the inflow end 102 and the outflow end 104 are open to receive fluid (e.g., blood, drugs, saline, etc.) therethrough when the device 100 is implanted in a blood vessel.

The device 100 includes an expandable frame 106 that includes a proximal end 108 and a distal end 110, and a longitudinal axis (L) extending therethrough. The proximal end 108 may correspond to the inflow end 102 of the device 100. The frame 106 may be a stent, for example constructed of metal wire (e.g., stainless steel, platinum, Nitinol® wire or another shape memory alloy), or other material suitable for implantation in the human body. In some examples, the expandable frame 106 is a bare metal stent, such that the expandable frame 206 is configured to be at least partially incorporated into an inner wall of the blood vessel. In some examples, the expandable frame 106 has a pro-endothelialization coating, such that the expandable frame can be at least partially incorporated into an inner wall of the blood vessel. This incorporation may allow a site of the device 100 to maintain a non-thrombogenic, non-immunogenic environment with respect to the device 100. For example, the coating of the frame 106 may be any pro-endothelial factor including, but not limited to, endothelial growth factor, vascular endothelial growth factor, or any related compound.

The device 100 also includes a membrane 112 with an inflow end 114 and an outflow end 116. The inflow end 114 is shown at least partially installed within the distal end 110 of the expandable frame. For example, the membrane 112 is coupled to an inner surface portion of the expandable frame 106, as shown by an overlap 118. The membrane 112 may be installed within (and overlapping) about 25% of a length of the frame 106. In some examples, the membrane 112 may be installed within (and overlapping) about 10% to about 50% of the length of the frame 106. In some examples, the membrane 112 may be installed within (and overlapping) about 10% to about 25% of the length of the frame 106. In some examples, the membrane 112 may be installed within (and overlapping) about 20% to about 30% of the length of the frame 106. In some examples, the inflow end 114 of the membrane 112 is decoupled from the distal end 110 of the frame 106 and without an overlap. For example, the inflow end 114 may be reversibly coupled to the distal end 110 of the frame 106. The membrane 112 may be formed of a polymer a copolymer, a textile (e.g., woven, knitted, nonwoven, or braided), a tissue (e.g., bovine pericardium, equine pericardium, porcine vena cava, etc.), or a combination thereof.

In some examples, the membrane 112 is substantially tubular-shaped with a substantially circular cross section about a central axis (C). In some examples, the membrane 112 may be substantially elliptical in shape with a substantially elliptical cross section about the central axis (C). In some examples, the membrane 112 may be substantially flexible such that the shape may take on an irregular perimeter that may form a shape of the blood vessel in which the device 100 is installed, for example, when blood flow is provided from the inflow end 114 through to the outflow end 116.

In some examples, the membrane 112 is adjustable to any number of positions between expanded and collapsed. For example, the outflow end 116 may collapse inward toward the central axis (C) associated with the frame 106 and at any interval between fully expanded and fully contracted. The outflow end 116 may also open or expand outward away from the central axis (C) associated with the frame 106. In some examples, the membrane 112 may be expanded or contracted from a particular device state into an expanded position, a partially expanded position, or a collapsed position. For example, when the device 100 is in the expanded position, the device 100 may be caused to be configured into a partially expanded position or a collapsed position by partially or fully collapsing, respectively, the outflow end 116 of the membrane 112 toward the central axis (C). When the device 100 is in the collapsed position, the device 100 may be caused to be configured into a partially expanded position or an expanded position by partially or fully expanding, respectively, the outflow end 116 of the membrane 112 toward the central axis (C).

The expanded position of the membrane 112 may allow the blood flow through the blood vessel. For example, when the device 100 is implanted in a blood vessel and is configured in the expanded position, the device 100 may allow blood to flow from the inflow end 114 through to the outflow end 116, without substantially hindering the blood flow speed or the blood flow amount.

The partially expanded position of the membrane 112 may allow partial occlusion of the blood vessel. For example, when the device 100 is implanted in a blood vessel and is configured in the partially expanded position, the device 100 may allow a partial amount of blood to flow from the inflow end 114 through to the outflow end 116 and may hinder a flow of the blood flow by a predefined amount associated with a cross sectional area formed when the outflow end 116 is partially closed (e.g., partially collapsed, partially expanded).

The collapsed position of the membrane 112 may occlude the blood vessel. In some embodiments, the occlusion of the blood vessel is a full occlusion. In some embodiments, the occlusion of the blood vessel is a partial occlusion.

As shown in FIG. 1A, the outflow end 116 of the membrane 112 is coupled to a plurality of elongate support members 120a, 120b, 120c, 120d, 120c, and 120f. The elongate support members 120a-120f may be flexible to allow the membrane 112 to bend radially toward the central axis (C) of the frame 106 at the outflow end of the membrane 112 when the control wire is actuated. In some examples, the elongate support members 120a-120f may be flexible to allow the membrane 112 to bend and/or collapse at the outflow end in an asymmetrical collapse or tilt toward one or more portions of a circumferential edge of the outflow end of the membrane. For example, rather than radially collapsing in a symmetrical fashion toward the central axis (C), the membrane may be supported by zero, one, or more elongate support members that allow the membrane to asymmetrically reduce the circumference (e.g., perimeter) of the outflow end of the membrane, which at least partially occludes or slows flow at the outflow end.

The plurality of support members 120a-120f may be arranged radially around the membrane 112. For example, the plurality of support members 120a-120f may be arranged radially around an outer surface or an inner surface of the membrane 112. For example, the plurality of elongate support members 120a-120f may be equidistantly arranged radially around a surface of the membrane 112. In some examples, the plurality of elongate support members 120a-120f may be arranged non-equidistantly around a surface of the membrane 112. In some examples, the plurality of elongate support members 120a-120f may be arranged radially around a surface of the membrane 112 such that support members 120a-120c are arranged around a first semi-circular and surface portion of the membrane 112 while support members 120d-120f are arranged around a second semi-circular portion of the membrane. For example, the support members 120a, 102b, and 120c may be separated by substantially similar distance apart around the first semi-circular and surface portion of the membrane 112 and the support members 120d, 102e, and 120f may be separated by substantially equidistant apart around the second semi-circular and surface portion of the membrane 112. In such an arrangement, the support member 120c may be arranged substantially adjacent to support member 120d, but may be arranged at a closer distance than the distance between support member 120a and 120b or between support member 120b and support member 120c. Similarly, the support member 120a may be arranged adjacent to support member 120f, but may be arranged at a closer distance than the distance between support member 120c and 120d or between support member 120c and support member 120c.

In some examples, the plurality of elongate support members 120a-120f may extend from the outflow end 116 and toward the inflow end 114 running substantially parallel to the longitudinal axis (L) of the device 100. The support members 120a-120f may extend a portion of a length (l) of the membrane 112. For example, the support members 120a-120f may extend across about 50% to about 90% of an outer surface of the membrane 112. In some examples, the support members 120a-120f may extend a full length (l) of the membrane 112. The length of the membrane 112 may be about 5 millimeters to about 5 centimeters. The radius of the membrane 112 may be about 10 millimeters to about 30 millimeters. The thickness of the membrane 112 may be about 0.01 millimeters to about 1 millimeter.

Although the device 100 includes six support members 120a-120f, more or fewer support members are possible. For example, the device 100 may have zero or one support member; one to two support members; two to three support members; three to five support members; four to six support members; five to seven support members; or five to eight support members.

As shown in FIG. 1A, the device 100 further includes an eyelet 126a, an eyelet 126b, an eyelet 126c, an eyelet 126d, an eyelet 126e, and an eyelet 126f. The eyelets 126a-126f may be coupled to respective support members 120a-120f. For example, the eyelet 126a is coupled to a distal end of the support member 120a; the eyelet 126b is coupled to a distal end of the support member 120b; the eyelet 126c is coupled to a distal end of the support member 120c; the eyelet 126d is coupled to a distal end of the support member 120d; the eyelet 126e is coupled to a distal end of the support member 120e; the eyelet 126f is coupled to a distal end of the support member 120f. Each eyelet 126a-126f may be configured to receive a portion of the control wire 122 threaded therethrough. In some examples, the control wire 122 may be threaded through two or more of the eyelets 126a-126f (e.g., fewer than all) to partially or fully close the outflow end 116 of the membrane 112 in an asymmetrical collapse, partial closure, full closure of the outflow end 116. The eyelets 126a-126f extend beyond the distal end of each respective support member 120a-120c. Each eyelet 126a-126f is formed as an aperture having a substantially annular opening. The aperture of each respective eyelet 126a-126e is arranged to receive the control wire 122 when threaded therethrough such that when the control wire 122 is actuated, the eyelets 126a-126f move radially (e.g., cinching each eyelet together) toward the central axis (C) of the expandable frame 106. For example, actuating the control wire 122 reversibly cinches the membrane 112 toward the central axis (C) by bringing the eyelets 126a-126f together at the outflow end 116 of the membrane 112 to occlude or partially occlude a blood vessel in which the device 100 is implanted.

In some examples, the eyelets 126a-126f may instead be apertures shaped as a circle, a semi-circle, a slit, a loop, a ring, an oval, etc., and/or another shape that allows a threading member to be received in the shape. For example, the eyelets 126a-126f may be punched or riveted rings or suture loops, any or all of which may be installed in the membrane or on the elongate support members 120a-120f.

Referring again to FIG. 1A, the outflow end 116 of the membrane 112 may correspond to the outflow end 104 of device 100. The outflow end 116 of the membrane 112 may be triggered to radially collapse toward the central axis (C) associated with the frame 106. For example, the outflow end 116 of the membrane 112 may be configured to radially collapse inward at the outflow end 116 by moving the plurality of support members 120a-120f and attached eyelets 126a-126f toward the central axis (C) or radially collapse outward at the outflow end 116 by moving the plurality of support members 120a-120f and attached eyelets 126a-126f away from the central axis (C). The radial collapse or expansion may occur in response to an actuation of a control wire 122. The control wire 122 may be coupled to a portion of the membrane 112 or at least one of the elongate support members 120a-120f to trigger the expansion or the collapse.

FIG. 1B illustrates a side view of the example flow modulating device of FIG. 1A. In this example, the device 100 is shown with the membrane 112 in a partially collapsed state. The partially collapsed state may represent a restricted blood flow state in which the membrane 112 radially collapses toward the central axis (C) of the frame 106 to reduce (or stop) blood flow through the blood vessel. Such a state may allow for a partial flow of blood, for example, through a lumen associated with the membrane 112. FIG. 1A, by contrast depicts the device 100 in an unrestricted blood flow state in which the membrane 112 is depicted radially expanded away from the central axis (C) of the expandable frame 106 to allow blood to flow through the blood vessel in which device 100 is implanted.

Positioning the membrane 112 in the partially collapsed state (e.g., a restricted blood flow state) shown in FIG. 1B, the control wire 122 may be actuated by a control element 124 coupled to, or otherwise in communication with, the control wire 122 to cause tensioning of the control wire 122 and closure or partial closure (e.g., cinching) of the membrane 112 at the outflow end 116. For example, the membrane 112 may be adjustable to form a cinched portion at the outflow end 116. For example, the cinching to form a cinched portion may include causing a perimeter of the outflow end 116 to be pleated, folded, or otherwise collapsed toward the central axis (C) by tensioning the control wire 122, which may result in reversibly reducing or closing the cross section at the outflow end 116. Such cinching of the perimeter of the outflow end 116 may be performed by device 100 to fully collapse the outflow end 116 of the membrane 112 resulting in occlusion of the blood vessel associated with the device 100. The cinching of the perimeter of the outflow end 116 may also be performed by device 100 to partially collapse the outflow end 116 of the membrane 112 resulting in a partial occlusion of the blood vessel associated with the device 100.

In general, actuating the control wire 122 may result in positioning the membrane 112 and/or device 100 in an unrestricted blood flow state or a restricted blood flow state. The device 100 may include an actuation device (not shown), either active or passive, to actuate the control wire 122. The actuation device may use a power source associated with or coupled to device 100 to induce changes in blood flow states of the membrane 112 and/or other portion of device 100. In some examples, the actuation device may use passively induced movement. For example, passively moving a portion of the device 100 may include manually actuating pull wires (e.g., sutures, actuation wires/cords/elements, etc.) and/or anatomy responses (e.g., changes in vessel inner diameter, intra-vessel pressure, etc.

In some examples, the actuation device for actuating the control wire of the device 100 may include an actuator coupled to the control wire 122 of the device 100, a first magnet to induce rotation of the actuator, and a control device communicatively coupled to the actuator. In some examples, the first magnet is a permanent magnet, and the second magnet is a permanent magnet. In some examples, the first magnet is a permanent magnet, and the second magnet is an electromagnet. In some examples, the actuation device may be a magnetically driven actuator. In such an example, the control device may include a second magnet for generating a changing magnetic field pole direction to cause rotation of the first magnet and operation of the control wire 122 and device movement to the unrestricted blood flow state or to the restricted blood flow state. For example, the actuation device may cause rotation of the second magnet in a first direction to induce rotation of the first magnet, thereby causing the actuator to tension the control wire 122 to cause the membrane 112 to radially collapse inward and toward the central axis (C) at the outflow end 116. For example, the first direction of rotation of the second magnet may attract the first magnet.

The actuation device may also cause rotation of the second magnet in a second direction to induce rotation of the first magnet, thereby causing the actuator to release tension in the control wire 122 to cause the membrane 112 to radially open at the outflow end 116. For example, the second direction of rotation of the second magnet may repel the first magnet.

In some examples, the control device is implanted in the same user (e.g., subject) that the device 100 is implanted within. The control device may be implanted adjacent to the device 100 or remote from the device 100. In some examples, the control device is implanted subcutaneously in the subject. In some examples, the control device is disposed external to a body of a subject (e.g., a user) associated with the device.

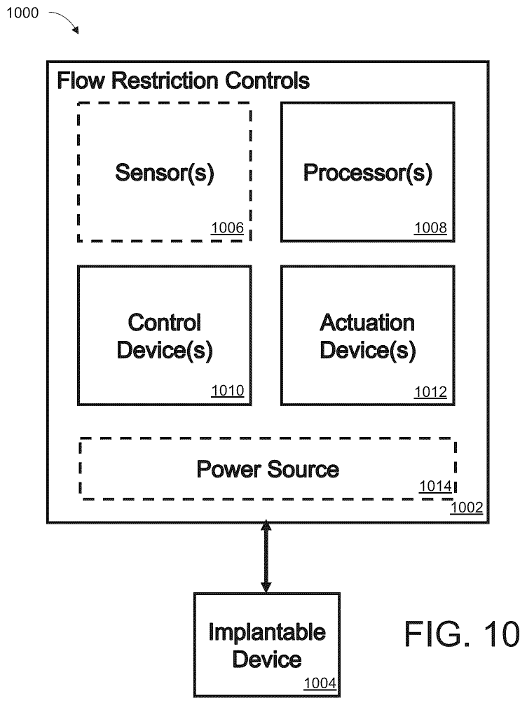

In some examples, the device 100 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to an actuator (e.g., actuation device 1012) associated with device 100, the microprocessor 1008, and the sensor 1006. For example, the sensor 1006 may sense characteristics of blood flow in the blood vessel (e.g., blood pressure) and may cause the microprocessor to provide signals to the actuator (e.g., actuation device 1012) and/or control clement 124 and/or control wire 122 (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to tension the control devices 1010, or release tension in the control devices 1010 based on the sensed pressure in the blood vessel.

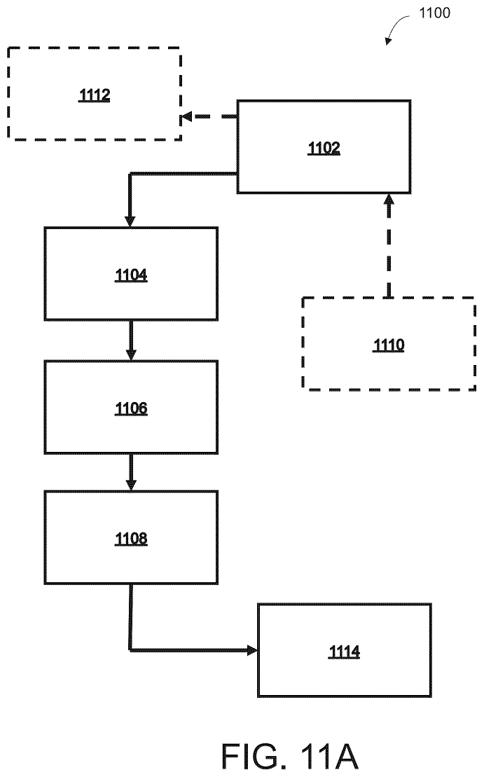

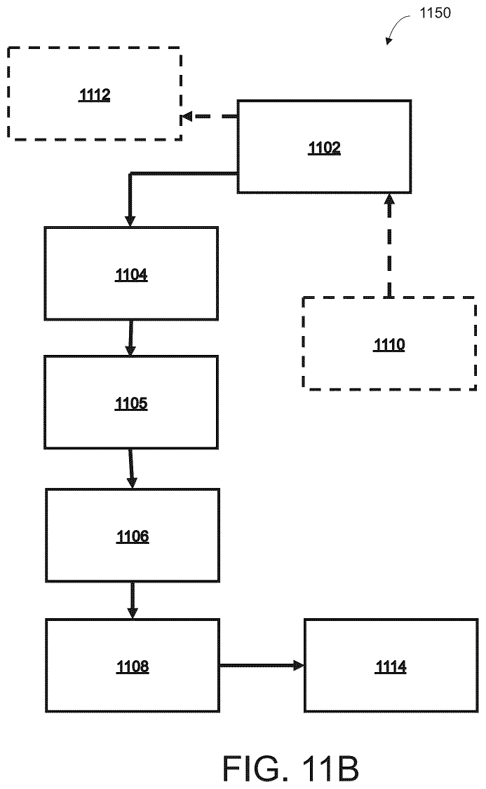

In some examples, the sensor 1006 may be communicatively coupled to device 100. The sensor 1006 may include one or more of an image sensor, a strain gauge, a piezoelectric sensor, a capacitance sensor, and/or a vacuum pressure sensor. If the device 100 is coupled to a power source (e.g., power source 1014), the power source may include an induction coil. The induction coil may be used to operate one or more of such magnets, as described in further detail in FIGS. 11A-11B.

In operation, the device 100 may receive a signal from an actuator that triggers the control element 124 to cause actuation of the control wire 122 and in turn causes a radial collapse of the membrane 112 at the outflow end 116. Such an actuation of the control wire 122 may cause the control wire 122 to be tensioned and to pull the eyelets radially toward the central axis (C) to collapse or partially collapse the membrane 112. In addition, the outflow end 116 of the membrane 112 is configured to radially expand away from the central axis (C) of the expandable frame 106, in response to an actuation of the control wire 122. The control wire 122 may be actuated by the control element 124 connected to the control wire 122 in a similar fashion as described above to cause the control wire 122 to release the tension and to release the eyelets radially away from the central axis (C) to expand or partially expand the membrane 112.

The device 100 may include a power source (not shown) coupled to the control wire 122. The power source may include a battery or a wall outlet that may be electrically connected to the control wire 122 or another portion of device 100. The electrical connection may allow active powering of device 100 operations. In such an example, a processor may be utilized to send and/or receive signals to activate device operations via the actuation device. In some examples, the actuation device can be configured to send a first signal to the control wire 122 to activate application of tension to the control wire 122. For example, a processor (not shown) may be programmed to trigger tensioning of the control wire 122 in response to detecting a particular condition of the blood vessel or the device 100. The tensioning of the control wire 122 may result in cinching the outflow end 116 of the membrane to place the device in a restrictive blood flow state. Similarly, the actuation device can be configured to send a second signal to the control wire to activate releasing of the tension from the control wire 122 in response to detecting another condition of the blood vessel or the device 100. For example, a processor (not shown) may be programmed to trigger a release of tension in the wire 122 in response to detecting a particular condition of the blood vessel or the device 100. The release of the tension of the control wire 122 may result in uncinching the outflow end 116 of the membrane to place the device in an unrestrictive blood flow state.

In some examples, the device 100 may include the expandable frame 106 that includes a proximal end 108 and a distal end 110, and a longitudinal axis (L) extending therethrough. Such a device may also include a membrane 112 with an inflow end 114 and an outflow end 116. The inflow end 114 may be at least partially installed within the distal end 110 of the expandable frame 106. In some examples, the membrane 112 is substantially tubular-shaped with a substantially circular cross section about a central axis (C). In some examples, the membrane 112 may be substantially elliptical in shape with a substantially elliptical cross section about the central axis (C). In some examples, the membrane 112 may be substantially flexible such that the shape may take on an irregular perimeter that may form a shape of the blood vessel in which the device 100 is installed, for example, when blood flow is provided from the inflow end 114 through to the outflow end 116.

In operation, the membrane 112 may radially collapse at the outflow end 116 and toward a central axis (C) of the expandable frame 106, or radially expand away from the central axis (C) of the expandable frame 106, in response to an actuation of a control wire 122 coupled to a portion of the membrane, as described elsewhere herein. In some examples, the membrane 112 may collapse at the outflow end in an asymmetrical collapse or tilt toward one or more portions of a circumferential edge (e.g., perimeter) of the outflow end 116. In some examples, the collapsing is supported by one or more elongate support members coupled to the outflow end 116 of the membrane 112. However, the elongate support members 120a-120f may be absent in some embodiments of device 100 and in such embodiments, two or more eyelets (e.g., eyelets 126a-126f) may support expanding and collapsing of the outflow end 116 of the membrane 112.

In some examples, actuating the control wire 122 results in arranging the membrane in an unrestricted blood flow state or a restricted blood flow state, as described elsewhere herein. In general, the membrane 112 may be adjustable to a plurality of positions between expanded and collapsed, including, but not limited to an expanded position configured to allow the blood flow through the blood vessel, a partially expanded position configured to partially occlude the blood vessel, and a collapsed position configured to block the outflow end to occlude the blood vessel.

In some examples, each respective eyelet in the plurality of eyelets 126a-126f may be attached or installed within a distal end of a respective elongate support or installed within the membrane 112. In some examples, the eyelets 126a-126f each include an aperture and arranged to receive the control wire 122 when threaded therethrough such that when the control wire 122 is actuated, one or more eyelets 126a-126f may move radially toward the central axis of the expandable frame 106.

In some examples, the implantable device 100 includes a skirt membrane 140 wrapped around at least a portion of an exterior surface of the frame 106. The skirt membrane 140 may function to reduce blood stasis and/or pooling around the implantable device 100. For example, the skirt membrane 140 may be at least partially incorporated into an inner wall of the blood vessel to reduce or eliminate clotting and/or blood stasis within cells and struts of the device 100 by providing an effective seal between the skirt membrane 140 and the blood vessel wall.

In some examples, the skirt membrane 140 may be formed of poly-delta-valerolactone (PVL). In some examples, the skirt membrane 140 may be formed of PVL and another polymer. In some examples, the skirt membrane 140 may be formed of mesh or braided metal that may be coated. In some examples, the skirt membrane 140 may be formed of a textile.

In some embodiments, when a flow modulating device is in a partially or substantially fully closed, occluded, or restricted state (e.g., one or more membranes 112 are partially or fully expanded at the outflow end 104), blood may pool, exhibit stasis, or create eddies at or proximal to an upstream or inflow end 102 and/or at a downstream or outflow end 104 of the flow modulating device (e.g., flow modulating device 100). This pooling, stopping, or slowing of blood flow may create one or more stasis zones within the IVC, SVC, or peripheral vessel. For example, these stasis zones may be created where the membrane 112 couples to the frame 106; where the membrane 112 and frame 106 together define a pocket, groove, indentation, or concave section; where the membrane 112 contacts a support member 120a-120f; and the like. To alleviate blood stasis within a potential stasis zone, a flow modulating device may include one or more stasis reduction solutions. For example, a flow modulating device 100 may include the membrane 112, as shown in FIG. IC, that may include one or more potential stasis zones 130a, 130b positioned between a vessel wall 132a, 132b and an outside surface 134a, 134b of the membrane 112.

To alleviate blood stasis and/or pooling, stasis reducing (or stasis mitigating) features may be included on membrane 112 to ensure that blood may flow through the one or more of the potential stasis zones 130a, 130b. For example, FIG. 1D illustrates a side view of the example flow modulating device of FIG. 1A including one or more stasis reducing features. The stasis reducing features in this example include a plurality of grooves 138 that may function as conduits (e.g., gutters, paths, etc.) to move blood along a surface of the membrane 112 and/or the frame 106. The grooves 138 are shown on the membrane 112 in a spiral shape along the outer surface of a portion of the membrane. One skilled in the art will appreciate that more or fewer grooves may be provided on membrane 112 and such grooves may be angled at any angle to promote blood flow from a particular stasis zone. That is, the grooves 138 may be configured at an angle to enable blood to flow along portions of the expandable frame 106 and/or membrane 112 from the inflow end 102 to the outflow end 104 to reduce blood stasis around the implantable device having the grooves 138 therein. The grooves 138 may define a stasis reducing flow path that extends substantially spirally or substantially helically around central axis (C) and along at least a portion of the outer surface of the membrane 112.

FIG. 1E illustrates a side view of the example flow modulating device of FIG. 1A including one or more stasis reducing features. For example, a flow modulating device 100 may include the membrane 112, as shown in FIG. 1E. Similar to FIG. 1C, the embodiment of FIG. 1E may include one or more potential stasis zones 130a, 130b (FIG. 1C) positioned between a vessel wall 132a, 132b (FIG. 1C) and an outside surface 134a, 134b (FIG. 1C) of the membrane 112. To alleviate blood stasis and/or pooling, stasis reducing (or stasis mitigating) features may be included on membrane 112 and/or within frame 106 to ensure that blood may flow through the one or more of the potential stasis zones 130a, 130b.

In a non-limiting example, FIG. 1E illustrates a stasis reducing feature that includes a plurality of grooves 140 positioned along an internal surface of the frame 106 while the membrane 112 is shown positioned along one or more walls of frame 106. To prevent blood stasis, the grooves 140 may function as conduits (e.g., gutters, paths, orifices, etc.) to move blood along an inner surface of the membrane 112 or to move blood along an outer surface of the frame 106. In the example in which the grooves 140 are part of the frame 106, the grooves 140 can allow a portion of blood to flow within along at least a portion of the surface of the frame and/or surface of the membrane. In the example in which the grooves 140 are part of the membrane 112, the grooves 140 can allow blood flow to occur in the grooves along the internal walls of the membrane 112 to reduce eddies and blood pooling at or adjacent to the outflow end 104.

As shown in FIG. 1E, the grooves 140 are part of the frame 106 and are depicted substantially parallel to longitudinal axis L, however one skilled in the art would appreciate that any shaped path that allows blood to flow from the inflow end 102 to the outflow end 104 may be substituted. One skilled in the art will appreciate that more or fewer grooves may be provided on membrane 112 and such grooves may be angled at any angle from central axis (C) to promote blood flow from a particular stasis zone. The grooves 140 may define a stasis reducing flow path that extends substantially parallel to the central axis (C) and along at least a portion of the inner surface of the frame 106.

In some implementations, grooves 138 and/or grooves 140 may function to ensure laminar flow of blood continues through flow modulating device 100 when device 100 is configured in a substantially open or partially open state. In some implementations, the grooves 138, 140 may include helical shaped paths within the grooves to promote blood to flow through the paths at a particular flow rate. For example, the grooves 138, 140 may be configured with geometry that allows a blood flow rate from the inflow end 102 through to the outflow end 104 of device 100 at a specific speed. The speed may be selected to decrease blood flow or blood flow speed or to increase blood flow or increase blood flow speed through the device 100.

In some implementations, additional stasis reducing features may be included on flow modulating device 100. For example, the device 100 may include one or more perforations (e.g., perforations 142, 144, etc.) adjacent to the outflow end 104 and extending around at least a portion of a surface of the membrane 112. The perforations may be placed near to each stasis zone. For example, a perforation 142 may be positioned on membrane 112 substantially adjacent to stasis zone 130a (FIG. 1C) while a perforation 144 may be positioned on membrane 112 substantially adjacent to stasis zone 130b (FIG. 1C). The perforations 142, 144 may be present with additional perforations (not shown) to increase an amount of blood that may flow from a first surface S1 (FIG. 1A) of membrane 112 to a second surface S2 (FIG. 1) of membrane 112.

In some implementations, the perforations 142, 144 may be combined with other stasis reducing features. For example, in FIG. 1E, the perforations 142, 144 are included on the membrane and may function in combination with the grooves 140 included on the frame 106 to enable blood to flow along portions of the expandable frame 106 from the inflow end 102 to the outflow end 104, which may function to reduce blood stasis around the implantable device 100.

FIG. IF illustrates a side view of the example flow modulating device of FIG. 1A including one or more stasis reducing features. In the embodiment of FIG. 1F, perforations 142, 144 are positioned substantially adjacent to the outflow end 104 of membrane 112 and potential stasis zones 130a, 130b. An additional stasis reducing feature is included, shown here as perforation 146, which may represent an opening to allow for blood flow from the outflow end 104 of the membrane 112 when the membrane is partially or substantially fully closed, occluded, or restricted state. The perforation 146 may ensure that a portion of blood may remain flowing at least partially through the device 100 to ensure that blood stasis does not occur within the membrane 112 while the perforations 142, 144 may ensure that blood stasis does not occur outside the membrane 112 in potential stasis zones 130a, 130b, for example.

FIG. 1G illustrates a perspective view of an example membrane 112 including one or more stasis reducing features for use with the flow modulating devices described herein. As shown, a number of perforations 150, 152, 154, and 156 are formed in the membrane 112 (e.g., along a length of the membrane 112) in a helical arrangement about the central axis (C) and extending at substantially equal intervals from one another along the surface S2 of the membrane.

FIG. 1H illustrates a bottom-up perspective view of the membrane of FIG. 1G. In the depicted example, each of the perforations 150-156 form apertures in the membrane 112 from the outer surface S2 through to the inner surface S1. The perforations 150-156 may allow blood to flow from a plurality of stasis zones (e.g., zones 130a, 130b) to cause a reduction in blood stasis in or around the membrane 112. For example, when blood flows through the perforations 150-156, potential stasis zones may be eliminated or substantially reduced because the perforations 150-156 are aligned in a helical layout. The helical layout provides for a helical flow path, which induces blood to flow in a helical pattern. The helical pattern (shown by arrow 158) of blood flow may provide an advantage of preventing thrombus in or around the potential stasis zones because the helical flow path encourages flushing at a particular flow velocity for deterring blood pooling and/or deterring stasis zones.

FIG. 1I illustrates a bottom-up perspective view of an example membrane including one or more stasis reducing features for use with the flow modulating devices described herein. The example membrane may be membrane 112, shown here in a partially occluded or restricted state, which may at least partially allow blood to flow along the longitudinal axis (L) through a lumen 160 of the membrane 112 and from the inflow end 102 to the outflow end 104.

The stasis reducing features in the embodiment of FIG. 1I include perforations 162, 164, and 166 positioned substantially adjacent to the outflow end 104 of the membrane 112. For example, perforation 162 is positioned within a stasis zone 168 to allow blood to flow from the stasis zone 168 to reduce or eliminate blood stasis in or around the membrane 112. Similarly, perforation 164 is positioned within a stasis zone 170 to allow blood to flow from the stasis zone 170 to reduce or eliminate blood stasis in or around the membrane 112. The perforation 166 is positioned within a stasis zone 172 to allow blood to flow from the stasis zone 172 to reduce or eliminate blood stasis in or around the membrane 112.

The perforations 162, 164, 166 are apertures in the membrane 112. In this example, the perforations 162, 164, and 166 are formed as arc-shaped flaps in the membrane 112. The arch-shaped flaps may open to allow blood to flow through the lumen 160 when blood impinges on a surface of the membrane 112 on or near the particular flap (e.g., perforation 162, 164, and/or 166).

FIG. 1J illustrates a bottom-up perspective view of another example membrane 112 including one or more stasis reducing features for use with the flow modulating devices described herein. The example membrane 112, shown here in a partially occluded or restricted state, which may at least partially allow blood to flow along the longitudinal axis (L) through the lumen 160 of the membrane 112 and from the inflow end 102 to the outflow end 104.

The stasis reducing features in the embodiment of FIG. 1J include perforations 174, 176, and 178 positioned substantially adjacent to the outflow end 104 of the membrane 112. For example, perforation 174 is positioned within the stasis zone 168 to allow blood to flow from the stasis zone 168 to reduce or eliminate blood stasis in or around the membrane 112. Similarly, perforation 176 is positioned within the stasis zone 170 to allow blood to flow from the stasis zone 170 to reduce or eliminate blood stasis in or around the membrane 112. The perforation 178 is positioned within a stasis zone 172 to allow blood to flow from the stasis zone 172 to reduce or eliminate blood stasis in or around the membrane 112.

The perforations 174, 176, and 178 are apertures in the membrane 112. In this example, the perforations 174, 176, and 178 are formed as slots in the membrane 112. The slots may open to allow blood to flow through the lumen 160 when blood impinges on a surface of the membrane 112 on or near the particular flap (e.g., perforation 174, 176, and/or 178). In some implementations, the slots may remain open during operation of device 100, for example, to allow blood to flow through the lumen 160.

As described herein, a perforation may be an aperture in a membrane or frame that is sized and positioned to be effective for allowing blood to pass through the aperture. Example perforation shapes may include, but are not limited to, circles, ovals, squares, rectangles, triangles, polygons, irregular shapes, crescents, semicircles, ellipses, and combinations thereof. In some implementations, the perforations may be formed as slits, slots, flaps, or combinations thereof.

The perforations described herein may be sized to be porous enough to allow blood to flow through the perforation from a first side to a second side of a material in which the perforation is positioned within. For example, the perforations described herein may be sized to be about 0.01 millimeters to about 1 millimeter in depth based on the thickness of the membrane ranging from about 0.01 millimeters to about 1 millimeter in depth. The diameter (e.g., width and/or length) of the perforations described herein may be about 0.5 millimeters to about 2 millimeters based on the devices described herein having a radius of about 5 millimeters to about 30 millimeters.

The perforations described herein may be positioned in any number of layouts along surfaces of the flow modulating devices described herein. Example perforation layouts include, but are not limited to, a single perforation, a substantially helical shaped layout, a clustered layout, a substantially spiral shaped layout, a substantially circular shaped layout, a triangular shaped layout, a substantially straight array or a substantially arced array shaped layout, and combinations thereof.

While three or fewer perforations are depicted in FIGS. 1E, 1F, 1I, and 1J, any number of perforations may be contemplated to function as stasis reducing features in the devices described herein. For example, the number and locations of the perforations described herein may vary to optimize the minimization of stasis and to optimize the function of the flow modulating devices described herein.

Further, any combination of shapes and/or any combination of sizes of the perforations described herein may be contemplated to function as stasis reducing features in the devices described herein. In addition, any of the perforations, grooves, or other stasis reducing features described herein may be combined with any number of other stasis reducing features to reduce stasis within a particular flow modulating device and/or within vessels around or substantially adjacent to the particular flow modulating device.

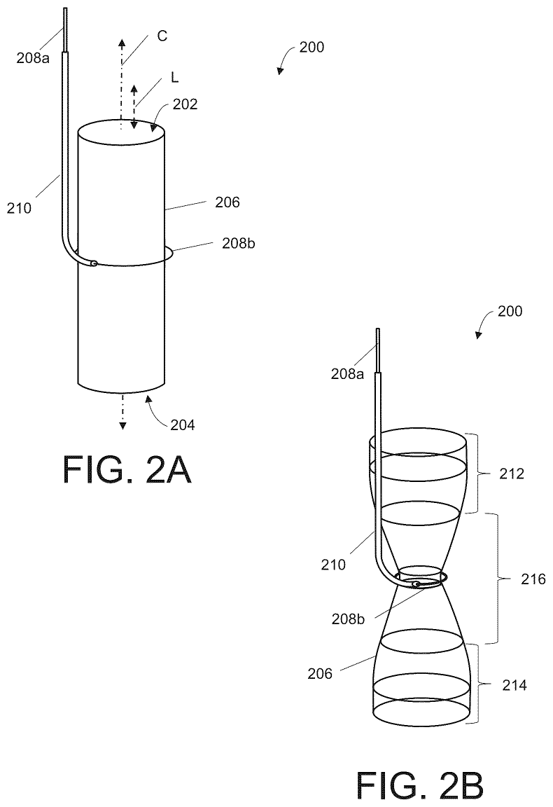

FIGS. 2A-2B illustrate side views of an example flow modulating device 200 for modulating blood flow through a blood vessel. The device 200 may be implanted into a blood vessel, such as the superior vena cava, the inferior vena cava, or any other blood vessel where modulating blood flow is desired. For example, the device 200 may modulate a volume of blood flowing from the superior vena cava into a right atrium to decrease right atrial pressure.

At a high level, the device may include a self-expanding or balloon-expandable frame (e.g., stent) that may be delivered into the blood vessel (e.g., via jugular access, subclavian access, or transfemoral access) using a sheathed catheter (not shown). The frame may include or be coupled to a flexible wire or rope. A flexible catheter tube may form a lasso assembly that loops around a portion of the frame. When tension is applied to the portion of the frame, the frame is circumferentially reduced forming a blood flow restricting orifice. The portion may expand and contract within the blood vessel using a lasso-type control to function as an adjustable blood flow restrictor.

FIG. 2A illustrates a side view of the example implantable device 200 for modulating blood flow through a blood vessel. In this example, the device 200 is shown in an unrestricted blood flow state (e.g., an unconstricted configuration). The unrestricted blood flow state may represent a state of device 200 in which both an inflow 202 and an outflow 204 are open to receive fluid (e.g., blood, drugs, saline, etc.). Fluid flows from the inflow 202 and through a device lumen, about central axis (C), and through to the outflow 204. For example, the device 200 may be in an expanded state when a frame 206 of the device 200 is not occluded or otherwise encumbered from the inflow 202 through to the outflow 204 along longitudinal axis (L) to allow flow of fluid (e.g., blood, drugs, saline, etc.) through the lumen of the device when the device 200 is implanted in a blood vessel.

The device 200 includes an expandable frame (e.g., frame 206) that defines a lumen therethrough (from the inflow 202 to the outflow 204 about longitudinal axis (L)). The frame 206 may be a stent constructed of metal (e.g., stainless steel, platinum, Nitinol® wire or another shape memory alloy) or other material suitable for implantation in the human body. In some examples, the expandable frame 206 is a bare metal stent, such that the expandable frame 206 is configured to be at least partially incorporated into an inner wall of the blood vessel. In some examples, the expandable frame 206 has a pro-endothelialization coating, such that the expandable frame can be at least partially incorporated into an inner wall of the blood vessel. This incorporation may allow a site of the device 200 to maintain a non-thrombogenic, non-immunogenic environment with respect to the device 200. For example, the coating of the frame 206 may be any pro-endothelial factor including, but not limited to, endothelial growth factor, vascular endothelial growth factor, or any related compound.

After the device 200 is implanted, partially implanted, or otherwise incorporated into the blood vessel wall (i.e., tissue may heal around at least a portion of the frame), the frame 206 becomes incorporated into the inner wall of the blood vessel such that cinching a portion of the device 200 has the effect of also cinching the vessel wall/cross-section at (or adjacent to) a cinching location corresponding to the portion of the device 200. Optionally, the frame may be wrapped with a membrane that can form a seal between an outer surface portion (e.g., an outer diameter) of the frame 206 and the inner wall of the blood vessel. The formed seal may prevent leakage in-between the device 200 and the blood vessel inner wall when the cinching occurs.

Referring again to FIG. 2A, the device 200 also includes a control element 208 with a first portion 208a and a second portion 208b connected. In some examples, the first portion 208a and second portion 208b form a single length of material in which the control element 208b may be at least partially circumferentially disposed about the expandable frame 206. The control element portion 208b may extend along or away from an outer diameter of device 200. Pulling or tensioning the first portion 208a may result in cinching the second portion 208b about the device 200. In some examples, the control element is a wire. In some examples, the control element is a suture. In some examples, the control element is a cord or other manually or automatically operated pull wire.

In some examples, the frame 206 may be transitionable between a constricted configuration and an unconstricted configuration. The constricted configuration of the expandable frame 206 enables restriction of blood flow through the lumen of the frame 206. The constricted configuration may enable a partial occlusion or a full occlusion of the blood vessel. The unconstricted configuration may allow blood to flow substantially freely through the lumen of the frame 206.

The device 200 further includes a mechanical actuator 210 for tensioning the control element 208a, 208b to transition the expandable frame 206 to the constricted configuration. For example, the first portion 208a may be pulled to cinch the second portion 208b. The mechanical actuator 210 may also trigger release of tension in the control element 208a, 208b by releasing the first portion 208a, which can transition the expandable frame 206 to the unconstricted configuration.

FIG. 2B illustrates a side view of the example flow modulating device 200 in a restricted blood flow state. The device 200 includes the frame 206, control element 208a, 208b, and the actuator 210. In this example, the device 200 includes the frame 206, depicted for clarity of discussion, with a first end portion 212, a second end portion 214, and an intermediate portion 216. In some examples, the frame 206 is seamless without actual breaks in the outer surface. In some examples, the frame 206 is assembled in separate componentry that combine to form the first end portion 212, the intermediate portion 216, and the second end portion 214 of the frame 206. Here, the control element portion 208b is at least partially circumferentially disposed about the intermediate portion 216 of the expandable frame 206 and the intermediate portion 216 is cinched, thereby placing the device 200 in the constricted configuration. The cinching may be triggered by the actuator 210 to tension the control element 208a, 208b to transition the intermediate portion 216 of the expandable frame 206 to the constricted configuration. An uncinching can be triggered by the actuator 210 to release tension in the control element 208a, 208b to transition the intermediate portion 216 of the expandable frame to the unconstricted configuration, as shown in FIG. 2A.

In some examples, the actuator 210 is a catheter. In such examples, the control element 208a, 208b may be axially translatable through a lumen of the catheter to tension or release tension in the control element 208a, 208b. For example, pulling or pushing the control clement 208a (e.g., a wire or suture) through the lumen of the catheter (e.g., functioning as the actuator 210) may tension or release tension in the control element portion 208b to put the device 200 into a restricted blood flow state or an unrestricted blood flow state, respectively.

In some examples, the actuator 210 is a linear actuator. The linear actuator may be, for example, a pneumatic linear actuator, an electromechanical linear actuator, or a hydraulic linear actuator. The linear actuator may be used to tension or release tension in the control clement portion 208a, 208b to put the device 200 into a restricted blood flow state or an unrestricted blood flow state, respectively.

In some examples, the actuator 210 includes a pump (not shown) fluidly connected to a reservoir (not shown); a chamber (not shown) having a first portion and a second portion; a manifold (not shown) fluidly connected to the pump, the reservoir, and the chamber; and a piston (not shown) coupled to the control element portions 208a or 208b. In such examples, movement of the piston between the first portion and the second portion of the chamber releases tension on and applies tension to the control element portions 208a or 208b, respectively.

For example, the first portion of the chamber may be ported in such a way that the piston manipulates the control element portion 208a to place device 200 into a restricted blood flow state when moved by fluid flowed into the first portion of the chamber or to an unrestricted blood flow state when fluid is evacuated from the first portion of the chamber. The actuation fluid of the actuator may be a gas or a liquid, i.e., the actuator 210 may be hydraulically or pneumatically actuated. The actuation fluid may also be rechargeable either by pressurizing a gas or by creating stored mechanical energy, i.e., pumping liquid into an elastic bladder or into a pressure tank. The actuator 210 can include a power source responsible for powering the pump or any other power-able devices. This stored power source may be electrical and used to power an electromechanical pump. Other embodiments may include a pump powered by an anatomy of the user.

In some examples, the device 200 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator 210, the microprocessor 1008, and the sensor 1006. For example, the sensor 1006 may sense characteristics of blood flow in the blood vessel (e.g., blood pressure) and may cause the microprocessor to provide signals to the actuator 210 (e.g., actuation device 1012) and/or control element portions 208a, 208b (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to tension the control clement portions 208a, 208b, or release tension in the control element portions 208a, 208b based on the sensed pressure in the blood vessel.

In some examples, the sensor 1006 may be communicatively coupled to device 200. The sensor 1006 may include one or more of an image sensor, a strain gauge, a piezoelectric sensor, a capacitance sensor, and/or a vacuum pressure sensor. When the device 200 is coupled to a power source (e.g., power source 1014), the power source may include an induction coil in the event that the power source includes magnets and/or coils. The induction coil may be used to operate one or more of such magnets, as described in further detail in FIGS. 11A-11B.

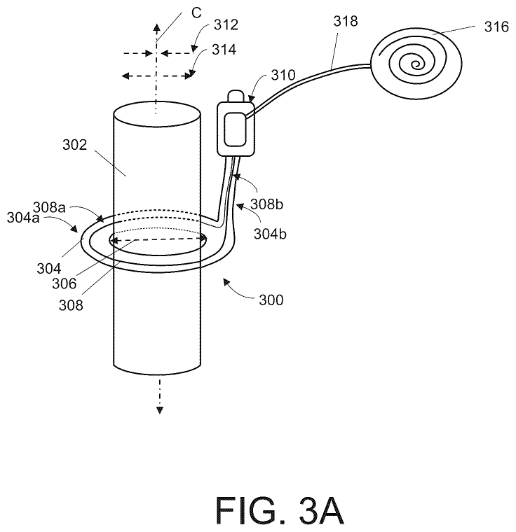

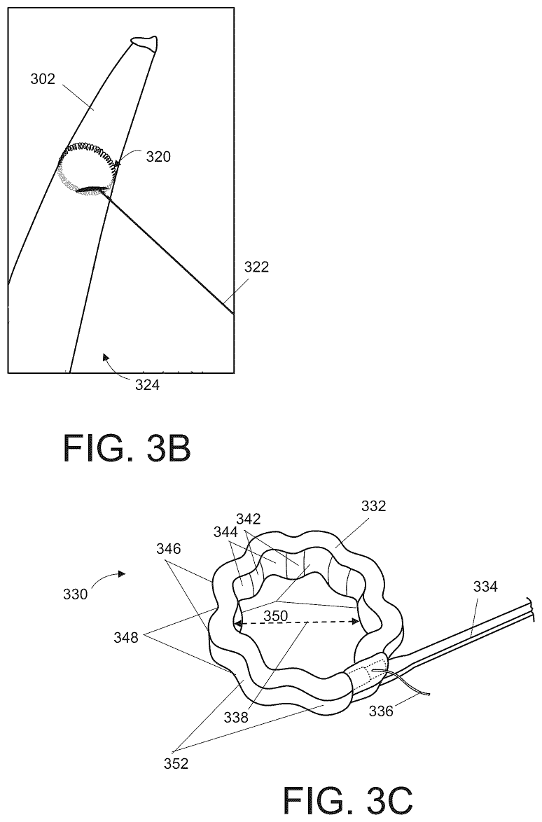

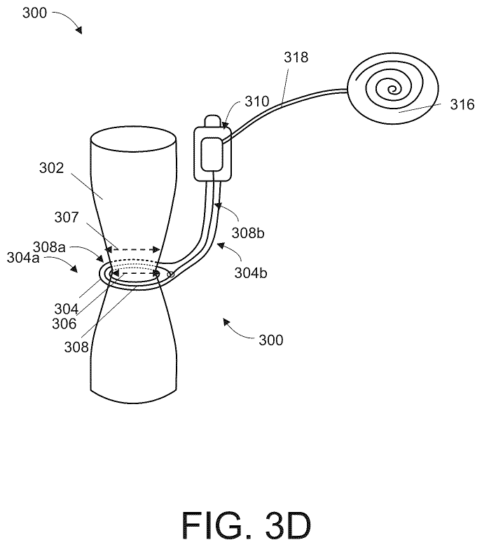

FIGS. 3A-3D illustrate perspective views of an example flow modulating device 300 for surrounding a portion of a blood vessel to manage flow through the blood vessel. The device 300 may be implanted into a blood vessel, such as the superior vena cava, the inferior vena cava, or any other blood vessel where modulating blood flow is desired. For example, the device 300 may modulate a volume of blood flowing from the superior vena cava into a right atrium to decrease right atrial pressure.

The device 300 can surround an outside portion of the blood vessel to restrict blood flow, rather than being implanted within the blood vessel, as described in FIGS. 1A-2B. Briefly, the device 300 may include a lasso-shaped assembly that may be placed on an outside diameter of a blood vessel. The lasso-shaped assembly may be tensioned or released from tension to place the device 300 into a restricted blood flow state or an unrestricted blood flow state, respectively. A polymer or spring element may be integrated into the lasso-shaped assembly to allow for expansion when tension is removed from the lasso-shaped assembly. Sensors may be used to determine pressure changes in the vessel.

FIG. 3A illustrates a side view of an example flow modulating device 300 in an unrestricted blood flow state. The unrestricted blood flow state may represent a state of device 300 in which fluid (e.g., blood, drugs, saline, etc.) may flow freely through the blood vessel 302. For example, the device 300 may be in an expanded state when the device 300 is not constricting (e.g., squeezing) the outer wall of the blood vessel 302.

The device 300 includes a substantially annular shaped ring 304 with a first end 304a and a second free end 304b. The device 300 can be at least partially circumferentially disposed about an outer wall portion of the blood vessel 302, as shown adjacent to inner diameter 306 of the ring 304. The device 300 also includes a control element 308 that is, at least partially, circumferentially disposed in the ring 304. The control element 308 has a first looped end 308a and a second free end 308b that passes through the first looped end 308a. The control element 308 may function to translate relative to the inner diameter 306 of the ring 304.

The device 300 also includes an actuator 310 (e.g., a mechanical actuator) that is coupled to the second free end 304b of the control element 308 to actuate the device 300 into an unrestricted blood flow state or a restricted blood flow state or any state therebetween. For example, the actuator 310 may cause the device 300 to tension the control element 308 to reduce the inner diameter 306 about a central axis (C), as shown by directional arrows 312. The actuator 310 may further cause the device 300 to release the tension in the control element 308 to maintain or increase the inner diameter 306 about the central axis (C), as shown by directional arrows 314.

In some examples, the actuator 310 is a motor connected to power source 316 (e.g., an inductive coil) via a catheter/wire 318. The power source 316 may be activated (e.g., magnetically induced in the example of an inductive coil) to cause the motor to tension or release tension from the control element 308.

In some examples, the actuator 310 is a catheter (e.g., catheter/wire 318). In such examples, the control element 308 may be axially translatable through a lumen of the catheter to tension or release tension in the control element 308. For example, pulling or pushing the control element 308 (e.g., represented as a wire or a suture) through the lumen of the catheter (e.g., functioning as the actuator 310) may tension or release tension in the control element 308 to put the device 300 into a restricted blood flow state or an unrestricted blood flow state, respectively.

In some examples, the actuator 310 is a linear actuator. The linear actuator may be, for example, a pneumatic linear actuator, an electromechanical linear actuator, or a hydraulic linear actuator. The linear actuator may be used to tension or release tension in the control element 308 to put the device 300 into a restricted blood flow state or an unrestricted blood flow state, respectively.

In some examples, the actuator 310 includes a pump (not shown) fluidly connected to a reservoir (not shown); a chamber (not shown) having a first portion and a second portion; a manifold (not shown) fluidly connected to the pump, the reservoir, and the chamber; and a piston (not shown) coupled to the control element 308. In such examples, movement of the piston between the first portion and the second portion of the chamber releases tension on and applies tension to the control element 308, respectively.

For example, the first portion of the chamber may be ported in such a way that the piston manipulates the control element 308 to place device 300 into a restricted blood flow state when moved by fluid flowed into the first portion of the chamber or to an unrestricted blood flow state when fluid is evacuated from the first portion of the chamber. The actuation fluid of the actuator may be a gas or a liquid, i.e., the actuator 310 may be hydraulically or pneumatically actuated. The actuation fluid may also be rechargeable either by pressurizing a gas or by creating stored mechanical energy, i.e., pumping liquid into an elastic bladder or into a pressure tank. The actuator 310 can include a power source responsible for powering the pump or any other power-able devices. This stored power source may be electrical and used to power an electromechanical pump. Other embodiments may include a pump powered by an anatomy of the user.

In some examples, the device 200 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator 210, the microprocessor 1008, and the sensor 1006. For example, the sensor 1006 may sense characteristics of blood flow in the blood vessel (e.g., blood pressure) and may cause the microprocessor to provide signals to the actuator 210 (e.g., actuation device 1012) and/or control element portions 208a, 208b (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to tension the control clement portions 208a, 208b, or release tension in the control element portions 208a, 208b based on the sensed pressure in the blood vessel.

In some examples, the sensor 1006 may be communicatively coupled to device 200. The sensor 1006 may include one or more of an image sensor, a strain gauge, a piezoelectric sensor, a capacitance sensor, and/or a vacuum pressure sensor. When the device 200 is coupled to a power source (e.g., power source 1014), the power source may include an induction coil in the event that the power source includes magnets and/or coils. For example, the actuator 310 may be a motor and the first looped end 308a may be attached to a lead screw coupled to the motor and further operatively coupled to a nut. The nut may be operatively coupled to the control element 308. When a magnet (see FIGS. 11A-11B.) is driven to rotate and excite the inductive coil (e.g., and power the actuator/motor), the nut can move along the lead screw to cause the actuator 310 to tension or release tension from the control element 308.

FIG. 3B illustrates a substantially circular spring 320 substantially circumferentially disposed about the blood vessel 302. In some examples, the spring 320 may replace the ring 304. The spring 320 is operably coupled to a control wire 322. The control wire 322 may tension or release tension in the spring 320 to put the device 300 into a restricted blood flow state or an unrestricted blood flow state, respectively. For example, the user 324 may actuate the control wire 322 to tension the control wire 322 and thus the spring 320 to reduce the inner diameter of the vessel 302 or release the tension in the control wire 322/spring 320 to maintain or increase the inner diameter of the vessel 302 about the central axis (C). In some examples, the spring 320 may provide an advantage of allowing the blood vessel to naturally expand to accommodate blood flow surge upon relaxation of the spring 320, for example, when tension is removed from the control wire 322.

FIG. 3C illustrates an example blood flow modulating device 330. The device 330 includes a ring 332, an actuator 334, and a control wire 336. The ring 332 may be substantially circumferentially disposed about a blood vessel (not shown). In some examples, the ring 332 is a polymeric ring formed from one or more of: silicone, poly (urethane), poly (acrylate), or poly (ethylene vinyl acetate). In some examples, the ring 332 is a textile as described elsewhere herein. In some examples, the ring 332 is formed of tissue, as described elsewhere herein.

The ring 332 includes a central cavity in which a control wire 336 is threaded therethrough. The control wire 336 is operably coupled to the actuator 334. The actuator 334 may activate the control wire 336 to tension or release tension in the ring 332 to put the device 330 into a restricted blood flow state or an unrestricted blood flow state, respectively. For example, the actuator 334 may actuate the control wire 336 to tension the ring 332 to reduce a ring diameter 338, and thus reduce the diameter of the vessel.

An inner surface 350 of the ring 332 may be formed with an irregular surface where the surface is a substantially serpentine surface that forms a closed ring. For example, the inner perimeter/surface 350 of ring 332 includes a plurality of serpentine bends having inner wall protrusions 342 connected to inner wall indentations 344 in a pattern to form a shallow serpentine inner ring. Although a serpentine surface is described, any surface configuration or conformation is contemplated herein including a substantially smooth inner perimeter, a beveled inner perimeter, a rough inner perimeter that is a friction fit on an outer surface of the blood vessel, and the like.

Similarly, the actuator 334 may actuate the control wire 336 to release tension in the ring 332 to increase or maintain the ring diameter 338, and thus increase or maintain the diameter of the blood vessel. An outer surface 352 of the ring 332 may be formed with an irregular surface around the perimeter of the ring 332 where the surface is a substantially serpentine surface that forms a closed ring. For example, the outer perimeter of ring 332 includes a plurality of serpentine bends having inner wall indentations 344) having outer wall protrusions (e.g., outer wall protrusions 346 connected to outer wall indentations 348 in a pattern to form a serpentine outer ring. Although a serpentine surface is described, any surface configuration or conformation is contemplated herein including a substantially smooth outer perimeter, a beveled outer perimeter, and the like.

In some examples, the ring 332 may provide an advantage of allowing the blood vessel to naturally expand to accommodate blood flow surge upon relaxation of the polymeric ring 332 and protrusions 342, 346, for example, when tension is removed from the control wire 336.

FIG. 3D illustrates a side view of the example flow modulating device 300 in a restricted blood flow state. To place the device 300 in the restricted blood flow state, the actuator 310 may trigger the application of tension to the second free end 304b to reduce the inner diameter 306 of the control clement 308 disposed in the ring 304. The actuator 310 may further trigger the reduction of tension on the second free end 304b to maintain or increase the diameter 306 of the control element 308 disposed in the ring 304. As shown in this example, applying tension to the second free end 304b resulted in reducing a diameter 307 of the blood vessel 302. In some examples, the reduction of the tension on the second free end 304b may result in maintaining or increasing the diameter of the blood vessel 302.

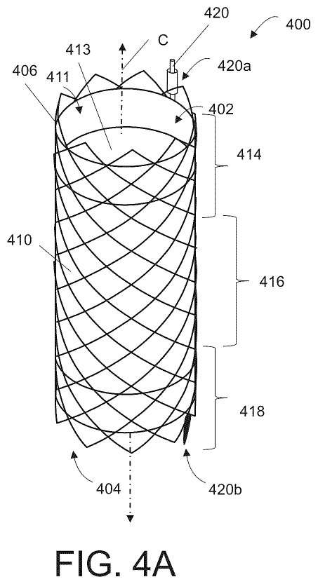

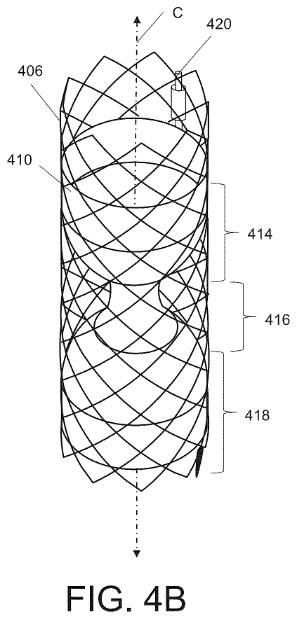

FIGS. 4A-4B illustrate perspective views of an example flow modulating device 400 for modulating blood flow through a blood vessel. The device 400 may be implanted into a blood vessel, such as the superior vena cava, the inferior vena cava, or any other blood vessel where modulating blood flow is desired. For example, the device 400 may modulate a volume of blood flowing from the superior vena cava into a right atrium to decrease right atrial pressure.

At a high level, the device may include an assembly including a self-expanding or balloon-expandable frame (e.g., stent) and a membrane installed within the lumen of the frame. The assembly may be delivered into the blood vessel (e.g., via jugular access, subclavian access, or transfemoral access) using a sheathed catheter (not shown). The frame may include or be coupled to a flexible wire, suture, or rope. When tension is applied to the flexible wire, suture, or rope, or a portion of the frame, the frame and/or membrane is circumferentially reduced forming a blood flow restricting orifice. The portion of the membrane may expand and contract within the blood vessel to function as an adjustable blood flow restrictor.

FIG. 4A illustrates a top-down perspective view of an example flow modulating device 400 in an unrestricted blood flow state. The device 400 includes an expandable frame (e.g., frame 406) that defines a lumen therethrough (from the inflow 402 to the outflow 404 about a central axis (C)). The frame 406 may be a stent comprising or constructed of metal (e.g., stainless steel, platinum, Nitinol® wire or another shape memory alloy), or other material suitable for implantation in the human body. In some examples, the expandable frame 406 is a bare metal stent, such that the expandable frame 406 is configured to be at least partially incorporated into an inner wall of the blood vessel. In some examples, the expandable frame 406 has a pro-endothelialization coating, such that the expandable frame can be at least partially incorporated into an inner wall of the blood vessel. This incorporation may allow a site of the device 400 to maintain a non-thrombogenic, non-immunogenic environment with respect to the device 400. For example, the coating of the frame 406 may be any pro-endothelial factor including, but not limited to, endothelial growth factor, vascular endothelial growth factor, or any related compound.

After the device 400 is implanted, partially implanted, or otherwise incorporated into the blood vessel wall (i.e., tissue may heal around at least a portion of the frame), the frame 406 becomes incorporated into the inner wall of the blood vessel. Optionally, the frame may be wrapped with a membrane that can form a seal between an outer surface portion (e.g., an outer diameter) of the frame 406 and the inner wall of the blood vessel.

The device 400 may include a substantially tubular-shaped membrane 410 provided within a partial length of the lumen 411 of the frame 406. In some examples, the membrane 410 may be a stent that is installed within the frame 406 (e.g., another stent). The membrane 410 may be formed of a polymer, a copolymer, a shape memory alloy, a textile (e.g., woven, knitted, nonwoven, or braided), a tissue (e.g., bovine pericardium, equine pericardium, porcine vena cava, etc.), or a combination thereof. The membrane 410 may be actuated by one or more control elements to cause the membrane 410 to reshape. The reshaping can cause a narrowing and/or closure in a portion of the membrane 410, which may cause a narrowing and/or closure of the blood vessel in which device 400 is installed. In some examples, the membrane 410 may be a single member. In some examples, the membrane 410 may be multiple members composed of one or more materials that are coupled together; removably attached together; or fixedly attached together.

As shown in FIG. 4A, the membrane 410 includes a first end portion 414, an intermediate portion 416, and a second end portion 418. The device 400 also includes a control element 420 disposed within a portion of the frame. The control element 420 may be a wire, a cord, a suture, or the like. The control element 420 includes a proximal end 420a and a distal end 420b. The proximal end 420a may be coupled to the membrane 410. The distal end 420b may be coupled to a mechanical actuator (e.g., actuation device 1108 of FIGS. 11A-11B or actuation devices 1012 of FIG. 10). The mechanical actuator may be configured to actuate the control element 420 to cause the frame 406 to transition between the constricted configuration and the unconstricted configuration.

In some examples, the actuator coupled to control element 420 is a catheter. In such examples, the control element 420 may be axially translatable through a lumen of the catheter to tension or release tension in the control element 420. For example, pulling or pushing the control element 420 (e.g., a wire or suture) through the lumen of the catheter (e.g., functioning as the actuator) may tension or release tension in the control element 420 to put the device 400 into a restricted blood flow state or an unrestricted blood flow state, respectively.

In some examples, the actuator coupled to control element 420 is a linear actuator. The linear actuator may be, for example, a pneumatic linear actuator, an electromechanical linear actuator, or a hydraulic linear actuator. The linear actuator may be used to tension or release tension in the control element 420 to put the device 400 into a restricted blood flow state or an unrestricted blood flow state, respectively.

In some examples, the device 400 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator, the microprocessor 1008, and the sensor 1006. For example, the sensor 1006 may sense characteristics of blood flow in the blood vessel (e.g., blood pressure) and may cause the microprocessor to provide signals to the actuator (e.g., actuation device 1012) and/or control clement 420 (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to tension the control clement 420, or release tension in the control element 420 based on the sensed pressure in the blood vessel.

In some examples, the sensor 1006 may be communicatively coupled to device 400. The sensor 1006 may include one or more of an image sensor, a strain gauge, a piezoelectric sensor, a capacitance sensor, and/or a vacuum pressure sensor. If the device 400 is coupled to a power source (e.g., power source 1014), the power source may include an induction coil in the event that the power source includes magnets and/or coils. The induction coil may be used to operate one or more of such magnets, as described in further detail in FIGS. 11A-11B.

FIG. 4B illustrates a top-down perspective view of the example flow modulating device 400 in a restricted blood flow state. The restricted blood flow state for device 400 represents a constricted configuration of the membrane 410. In particular, the intermediate portion 416 of the membrane 410 can be radially narrowed when the device 400 is placed in the restricted blood flow state while the first end portion 414 and the second end portion 418 of the membrane 410 are translated toward the intermediate portion 416. In some examples, the intermediate portion 416 of the membrane 410 can be pinched such that two points along the circumference of the intermediate portion of membrane 410 are pulled toward the central axis (C) while the other points along the circumference move to form an oval or ellipse shape within an inner wall 413 of the membrane 410. When the intermediate portion 416 is narrowed, a length of one or both of the end portions 414, 418 may shift longitudinally (e.g., towards an intermediate portion 416) within the frame 406 to accommodate the narrowing of the intermediate portion 416.

As shown in FIG. 4B, the intermediate portion 416 has narrowed radially toward the central axis (C) while the two end portions 414, 418 begin to taper at junctions near to (e.g., adjacent to) the intermediate portion 416, as compared to the intermediate portion 416 of FIG. 4A. In some examples, the end portions 414, 418 may instead translate along the central axis (C) without a shape change allowing the intermediate portion 416 to change shape while maintaining a shape of both end portion 414 and end portion 418.

In some examples, the narrowing of the intermediate portion 416 of the membrane 410 may result in forming a dumbbell (e.g., or dog bone) shape within the inner wall of the membrane 410 at the intermediate portion 416. The dumbbell shape can reduce an amount of blood allowed to flow through the blood vessel.

When device 400 is in the constricted configuration (e.g., the constricted blood flow state), the frame 406 and/or the membrane 410 may be caused to change shape to restrict blood flow through the membrane 410. For example, one or more struts of the frame 406 may function to be tensioned or released from tension to cause the membrane 410 to tension or release from tension. In some examples, the membrane 410 is narrowed at the intermediate portion 416 and the frame 406 follows the same (or similar) motion and/or shape change as the membrane 410, such that the frame 406 is at least partially adhered to the membrane 410.

In some examples, placing the device 400 in the constricted configuration causes a reduction in the blood flow through the membrane 410 by at least about 50 percent by reducing a cross-sectional area of the inner portion 413 of the membrane 410. In some examples, placing the device 400 in the constricted configuration causes a reduction in the blood flow through the membrane 410 by at least about 50 percent to about 75 percent by reducing a cross-sectional area of an inner portion of the membrane 410.

When the device 400 is placed into the unconstricted configuration (e.g., the unconstricted blood flow state), the frame 406 and/or the membrane 410 may expand at or near to the intermediate portion 416 of the membrane 410 while the first end portion 414 and the second end portion 418 of the membrane 410 are translated away from the intermediate portion. An example of the expanded membrane is shown in FIG. 4A.

In some examples, the device 400 may be mechanically actuated using a mechanical actuator (e.g., actuation device 1108 of FIGS. 11A-11B or actuation devices 1012 of FIG. 10). The mechanical actuator can cause the intermediate portion 416 to transition to the constricted configuration. The constricted configuration may include constriction of the intermediate portion 416, which may cause regions adjacent to the constricted region to expand radially and/or linearly while the first end portion 414 and the second end portion 418 translate longitudinally away from the intermediate portion 416. Alternatively, or additionally, the first end portion 414 and second end portion 418 may increase in length. For example, the increase in length may be based on material being stretched or released from tension. The increase in length can occur while the end portions 414, 418 are translated away from the intermediate portion 416 and while the first end portion 414 remains coupled to the intermediate portion 416 and the intermediate portion 416 remains coupled to the second end portion 418.

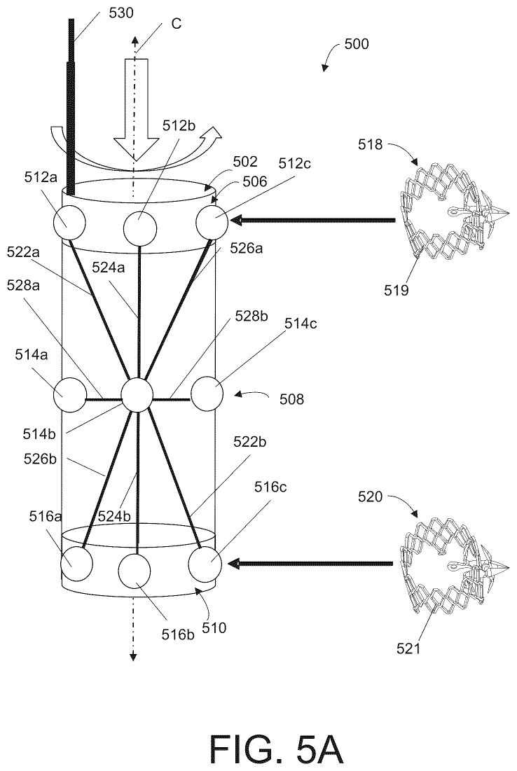

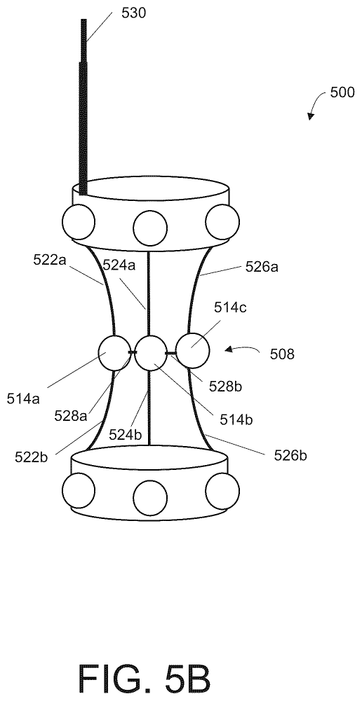

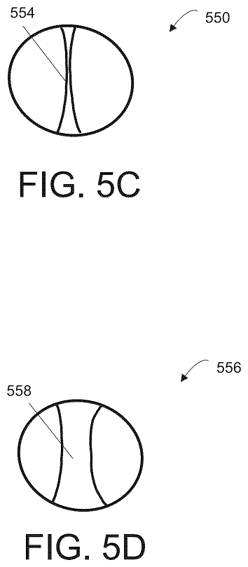

FIGS. 5A-5B illustrate perspective views of an example flow modulating device 500 for modulating blood flow through a blood vessel. In this example, the device 500 is shown in an unrestricted blood flow state (e.g., an unconstricted configuration). The unrestricted blood flow state may represent a state of device 500 in which an inflow 502 and an outflow 504 are both open to receive fluid (e.g., blood, drugs, saline, etc.) from the inflow 502 and through a device lumen, defined about central axis (C), and through to the outflow 504.

FIG. 5A illustrates a side perspective view of the example flow modulating device 500 in an unrestricted blood flow state. The device 500 includes a frame structure that includes at least a first annular member 506, a second annular member 508, a third annular member 510, and a plurality of anchors, and a plurality of frame wires. The first annular member 506 may include two or more first end anchors (e.g., first end anchor 512a, first end anchor 512b, first end anchor 512c, etc.). The two or more first end anchors (e.g., first end anchor 512a, first end anchor 512b, first end anchor 512c, etc.) may be arranged circumferentially around the first annular member 506.

The second annular member 508 may include two or more intermediate anchors (e.g., intermediate anchor 514a, intermediate anchor 514b, intermediate anchor 514c, etc.). The two or more intermediate anchors (e.g., intermediate anchor 514a, intermediate anchor 514b, intermediate anchor 514c, etc.) may be arranged circumferentially around the second annular member 508.

The third annular member 510 may include two or more second end anchors (e.g., second end anchor 516a, second end anchor 516b, second end anchor 516c, etc.). The two or more second end anchors (e.g., second end anchor 516a, second end anchor 516b, second end anchor 516c, etc.) may be arranged circumferentially around the third annular member 510.

The annular members 506-510 and/or the plurality of anchors (e.g., anchors 512a-512c, 514a-514c, 516a-516c, etc.) may be constructed of metal (e.g., stainless steel, platinum, Nitinol® wire, or another shape memory alloy or polymer) or other material suitable for implantation in the human body.

The device 500 includes a plurality of frame wires including at least frame wires 522a, 522b, 524a, 524b, 526a, 526b, 528a, and 528b. The frame wires 522a, 524a, and 526a can connect each of the two or more first end anchors (e.g., end anchors 512a, 512b, 512c, etc.) to a respective intermediate anchor (e.g., intermediate anchors 514a, 514b, 514c, etc.). The plurality of frame wires including at least frame wires 522b, 524b, and 526b can connect each of the two or more intermediate anchors (e.g., anchors 514a, 514b, 514c, etc.) to a respective end anchor (e.g., 516a, 516b, 516c) such that each intermediate anchor 514a, 514b, 514c, etc. is connected to a single anchor in annular member 506 and a single anchor in annular member 510. In addition, the intermediate anchors 514a, 514b, 514c, etc. may be connected as well. For example, intermediate anchor 514a may be connected to intermediate anchor 514b by frame wire 528a and intermediate anchor 514b may be connected to intermediate anchor 514c by frame wire 528b.

In some examples, the frame wire section 522a and 522b may be a single frame wire for connecting anchors between annular members 506-510. In some examples, the frame wire section 522a and 522b may be formed by two or more frame wires for connecting anchors between annular members 506-510.

In some examples, each of the two or more first end anchors (e.g., end anchor 512a, 512b, 512c, etc.) may include a first stent portion (e.g., stent portion 518) with an outer diameter 519 configured to substantially form a seal with an inner wall of the blood vessel in which the device 500 is implanted. In such an example, each of the two or more intermediate anchors (e.g., intermediate anchor 514a, 514b, 514c, etc.) may include or be coupled to a spring element (not shown) with a spring portion (not shown) having an outer diameter that may substantially form a seal with the inner wall of the blood vessel in which device 500 is implanted. In the same example, the two or more second end anchors (e.g., end anchor 516a, 516b, 516c, etc.) may include a second stent portion (e.g., stent portion 520) with an outer diameter 521 configured to substantially form a seal with the inner wall of the blood vessel in which the device 500 is implanted.

In some examples, each of the two or more first end anchors (e.g., end anchor 512a, 512b, 512c, etc.) may include a first stent portion (e.g., stent portion 518) with an outer diameter configured to substantially form a seal with an inner wall of the blood vessel. In such an example, each of the two or more intermediate anchors (e.g., anchors 514a, 514b, 514c, etc.) may include a second stent portion (e.g., stent portion 520) with an outer diameter configured to substantially form a seal with the inner wall of the blood vessel in which the device 500 is implanted. In the same example, the two or more second end anchors (e.g., end anchor 516a, 516b, 516c, etc.) may include a third stent portion (similar to stent portions 518, 520) with an outer diameter configured to substantially form a seal with the inner wall of the blood vessel in which the device 500 is implanted.

In some examples, the frame wire section 522a and 522b may be a single frame wire for connecting anchors between annular members 506-510. In some examples, the frame wire section 522a and 522b may be two or more frame wires for connecting anchors between annular members 506-510. In some examples, the frame wire section 524a and 524b may be a single frame wire for connecting anchors between annular members 506-510. In some examples, the frame wire section 524a and 524b may be two or more frame wires for connecting anchors between annular members 506-510. In some examples, the frame wire section 526a and 526b may be a single frame wire for connecting anchors between annular members 506-510. In some examples, the frame wire section 526a and 526b may be two or more frame wires for connecting anchors between annular members 506-510.

In some examples, the second annular member 508 is a wire member comprising or formed of a shape memory polymer. The wire member may connect the two or more intermediate anchors (e.g., anchor 514a, anchor 514b, anchor 514c, etc.) in sequence around the annular member 508. In some examples, the mechanical actuator 130 may tension the wire member (representing annular member 508) to transition the second annular member 508 to the constricted configuration (FIG. 5B) and to release tension in the wire member (representing annular member 508) to transition the second annular member 508 to the unconstricted configuration (FIG. 5A).

The plurality of frame wires 522a-528b may comprise or be constructed of metal (e.g., stainless steel, platinum, Nitinol® wire, or another shape memory alloy or polymer) or other material suitable for implantation in the human body. The frame wires 522a-528b may be tensioned or released from tension to constrict (e.g., narrow) an aperture through the second annular member 508 to restrict blood flow through the blood vessel in which the device 500 is installed by constricting the frame members and partially occluding the blood vessel.

The device 500 may also include a mechanical actuator 530 to tension the plurality of frame wires (e.g., any one or more frame wires 522a-528b) to transition the second annular member 508 to the constricted configuration and to release tension in the plurality of frame wires (e.g., any one or more frame wires 522a-528b) to transition the second annular member 508 to the unconstricted configuration.

In operation, the second annular member 508 may be transitionable between a constricted configuration and an unconstricted configuration in response to receiving an input from the mechanical actuator 530. The unconstricted configuration is shown in FIG. 5A with the device 500 expanded at the intermediate/second annular member 508 while the constricted configuration is shown in FIG. 5B, fully occluded in FIG. 5C, and partially occluded in FIG. 5D.

In some examples, the plurality of frame wires (e.g., any of frame wires 522a-528b) may comprise or be composed of a shape memory alloy to allow two or more intermediate anchors to bend radially toward the central (C) axis of the device 500 when the plurality of frame wires is tensioned in response to receiving an actuation signal from actuator 530, for example. Releasing tension from one or more of the plurality of frame wires 522a-528b may result in configuring the second annular member 508 in an unrestricted blood flow state (e.g., FIG. 5A), as described elsewhere herein.

In some examples, the plurality of frame wires (e.g., any of frame wires 522a-528b) may comprise or be composed of a shape memory alloy to allow two or more intermediate anchors to and to move radially away from the central axis (C) of the device 500 when the plurality of frame wires are released from tension, in response to receiving an actuation signal from actuator 530, for example.

FIG. 5B illustrates a side perspective view of the example flow modulating device 500 in a restricted blood flow state. Providing tension in one or more of the plurality of wires 522a-528b may result in configuring the second annular member 508 (and device 500) in a restricted blood flow state. For example, two or more intermediate anchors 514a, 514b, 514c, etc. can attach to an inner wall of the blood vessel in which the device 500 is implanted. In such an example, any one or more of frame wires 522a-528b may comprise or be composed of a shape memory alloy and may be placed under tension to partially collapse the blood vessel at the second annular member 508 or release tension to expand the blood vessel at the second annular member 508 (FIG. 5A).

In some examples, the device 500 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator 530, the microprocessor 1008, and the sensor 1006. For example, the sensor 1006 may sense characteristics of blood flow in the blood vessel (e.g., blood pressure) and may cause the microprocessor to provide signals to the actuator 530 (e.g., actuation device 1012) and/or control element (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to the mechanical actuator 530 to either tension one or more frame wires 522a-528b or release tension in one or more frame wires 522a-528b based on the sensed pressure in the blood vessel.

In some examples, the sensor 1006 may be communicatively coupled to device 500. The sensor 1006 may include one or more of an image sensor, a strain gauge, a piezoelectric sensor, a capacitance sensor, and/or a vacuum pressure sensor. If the device 500 is coupled to a power source (e.g., power source 1014), the power source may include an induction coil in the event that the power source includes magnets and/or coils. The induction coil may be used to operate one or more of such magnets, as described in further detail in FIGS. 11A-11B.

Although three anchors (e.g., 512a-512c, 514a-514c, 516a-516c are shown in FIGS. 5A-5B, any number of anchors may be provided around the circumference of annular members 506-508. For example, each annular member 506-508 may include two anchors, four anchors, five anchors, six anchors, seven anchors, eight anchors, etc. The plurality of frame wires may be increased or decreased to connect a selected number of anchors on annular member 506 to anchors on annular member 507 and further connect such anchors from annular member 507 to anchors on annular member 508.

In some examples, the anchors 512a-512c; the anchors 514a-514b; and/or the anchors 516a-516c may be replaced with frame portions (e.g., stent portions) to adhere device 500 to the blood vessel tissue, as shown at anchor substitution portions 518 and 520 in FIG. 5A.

FIG. 5C illustrates an example cross-sectional view 550 of a lumen of the example flow modulating device in a constricted configuration. The lumen may be generated within device 500 when assembling the plurality of frame wires 522a-528b to anchors 512a-512c of the first annular member 506, the anchors 514a-514b of the second annular member 508, and/or the anchors 516a-516c of the third annular member. When the device 500 is placed into the constricted configuration, an aperture 552 defined by device 500 may be narrowed by bending one or more frame wires 522a-528b to move the intermediate anchors 514a, 514b, and 514c, etc. toward one another.

FIG. 5D illustrates an example cross-sectional view of a lumen 556 of the example flow modulating device in an unconstricted configuration. When the device 500 is placed into the unconstricted configuration, an aperture 554 defined by the device 500 may be widened by straightening (or substantially straitening) one or more frame wires 522a-528b to move the intermediate anchors 514a, 514b, and 514c, etc. away one another.

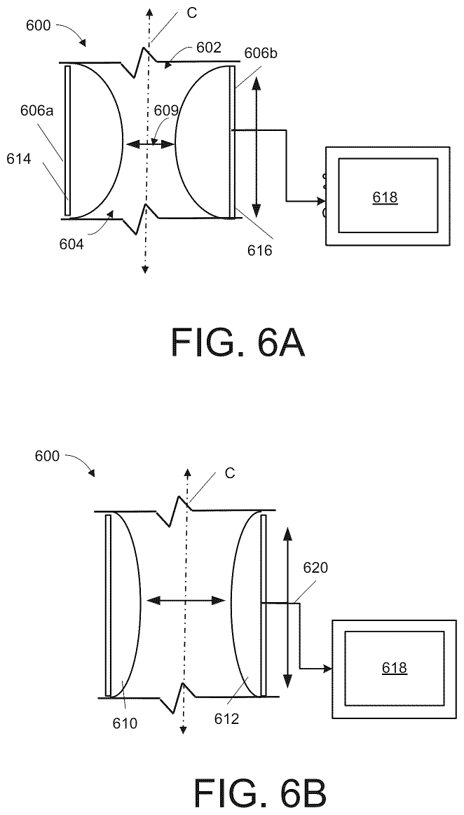

FIGS. 6A-6B illustrate perspective views of an example flow modulating device 600 for modulating blood flow through a blood vessel. The device 600 may be implanted into a blood vessel, such as the superior vena cava, the inferior vena cava, or any other blood vessel where modulating blood flow is desired. For example, the device 600 may modulate a volume of blood flowing from the superior vena cava into a right atrium to decrease right atrial pressure.

At a high level, the device may include a self-expanding or balloon-expandable frame (e.g., stent) that may be delivered into the blood vessel (e.g., via jugular access, subclavian access, or transfemoral access) using a sheathed catheter (not shown). The frame may include or be coupled to one or more control elements associated with one or more membranes. When the one or more control elements are activated, the one or more membranes may expand or contract within the blood vessel to function as an adjustable blood flow restrictor in response to receiving a signal from an actuator.

FIG. 6A illustrates a top-down view of the example implantable device 600 for modulating blood flow through a blood vessel. In this example, the device 600 is shown in a restricted blood flow state (e.g., a constricted configuration). The restricted blood flow state may represent a state of device 600 in which both an inflow 602 and an outflow 604 are partially open to receive fluid (e.g., blood, drugs, saline, etc.) from the inflow 602 and through a device lumen, defined about central axis (C), and through to the outflow 604.

The device 600 includes an expandable frame 606 having a proximal end (e.g., at inflow 602) and a distal end (e.g., at outflow 604) and a longitudinal axis extending therethrough (parallel to central axis (C)). In the depicted example, the frame 606 includes a left frame portion 606a and a right frame portion 606b. However, the frame 606 may instead be formed to surround or partially surround other features of the device 600. The frame 606 defines a lumen from the inflow 602 to the outflow 604 about central axis (C).

The frame 606 may be a stent comprising or constructed of metal (e.g., stainless steel, platinum, Nitinol® wire or another shape memory alloy or polymer) or other material suitable for implantation in the human body. In some examples, the expandable frame 606 is a bare metal stent, such that the expandable frame 606 is configured to be at least partially incorporated into an inner wall of the blood vessel. In some examples, the frame 606 has a pro-endothelialization coating, such that the frame 606 can be at least partially incorporated into an inner wall of the blood vessel. This incorporation may allow a site of the device 600 to maintain a non-thrombogenic, non-immunogenic environment with respect to the device 600. For example, the coating of the frame 606 may be any pro-endothelial factor including, but not limited to, endothelial growth factor, vascular endothelial growth factor, or any related compound.

After the device 600 is implanted, partially implanted, or otherwise incorporated into the blood vessel wall (i.e., tissue may heal around at least a portion of the frame), the frame 606 becomes incorporated into the inner wall of the blood vessel such that contracting or expanding one or more portions of the device 600 has the effect of partially occluding or un-occluding the vessel wall/cross-section, as shown by arrow 609.

The device 600 further includes a first membrane portion 610 and a second membrane portion 612. The first membrane portion 610 may be coupled to a first internal portion 614 of the frame 606a. For example, the first membrane portion 610 may be affixed or otherwise adhered to the first internal portion 614 along longitudinal lines or points via adhesive, mechanical fasteners, or the like. The second membrane portion 612 may be affixed or otherwise adhered to the second internal portion 616 along longitudinal lines or points via adhesive, mechanical fasteners, or the like. The first internal portion 614 is opposite the second internal portion 616.

The membrane portions 610, 612 may be elastic to expand and contract. In some examples, the membrane portions 610, 612 are dome shaped. In some examples, the membrane portions 610, 612 are hemisphere shaped. In some examples, the membrane portions 610, 612 are rectangular shaped.

In general, when the membrane portions 610, 612 are expanded, the motor 618 may cause the frame portions 606a, 606b to shorten longitudinally such that membrane portions 610, 612 expand inward toward the central axis (C) as the frame portions 606a, 606b are shortened. Similarly, when the membrane portions 610, 612 are contracted, the motor 618 may cause the frame portions 606a, 606b to lengthen longitudinally such that membrane portions 610, 612 contract outward away from the central axis (C). The expansion and contraction of membrane portions 610, 612 may be performed in such a manner because frame portions 606a, 606b are formed from a shape memory which may allow lengthening and shortening of the frame portions 606a, 606b, in response to an input (e.g., mechanical signal or electrical signal) received at the portions 606a, 606b. In some examples, membrane portions 610, 612 are expandable and contractible based on expanding and contracting the actual material of portions 610, 612, similar to a medical balloon.

The membrane portions 610, 612 may be formed of thermoplastic elastomeric and/or non-elastomeric polymers. Example suitable materials for the membrane portions 610, 612 may include, but are not limited to: polyesters, polyolefins, polyurethanes, polyimides, polyamides, polycarbonates, polyethers, silicones, or copolymers and/or mixtures thereof. The first membrane portion 610 and/or the second membrane portion 612 may be expanded toward the central axis (C) of the expandable frame 606 to cause a narrowing of the lumen. For example, the first membrane portion 610 and/or the second membrane portion 612 may expand and contract to regulate blood flow through the blood vessel. Expanding the first membrane portion 610 or the second membrane portion 612 within the expandable frame 606 may result in partially occluding the blood vessel. Performing a partial occlusion of the blood vessel may include blocking about 50 percent to about 80 percent of the blood flowing through the blood vessel.

Expanding both the first membrane portion 610 within the expandable frame 606 and the second membrane portion 612 within the expandable frame 606 may result in fully occluding the blood vessel. Performing a full occlusion of the blood vessel may include blocking about 80 percent to about 100 percent of the blood flowing through the blood vessel resulting in about 2 mmHG to about 15 mmHg.

The device 600 also includes an actuator in the form of a motor 618. The motor 618 may be coupled to either or both of the first membrane portion 610 or the second membrane portion 612. The motor 618 may be engaged to reversibly expand one or both of the first membrane portion 610 or the second membrane portion 612 to reduce blood flow through the blood vessel. In some examples, the motor 618 may reversibly expand one or both of the first membrane portion 610 or the second membrane portion 612 to stop blood flow through the blood vessel.

In some examples, the motor 618 is a linear actuator coupled to the first membrane portion 610 and the second membrane portion 612. The linear actuator may be, for example, a pneumatic linear actuator, an electromechanical linear actuator, or a hydraulic linear actuator. The linear actuator may be used to expand or contract the first membrane portion 610 and/or the second membrane portion 612 to put the device 600 into a restricted blood flow state or an unrestricted blood flow state, respectively.

FIG. 6B illustrates the device 600 in an unrestricted blood flow state (e.g., an unconstricted configuration). The unrestricted blood flow state may represent a state of device 600 in which both the inflow 602 and the outflow 604 are open to receive fluid (e.g., blood, drugs, saline, etc.) through the device lumen, about central axis (C). For example, the first membrane portion 610 and the second membrane portion 612 may be in the unrestricted blood flow state when the portions 610, 612 are contracted, as shown in FIG. 6B

Contracting the first membrane portion 610 within the expandable frame 606 and contracting the second membrane portion 612 within the expandable frame 606 may result in restoring blood flow through the blood vessel. Contracting the first membrane portion 610 or contracting the second membrane portion 612 within the expandable frame 606 may result in partially restoring blood flow through the blood vessel.

In some examples, the motor 618 may be coupled to a mechanical actuator for expanding and/or contracting the first membrane portion 610 or the second membrane portion 612. For example, the mechanical actuator may be a control wire 620 connected to one or both of the first membrane portion 610 or the second membrane portion 612. Expanding of the first membrane portion 610 or the second membrane portion 612 may be performed in response to actuation of the control wire 620. The actuation of the control wire 620 may be triggered by the motor 618. Similarly, contracting of the first membrane portion 610 or the second membrane portion 612 may be performed in response to actuation of the control wire 620 using the motor.

In some examples, the device 600 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator (e.g., control wire 620), the microprocessor 1008, and the sensor 1006. For example, the sensor 1006 may sense characteristics of blood flow in the blood vessel (e.g., blood pressure) and may cause the microprocessor to provide signals to the actuator (e.g., control wire 620 or actuation device 1012). In operation, the microprocessor 1008 can receive a signal from the sensor 1006 that is indicative of a pressure in the blood vessel. The microprocessor 1008 can process the signal and generate and provide a control signal (e.g., via control devices 1010) to expand membrane portions 610, 612, or contract membrane portions 610, 612 based on the sensed pressure in the blood vessel. For example, the microprocessor 1008 may receive a signal from the sensor in which the signal is indicative of a pressure in the blood vessel. The microprocessor 1008 may then output a control signal to the linear actuator (e.g., motor 618) to either expand the first membrane portion 610 or contract the first membrane portion 610 based on the sensed pressure in the blood vessel. The microprocessor may alternatively, or additionally, output a control signal to the linear actuator (e.g., motor 618) to either expand the second membrane portion 612 or contract the second membrane portion 612 based on the sensed pressure in the blood vessel.

In some examples, the sensor 1006 may be communicatively coupled to device 600. The sensor 1006 may include one or more of an image sensor, a strain gauge, a piezoelectric sensor, a capacitance sensor, and/or a vacuum pressure sensor. If the device 600 is coupled to a power source (e.g., power source 1014), the power source may include an induction coil in the event that the power source includes magnets and/or coils. The induction coil may be used to operate one or more of such magnets, as described in further detail in FIGS. 11A-11B.

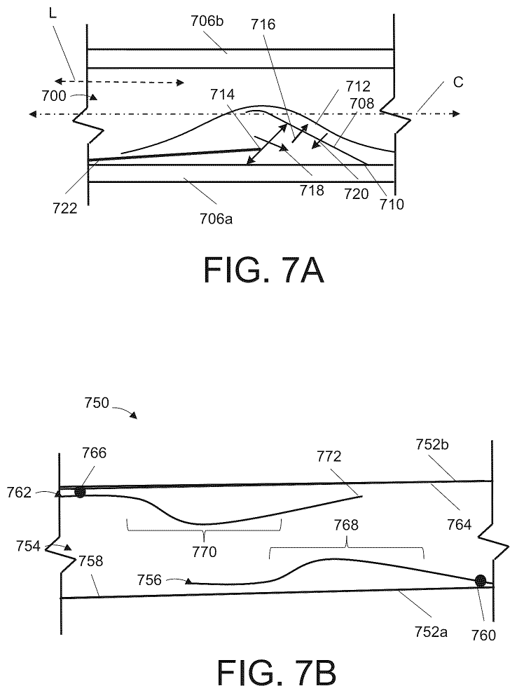

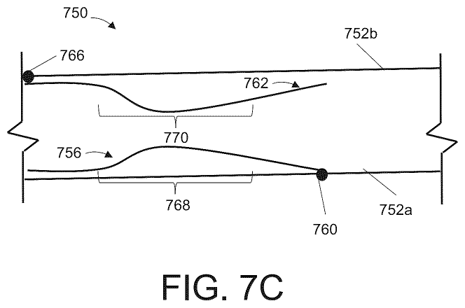

FIGS. 7A-7C illustrate side views of example flow modulating devices for modulating blood flow through a blood vessel. The devices may be implanted into a blood vessel, such as the superior vena cava, the inferior vena cava, or any other blood vessel where modulating blood flow is desired. For example, the implantable devices may modulate a volume of blood flowing from the superior vena cava into a right atrium to decrease right atrial pressure. Such devices may include one or more components that may move within a stream of blood flow to modulate the blood flow in a blood vessel in which the devices are implanted.

FIG. 7A is a side view of an example flow modulating device 700 for modulating blood flow through a blood vessel. The device 700 includes a frame 706 having a first portion 706a and a second portion 706b. The frame portions 706a, 706b may comprise or be formed of one or more of: a polymer, a biomaterial, or a textile. The frame portions 706a, 706b define a lumen in which blood may flow when the device 700 is implanted in a blood vessel. In general, the device 700 may be transitionable between a constricted configuration and an unconstricted configuration.

The device 700 includes a lever arm 708 coupled to an inner surface portion 710 of the frame portion 706a. The lever arm 708 may be formed of a shape memory alloy or polymer.

The lever arm 708 may be at least partially covered by a tissue portion 712. The tissue portion 712 may be an elasticized tissue.

The device 700 also includes a control element 714. The control element 714 may be disposed between the frame portion 706a and the lever arm 708. The control element 714 may be configured to move the lever arm 708 from a first position to a second position. For example, the first position may include the lever arm 708 being substantially parallel to a longitudinal axis (L) defined by the frame. The second position may include the lever arm 708 being raised to an acute angle from an axis perpendicular to the longitudinal axis (L). The second position may cause the control element 714 to engage the tissue portion 712 and move the tissue portion 712 toward a central axis (C) of the lumen, in the direction of arrow 716.

The device 700 may also transition from the second position to the first position thereby causing the control element 714 to contract or move toward the frame portion 706a to lower the lever arm 708 in the direction of arrow 718, and thus lower the tissue portion 712, in the direction of arrow 720.

The device 700 may also include a mechanical actuator 722. The mechanical actuator 722 may cause the control element 714 to transition the device 700 from the unconstricted configuration to the constricted configuration by moving the lever arm 708 from the first position to the second position. The mechanical actuator 722 may also cause the control element 714 to transition the device 700 from the constricted configuration to the unconstricted configuration by moving the lever arm 708 from the second position to the first position.

In some examples, the lever arm 708 may elongate to raise the tissue portion 712 when moving from the first position to the second position, in response to receiving a first actuation signal from the mechanical actuator 722. For example, the first actuation signal may be received from the mechanical actuator 722 to trigger the tissue portion 712 to move to the constricted configuration.

In some examples, the lever arm 708 may be triggered to contract to lower the lever arm from the second position to the first position, in response to receiving a second actuation signal from the mechanical actuator. For example, the second actuation signal triggers the tissue portion 712 to move to the unconstricted configuration. In some examples, the mechanical actuator 722 is configured to tension the control element 714 to transition the device to the constricted configuration and to release the control element 714 to transition the device 700 (and/or tissue portion 712) to the unconstricted configuration.

In some examples, the device 700 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator 722, the microprocessor 1008, and the sensor 1006. For example, the sensor 1006 may sense characteristics of blood flow in the blood vessel (e.g., blood pressure) and may cause the microprocessor to provide signals to the actuator 722 (e.g., actuation device 1012) and/or control element 714 (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to contract and/or raise the lever arm 708 from the first position to the second position, or to relax and/or elongate the control element 714 based on the sensed pressure in the blood vessel.

In some examples, the sensor 1006 and/or microprocessor 1008 may output a control signal to the mechanical actuator 722 to either tension the control element 714 or release tension in the control element 714 based on the sensed pressure in the blood vessel. In some examples, a tensioned control element 714 may raise the lever arm (and in turn the tissue portion 712) to restrict blood flow through the blood vessel.

In some examples, the sensor 1006 may be communicatively coupled to device 700. The sensor 1006 may include one or more of an image sensor, a strain gauge, a piezoelectric sensor, a capacitance sensor, and/or a vacuum pressure sensor. If the device 700 is coupled to a power source (e.g., power source 1014), the power source may include an induction coil in the event that the power source includes magnets and/or coils. The induction coil may be used to operate one or more of such magnets, as described in further detail in FIGS. 11A-11B.

FIG. 7B is a side view of an example flow modulating device 750 for modulating blood flow through a blood vessel. The device 750 includes a frame 752 having a first portion 752a and a second portion 752b. The frame 752 defines a lumen 754 in which blood may flow when the device 750 is implanted in a blood vessel. In general, the device 750 may be transitionable between a constricted configuration and an unconstricted configuration.

The device 750 may include a first insert 756 that is at least partially attached to a first interior wall 758 of the expandable frame portion 752a, as shown at junction 760. The first insert 756 may represent a first wire within the expandable frame 752. The first wire (e.g., first insert 756) may be formed in an S-curve shape and may be composed of a shape memory alloy or polymer. In some examples, the first insert 756 may instead be a first surface or structure that is formed as a three-dimensional component.

The device 750 may include a second insert 762 that is at least partially attached to a second interior wall 764 of the expandable frame portion 752b, as shown at junction 766. The first interior wall 758 is opposite the second interior wall 764. The second insert 762 may represent a second wire within the expandable frame 752. The second wire (e.g., second insert 762) may be formed in an inverted S-curve shape and may be composed of a shape memory alloy or polymer. In some examples, the second insert 762 may instead be a surface or structure that is formed as a three-dimensional component.

The device 750 further includes a mechanical actuator to tension the first insert 756 or the second insert 762 to transition the expandable frame 752 to the constricted configuration. The first insert 756 (e.g., wire) may include an intermediate portion 768 that may be aligned with an intermediate portion 770 of the second insert 762 (e.g., wire) to change a state of device 700. For example, the mechanical actuator may slide the first insert 756 to transition along the first interior wall 758 from a predefined position (e.g., shown in FIG. 7B) to the constricted configuration (e.g., shown in FIG. 7C). Sliding the first insert 756 along the wall 758 may result in substantially aligning the intermediate portion 768 of the first wire with an intermediate portion 770 of the second wire (e.g., insert 762). The alignment of the intermediate portion 770 with the intermediate portion 768 may provide at least a partial occlusion of the blood vessel.

The mechanical actuator may also release tension in the first insert 756 or the second insert 762 to transition the expandable frame 752 to the unconstricted configuration. For example, the mechanical actuator may slide the first insert 756 to transition along the first interior wall 758 to the unconstricted configuration by sliding the first insert 756 to the predefined position to align the intermediate portion 768 of the first wire (e.g., first insert 756) with an end portion 772 of the second wire (e.g., second insert 762). In some examples, the predefined configuration is the unconstricted configuration.

In some examples, the device 750 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the mechanical actuator, the microprocessor 1008, and the sensor 1006. For example, the sensor 1006 may sense characteristics of blood flow in the blood vessel (e.g., blood pressure) and may cause the microprocessor to provide signals to the mechanical actuator (e.g., actuation device 1012) and/or control element (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to slide the first insert 756 from the predefined position to the constricted configuration (e.g., FIG. 7C) or return the first insert 756 to the predefined position based on the sensed pressure in the blood vessel. Similarly, the microprocessor 1008 can process the signal and generate and provide a control signal (e.g., via control devices 1010) to slide the second insert 762 from a predefined position to the constricted configuration or return the second insert 762 to the predefined position based on the sensed pressure in the blood vessel.

In some examples, the sensor 1006 may be communicatively coupled to device 750. The sensor 1006 may include one or more of an image sensor, a strain gauge, a piezoelectric sensor, a capacitance sensor, and/or a vacuum pressure sensor. If the device 750 is coupled to a power source (e.g., power source 1014), the power source may include an induction coil in the event that the power source includes magnets and/or coils. The induction coil may be used to operate one or more of such magnets, as described in further detail in FIGS. 11A-11B.

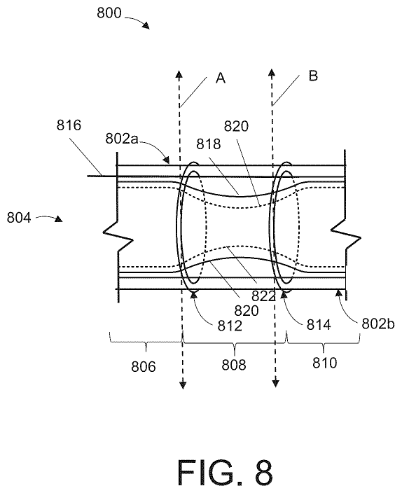

FIG. 8 is a side view of an example flow modulating device 800 for modulating blood flow through a blood vessel. The device 800 may be implanted into a blood vessel, such as the superior vena cava, the inferior vena cava, or any other blood vessel where modulating blood flow is desired. For example, the implantable device 800 may modulate a volume of blood flowing from the superior vena cava into a right atrium to decrease right atrial pressure.

At a high level, the device may include a self-expanding or balloon-expandable frame (e.g., stent) that may be delivered into the blood vessel (e.g., via jugular access, subclavian access, or transfemoral access) using a sheathed catheter (not shown). The frame may include or be coupled to at least two flexible control elements. When tension is applied to the control elements, a lumen defined by the frame may be circumferentially reduced forming a blood flow restricting orifice. The frame may expand and contract within the blood vessel using tension to function as an adjustable blood flow restrictor.

The device 800 includes an expandable frame 802 having a first portion 802a and a second portion 802b. The frame 802 defines a lumen 804 therethrough. The frame 802 may be transitionable between a constricted configuration and an unconstricted configuration. The constricted configuration may reduce blood flow through the lumen. The unconstricted configuration may allow blood to flow through the lumen substantially unencumbered.

In some examples, the frame 802 may be a stent comprising metal (e.g., stainless steel, platinum, Nitinol® wire or another shape memory alloy or polymer) or other material suitable for implantation in the human body. In some examples, the expandable frame 802 is a bare metal stent, such that the expandable frame 802 is configured to be at least partially incorporated into an inner wall of the blood vessel. In some examples, the expandable frame 802 has a pro-endothelialization coating, such that the expandable frame 802 can be at least partially incorporated into an inner wall of the blood vessel. This incorporation may allow a site of the device 800 to maintain a non-thrombogenic, non-immunogenic environment with respect to the device 800. For example, the coating of the frame 802 may be any pro-endothelial factor including, but not limited to, endothelial growth factor, vascular endothelial growth factor, or any related compound.

After the device 800 is implanted and incorporated into the blood vessel wall (i.e., tissue may heal around at least a portion of the frame), the frame 802 becomes incorporated into the inner wall of the blood vessel such that cinching or pinching a portion of the device 800 has the effect of also cinching/pinching the vessel wall/cross-section at (or adjacent to) a cinching/pinching location corresponding to the portion of the device 800. Optionally, the frame 802 may be wrapped with a membrane that can form a seal between an outer surface portion (e.g., an outer diameter) of the frame 802 and the inner wall of the blood vessel. The formed seal may prevent leakage in-between the device 800 and the blood vessel inner wall when the cinching occurs.

Referring again to FIG. 8, the frame 802 includes a first end portion 806, an intermediate portion 808, and a second end portion 810. The first end portion 806 may represent an inflow for device 800 adjacent to an interface A of the intermediate portion 808. The second end portion 810 may represent an outflow for device 800 that interfaces with an interface B. In some examples, the first end portion 806, the intermediate portion 808, and the second end portion 810 are formed as a single device portion, and are identified separately to indicate different areas of movement throughout the device 800. Interfaces A and B are not seams or connection interfaces, but are shown for clarity and differentiation amongst the first end portion 806, the intermediate portion 808, and the second end portion 810.

The device 800 may include a first control element 812. The first control element 812 may be at least partially circumferentially disposed about the expandable frame 802. In this example, the first control element 812 is depicted as a substantially annular wire wrapped at least partially around the frame 802 such that when the control element 812 is tensioned, an internal area within the frame 802 may be reduced in at least one cross section of the lumen of the frame 802.

The device 800 also includes a second control element 814. The second control clement 814 may be at least partially circumferentially disposed about the expandable frame 802. The second control element 814 is depicted as a substantially annular wire wrapped at least partially around the frame 802 such that when the control element 814 is tensioned, an internal surface area within the frame 802 may be reduced in at least one cross section of the lumen of the frame 802.

Other shapes, materials, and/or structures for control elements are possible that contemplate at least partially encompassing device 800 to constrict and reduce a portion of frame 802 and thus constrict a portion of device 800. In some examples, at least one of the first control element 812 or the second control element 814 are formed of a shape memory alloy (e.g., Nitinol® or a combination of Nitinol® and palladium, gold, or their alloys or polymer).

In some examples, the first control element 812 is at least partially circumferentially disposed about a first location on the intermediate portion 808 of the expandable frame 802. For example, the first location may be positioned at the interface A of the first end portion 808. In some examples, the second control element 814 is at least partially circumferentially disposed about a second location on the intermediate portion 808 of the expandable frame 802. For example, the second control element 814 may be positioned at the interface B of the second end portion 810.

The device 800 may also include a mechanical actuator 816 for tensioning the first control element 812 and/or the second control element 814 to transition the expandable frame 802 to the constricted configuration. For example, the mechanical actuator 816 may tension the first control element 812 to transition the element at the first location 818 on the intermediate portion 808 of the expandable frame 802 to the constricted configuration (e.g., as shown by the element at location 820). Further, the mechanical actuator 816 may release tension on the first control element 812 to release tension in the first control element 812 or the second control element 814 to transition the expandable frame 802 to the unconstricted configuration, as shown by the element at the location 818.

Similarly, the mechanical actuator 816 may tension the second control element 814 to transition the element at the second location 820 on the intermediate portion 808 of the expandable frame 802 to the constricted configuration, as shown by dotted line 822. The mechanical actuator 816 may also release tension in the second control element 814 to transition the device to the unconstricted configuration, as shown by the element at the location 820.

In some examples, tensioning both the first control element 812 and the second control element 814 may result in occluding the blood vessel. For example, tensioning both control elements 812, 814 may function to fully occlude a portion of the device lumen and thus fully occluding the blood vessel in which the device 800 is implanted. In some examples, tensioning either the first control element 812 or the second control element 814 may result in partially occluding a portion of the device lumen and thus partially occluding the blood vessel in which the device 800 is implanted.

In some examples, the mechanical actuator 816 is a linear actuator. The linear actuator may be, for example, a pneumatic linear actuator, an electromechanical linear actuator, or a hydraulic linear actuator. The linear actuator may be used to tension or release tension in the control clement 812 and/or the control element 814 to put the device 800 into a restricted blood flow state or an unrestricted blood flow state, respectively.

In some examples, the device 800 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the actuator 816, the microprocessor 1008, and the sensor 1006. For example, the sensor 1006 may sense characteristics of blood flow in the blood vessel (e.g., blood pressure) and may cause the microprocessor to provide signals to the actuator 816 (e.g., actuation device 1012) and/or control elements 812, 814 (e.g., control devices 1010). In operation, the microprocessor 1008 can receive a signal from the sensor 1006 that is indicative of a pressure in the blood vessel. The microprocessor 1008 can process the signal and generate and provide a control signal (e.g., via control devices 1010) to tension the control elements 812, 814, or release tension in the control elements 812, 814 based on the sensed pressure in the blood vessel.

In some examples, the sensor 1006 may be communicatively coupled to device 800. The sensor 1006 may include one or more of an image sensor, a strain gauge, a piezoelectric sensor, a capacitance sensor, and/or a vacuum pressure sensor. If the device 800 is coupled to a power source (e.g., power source 1014), the power source may include an induction coil in the event that the power source includes magnets and/or coils. The induction coil may be used to operate one or more of such magnets, as described in further detail in FIGS. 11A-11B.

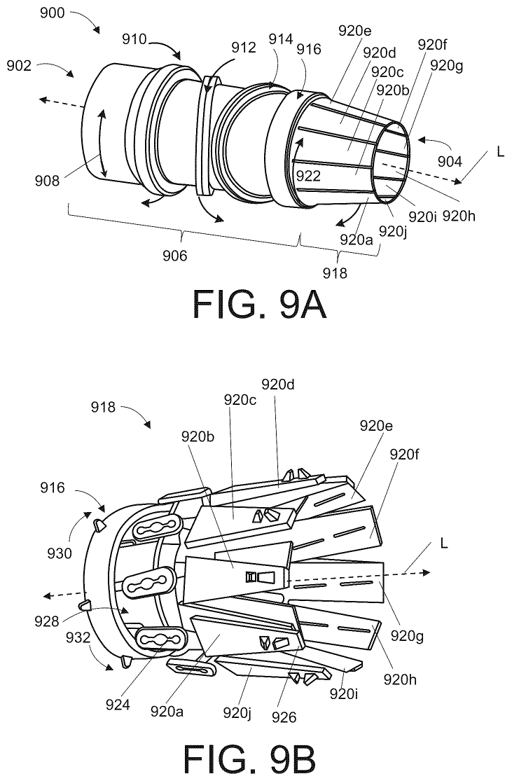

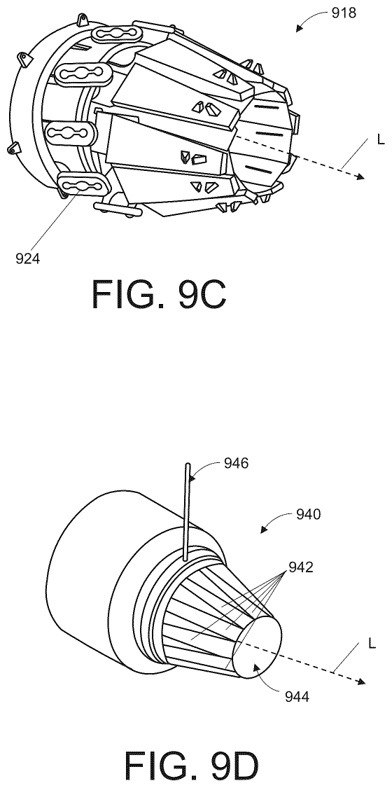

FIGS. 9A-9D illustrate a side view of an example flow modulating device 900 for modulating blood flow through a blood vessel. The device 900 may be implanted into a blood vessel, such as the superior vena cava, the inferior vena cava, or any other blood vessel where modulating blood flow is desired. For example, the device 900 may modulate a volume of blood flowing from the superior vena cava into a right atrium to decrease right atrial pressure.

At a high level, the device 900 may include a nozzle coupled to a control element to radially collapse or expand a plurality of projections coupled to the end of the nozzle. Actuating the plurality of projections may function to occlude or partially occlude the blood vessel in which device 900 is implanted.

FIG. 9A illustrates a perspective view of the example device 900 for modulating blood flow through a blood vessel. In this example, the device 900 is shown in a partially restricted blood flow state (e.g., a partially constricted configuration). The partially restricted blood flow state may represent a state of device 900 in which an inflow (e.g., at a proximal end 902) is open to receive fluid (e.g., blood, drugs, saline, etc.) while an outflow (e.g., at a distal end 904) is partially constricted and thus partially blocking blood flow through the blood vessel when the device 900 is implanted in a blood vessel.

The device 900 includes a housing 906. The housing 906 includes the proximal end 902 and the distal end 904 and a longitudinal axis (L) extending therethrough. The housing 906 may include two or more sections nested together to allow twisting of the sections in a clockwise radial direction and a counterclockwise radial direction, as indicated by arrow 908. For example, a first section 910, a second section 912, a third section 914, and a fourth section 916 may be rotated to modify blood flow through device 900 and thus through a blood vessel in which device 900 is implanted. The first section 910, the second section 912, the third section 914, and the fourth section 916 may be rotated in response to receiving one or more signals from an actuation device, as described elsewhere herein. In some examples, the first section 910, the second section 912, the third section 914, and/or the fourth section 916 function as a control element coupled to a portion of the housing 906. For example, any one of sections 910-916 may be a separate member that is coupled to another portion of the housing 906 when generating the assembly of device 900. The housing 906 is coupled to a nozzle 918. The nozzle 918 forms the distal end 904 of the device 900. In some examples, the fourth section 916 functions as a distal end (e.g., at section 916) of the housing 906. The distal end of the housing 906 may attach to the nozzle 918.

FIG. 9B illustrates a perspective view of the nozzle 918 installed in the device 900. The nozzle 918 may function to fully restrict a flow of blood resulting in occlusion of the blood vessel in which device 900 is implanted. The nozzle 918 may also function to partially restrict the flow of blood resulting in a partial occlusion of the blood vessel in which device 900 is implanted.

The nozzle 918 may be opened, closed, and in various partially opened states in between open and closed. As shown in FIG. 9A, the nozzle 918 is partially opened for modifying the flow of blood through device 900. For example, the nozzle 918 includes a plurality of elongate projections 920a, 920b, 920c, 920d, 920c, 920f, 920g, 920h, 920i, and 920j. The elongate projections 920a-920j are arranged radially around an outer surface 922 (FIG. 9A) of the distal end (e.g., at section 916) of the housing 906. The elongate projections 920a-920j extend substantially parallel to the longitudinal axis (L).

Each elongate projection 920a-920j includes a first end portion (e.g., end portion 924) that is coupled to the distal end (e.g., at section 916) of the housing 906. Each elongate projection 920a-920j includes a second end portion (e.g., end portion 926) that forms a portion of a nozzle 918 outflow located at the distal end 904 of the device 900. Other end portions corresponding to elongate projections 920b-920j are not labeled with a first end portion or a second end portion for purposes of clarity of the figure, but one skilled in the art would appreciate that each projection 920b-920j also include a first end portion and a second end portion similar to projection 920a.

In operation, the elongate projections 920a-920j are capable of radially collapsing at the respective second end portions (e.g., end portion 926, etc.) and toward a central axis of the housing (e.g., parallel to longitudinal axis (L)) in response to actuation of the control element (e.g., actuation of one or more of sections 910-916). For example, the elongate projections 920a-920j may individually collapse toward longitudinal axis (L) to narrow an outflow of the nozzle 918 (as shown in FIG. 9C). In addition, the nozzle 918 may be projected directionally as one or more of the elongate projections 920a-920j are moved perpendicularly away from a longitudinal axis (L). For example, one or more of the projections 920a-920j can move latitudinally (in a latitudinal plane) perpendicular to a longitudinal axis. Further for example, one or more of the projections 920a-920j can move in a longitudinal plane away from the longitudinal axis (L). One or more projections 920-902j may also move at an angle between the longitudinal plane and a latitudinal plane. In some examples, one or more of the elongate projections 920a-920j may radially collapse while others of the elongate projections 920a-920j remain fixed or unmoving. In some examples, each of the elongate projections 920a-920j can simultaneously begin to radially collapse, moving together to another position or narrowed state of the nozzle.

The elongate projections 920a-920j are also capable of radially expanding away from the central axis (e.g., parallel to longitudinal axis (L)) of the housing at the respective second end portions (e.g., end portion 926, etc.), in response to actuation of the control element (e.g., actuation of one or more of sections 910-916). For example, the elongate projections 920a-920j may individually expand away from longitudinal axis (L) to partially or fully expand (e.g., open) the nozzle 918. In some examples, one or more of the elongate projections 920a-920j may radially expand while others of the elongate projections 920a-920j remain fixed or unmoving. In some examples, each of the elongate projections 920a-920j can simultaneously begin to radially expand, moving together to another position or expanded state of the nozzle.

In some examples, the elongate projections 920a-920j are coupled to a housing base 928. The elongate projections 920a-920j may radially expand or radially collapse in response to actuation of the control element (e.g., section 916). For example, actuating the control element includes (or may result in) twisting the housing base 928 (and/or twisting section 916 or another section 910-914) in a first direction 930 (e.g., to radially expand the plurality of elongate projections to allow blood flow through the blood vessel.

In some examples, the device 900 may be re-actuated after an initial (or prior) actuation is performed. For example, the control element (e.g., section 916) may be re-actuated to cause the housing base 928 to twist in a second direction 932 to radially collapse the elongate projections 920a-920j to reduce blood flow through the blood vessel.

In some examples, the elongate projections 920a-920j are wedge-shaped. The wedge shape may allow the base of the second end portions (e.g., 926, etc.) of each of the elongate projections 920a-920j to be broad to allow for full blood flow near the base while allowing the end of the nozzle 918 (e.g., the second end portion 926, etc.) to be tapered to partially occlude or fully occlude flow through the distal end 904 of the device 900.

In some examples, the elongate projections 920a-920j are triangular shaped. In some examples, the elongate projections 920a-920j rectangular-shaped. In some examples, the elongate projections 920a-920j are polygon-shaped.

In some examples, the device 900 may be fitted with any number of nozzle types. FIG. 9D is an example nozzle 940 that may be coupled to device 900. For example, a nozzle 940 may replace nozzle 918. The nozzle 940 includes a number of elongate projections 942, similar to elongate projections 920a-920j in that the projections 942 may function to partially expand or partially collapse to open, close, or partially open the nozzle 940. As shown, the nozzle 940 is partially opened with an outflow end 944 allowing a portion of fluid to flow through the nozzle 940. In this example, a control element 946 may be used to radially contract or radially expand the elongate projections 942 to put the device 900 (attached to nozzle 940) into a restricted blood flow state or an unrestricted blood flow state, respectively.

In some examples, the device 900 may further include a sensor (e.g., sensor 1006 of FIG. 10) for detecting a pressure in the blood vessel, a microprocessor (e.g., processor 1008) electrically coupled to the sensor 1006, and/or a power source (e.g., power source 1014) electrically coupled to the microprocessor 1008, the sensor 1006, and/or an actuator for actuating device 900. For example, the sensor 1006 may sense characteristics of blood flow in the blood vessel (e.g., blood pressure) and may cause the microprocessor to provide signals to the actuator (e.g., actuation device 1012) and/or control element 1010 (e.g., control elements/sections 910, 912, 914, 916, and/or 946). In operation, the microprocessor 1008 can receive a signal from the sensor 1006 that is indicative of a pressure in the blood vessel. The microprocessor 1008 can process the signal and generate and provide a control signal (e.g., via control devices 1010 or the like) to tension the control element, or release tension in the control element based on the sensed pressure in the blood vessel.

In some examples, the sensor 1006 may be communicatively coupled to device 900. The sensor 1006 may include one or more of an image sensor, a strain gauge, a piezoelectric sensor, a capacitance sensor, and/or a vacuum pressure sensor. If the device 900 is coupled to a power source (e.g., power source 1014), the power source may include an induction coil in the event that the power source includes magnets and/or coils. The induction coil may be used to operate one or more of such magnets, as described in further detail in FIGS. 11A-11B.

In some examples, the device 900 may receive a mechanical actuation from a mechanical actuator (e.g., actuation device 1108 of FIGS. 11A-11B or actuation devices 1012 of FIG. 10) to cause the nozzle 918 (or nozzle 940) to expand or contract (e.g., collapse) radially.

Although a frame and/or stent is not shown to surround each of the devices described herein, one skilled in the art would contemplate providing a frame and/or stent surrounding such devices for purposes of enabling the devices to be implanted and secured to a blood vessel wall. For example, devices depicted in FIGS. 3A-3D, 5A-5B, 6A-6B, 7A-7C, 8, and 9A-9D are depicted without a frame and/or stent surround, but such devices may function inside of a frame or stent; inside of a portion of a frame or stent; and/or without a stent.

Although restricted and unrestricted flow states/configurations or restricted and unrestricted device positions are described herein, it is within the scope of the present disclosure that any number of intermediate positions or states are contemplated and included herein, whether or not expressly indicated.

FIG. 10 is a block diagram of an example system 1000 for modulating blood flow through one or more blood vessels. The system 1000 may be used with any of the flow restricting devices described herein. As shown, the system 1000 includes flow restriction controls 1002 and at least one implantable device 1004. The implantable device 1004 may correspond to any of the flow restricting devices described herein.

The flow restriction controls 1002 may include one or more optional sensors 1006, one or more processors 1008, one or more control devices 1010, and one or more actuation devices 1012. Optionally, the flow restriction controls may include a power source 1014 that may be internal to the controls 1002, internal to the implantable device 1004, or external to both the flow restriction controls 1002 and the implantable device 1004. In some examples, the power source may be wired to flow restriction controls 1002 or implantable device 1004. In some examples, the power source may be remotely accessed (e.g., wirelessly) by flow restriction controls 1002 or implantable device 1004.

The optional sensors 1006 may generally function to sense (e.g., detect) properties of the blood in which the sensor(s) are disposed within. For example, the optional sensors 1006 may detect blood pressure within the blood vessel and/or any other physiological or anatomical parameters or properties of the blood or vessel. The optional sensors 1006 may include one or more of an image sensor, a strain gauge, a piezoelectric sensor, a capacitance sensor, and/or a vacuum pressure sensor. In general, sensor signals from sensors 1006 may be transmitted to control devices or elements described herein via a wired or wireless connection. Additionally, and optionally, the sensors 1006 may utilize one or more processors 1008 to transmit data to remote computing devices. The transmitted data may include sensor measurements, device position data and/or statistics, actuation events, or any other data from the system.

The processors 1008 may include one or more microprocessors, microcontrollers, or the like, as described elsewhere herein. The control devices 1010 may include active or passive controls including, but not limited to wires, sutures, operated switches, motor controllers, and/or antennas. In some examples, the control devices 1010 may include external control devices including, but not limited to remote computers, tablets, smart phones, and/or external control devices for powering and/or controlling the flow restriction controls 1002.

The actuation devices 1012 may include mechanically actuating devices, electrically actuated devices, electromechanically actuated devices, or a combination thereof. For example, actuation devices 1012 may include any one or more of a wire, a suture, a pull wire, a linear actuator (e.g., a pneumatic linear actuator, an electromechanical linear actuator, or a hydraulic linear actuator), a magnet or coil, etc. The power sources 1014 may include, but are not limited to, battery power, wall power, magnets, induction coils, or the like.

In operation of system 1000, the actuation device 1012 may be coupled to the control device 1010, which may manipulate or move portions of the implantable device 1004 based on one or more signals received from a sensor 1006. In embodiments that utilize a processor 1008, the processor 1008 may be communicatively coupled to sensors 1006, control devices 1010, actuation devices 1012, power source 1014, and/or implantable device 1004 to actuate the implantable device 1004 into a restricted blood flow state, an unrestricted blood flow state, or any position therebetween.

FIG. 11A is a schematic diagram of an example embodiment of a system 1100 for modulating blood flow through a blood vessel. The system 1100 may include a first magnet 1106, an actuation device 1108, and a control element 1114. The first magnet 1106 may be operatively coupled to the actuation device 1108. The actuation device 1108 may be operatively coupled to the control element 1114 to effect movement of the control element 1114. In some examples, the control element 1114 is a membrane. In some examples, the control element 1114 is a control wire. In some examples, the control element 1114 is a catheter portion. In some examples, the control element 1114 is a ring.

The system may include a control device 1102 operatively coupled to a second magnet 1104. The control device 1102 can include a microprocessor, power source (a battery, a capacitor, wall outlet, or any other suitable power source), antenna, operated switches, and/or any other control devices. The control device 1102 and second magnet 1104 may be located externally, but proximal to a user. In some examples, the control device 1102 and second magnet 1104 may be implanted (e.g., subcutaneously, intravascularly, etc.). In some examples, both the first magnet 1106 and the second magnet 1104 may be permanent magnets. In some examples, the first magnet 1106 is a permanent magnet and the second magnet 1104 is an electromagnet.

Optionally, the system 1100 may include a sensor 1110. The sensor 1110 may sense one or more physiological or anatomical attributes and output a signal to the control device 1102, which may output an activation signal to the actuation device 1108 to tension or release tension in the control clement 1114.

The second magnet 1104, although external to the user or implanted at a second location (the implantable device being at a first location), may be placed operationally proximal to the first magnet 1106. By doing so, the magnetic pole orientation of the second magnet 1104 influences the magnetic pole direction of the first magnet 1106. For example, a magnetic gear train may be generated between the second magnet 1104 and the first magnet 1106, such that when the control device 1102 rotates the second magnet 1104, the first magnet 1106 is rotated in an opposing direction. Rotating the first magnet 1106 induces movement in the actuation device 1108, which tensions or releases tension in the control clement 1114 or moves the control element 1114 to a restricted or unrestricted blood flow state, respectively.

The control device 1102 may receive signals from one or more optional sensors 1110. Such signals may be indicative of characteristics of blood flow in the blood vessel (e.g., blood pressure). For example, when the control device 1102 receives a signal indicative of a measured pressure higher than a predefined level, the control device 1102 can cause the second magnet 1104 to rotate. The rotation of the second magnet 1104 can cause the first magnet 1106 to rotate, which may actuate the actuation device 1108 to move the control element 1114 towards a restricted blood flow state. Further, when the control device 1102 receives a signal indicative of a measured pressure lower than the predefined level, the control device 1102 may cause the second magnet 1104 to rotate in an opposing direction. The rotation of the second magnet 1104 causes the first magnet 1106 to rotate, thereby actuating the actuation device 1108 to move the control clement 1114 into the unrestricted blood flow state.

In general, sensor signals from sensors 1110 may be transmitted to control devices or elements described herein via a wired or wireless connection. Additionally, and optionally, the sensors 1110 may utilize one or more processors to transmit data to remote computing devices. The transmitted data may include sensor measurements, device position data and/or statistics, actuation events, or any other data from the system.

FIG. 11B is a schematic diagram of an example embodiment of a system 1150 for modulating blood flow through a blood vessel. Similar to FIG. 11A, FIG. 11B shows a magnetic gear train for manipulation of a control element 1114 of an implanted device. The control device 1102 and second magnet 1104, as in FIG. 11A, may be located external to the user or implanted (e.g., subcutaneously, intravascularly, etc.). Similar to FIG. 11A, the first magnet 1106, actuation device 1108, control element 1114, and optional sensor 1110 may be implanted within the user. Unlike FIG. 11A, the embodiment of FIG. 11B includes an implanted (in some embodiments, implanted subcutaneously) repeater magnet 1105. This repeater magnet 1105 may be used to extend the operational distance between the second magnet 1104 and the first magnet 1106, as it is implanted at an appropriate position between the two. Additionally, the repeater magnet 1105 may be used to increase the torsional force that can be applied by the magnetic gear train. Further contemplated embodiments may include a repeater module with a second power source operatively coupled to the repeater magnet 1105 and capable of charging and/or powering the rotation of the repeater magnet 1105. Further, the rotation direction of the second magnet 1104 and first magnet 1106 are now the same, not opposing one another as in the embodiment of FIG. 11A. For example, when manipulating the control element 1114 towards the restricted or unrestricted blood flow states, the second magnet 1104 can be rotated in the same direction as the desired direction of the first magnet 1106.

### METHODS

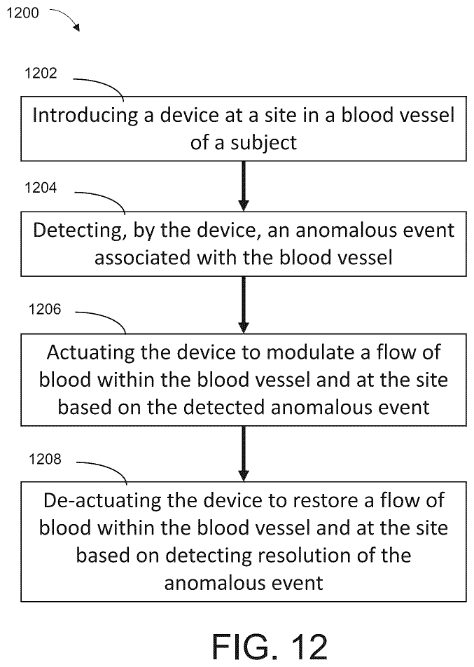

FIG. 12 is a flow diagram of an example process 1200 of modulating blood flow through one or more blood vessels. The process 1200 functions to reduce cardiac blood flow at a target site in a blood vessel of a heart of a subject. In some embodiments, the process 1200 functions to modulate a volume of blood flowing from the blood vessel into a right atrium to decrease right atrial pressure. The process 1200 is used for blood flow regulation in the superior vena cava or inferior vena cava, but can additionally, or alternatively, be used for any suitable applications, clinical or otherwise. In general, process 1200 may be used with any of the devices described in FIGS. 1A-11B above.

As an example, the device used with process 1200 may include device 100 having an expandable frame with a proximal end and a distal end and a longitudinal axis extending therethrough, a membrane with an inflow end and an outflow end. The outflow end may be coupled to a number of elongate support members arranged radially around a surface of the membrane and extending substantially parallel to the longitudinal axis of the frame. The central axis of the expandable frame is substantially parallel to the longitudinal axis. The membrane may be configured to radially collapse at the outflow end and toward a central axis of the expandable frame, or radially expand away from the central axis of the expandable frame, in response to an actuation of a control wire coupled to a portion of the membrane or at least one of the elongate support members. The central axis of the expandable frame is substantially parallel to the longitudinal axis.

In some examples, the process 1200 may be a method of treatment for modulating blood flow in a superior vena cava in a subject having chronic kidney disease and/or chronic heart failure. The method of treatment may include introducing a vessel occlusion device at a site in a blood vessel of a subject (block 1202). For example, the devices described herein may be partially or fully housed by a frame (e.g., a stent). The frame housing the device 100, for example, may be introduced to a vessel or tissue site using a delivery system. In a coronary procedure, a catheter tip and/or catheter may be configured to pass from the right atrium into the coronary sinus to implant the device 100. For access to the venous circulation, for example, a catheter tip and/or catheter may be configured to pass from the radial artery into the superior vena cava to implant the device 100 into a portion of the superior vena cava. Further, for central venous access, a catheter tip and/or catheter may be configured to pass from the femoral vein into the inferior vena cava to implant the device 100 into a portion of the inferior vena cava.

At block 1204, the method of treatment may include detecting, by the device, an anomalous event (or several events) associated with the blood vessel. For example, the process 1200 may include detecting an increasing blood pressure in the right atrium. In particular, the device 100 may include one or more sensors to detect the increased blood pressure and/or the rate of increase. In some examples, the one or more sensors may be configured with a predefined blood pressure threshold level where detecting blood pressure above the predefined blood pressure threshold triggers the device to actuate and lower the blood pressure. In some examples, the predefined threshold may pertain to a rate of pressure increase. In such examples, the one or more sensors may detect that the rate is above the predefined threshold level for rate increase and may cause the device 100 to actuate to modulate a flow of blood within the blood vessel, as shown at block 1206. For example, the device 100 may be actuated based on a sensed anomalous event (or several events). Actuating the device 100 may trigger modulation of a flow of blood within the blood vessel at the device 100 site based on the detected anomalous event. For example, actuating the device 100 may cause a partial occlusion of the blood in the blood vessel at the site of the device. For example, if the device 100 is implanted into the superior vena cava of a subject having chronic kidney disease and/or chronic heart failure, the device can be actuated to modulate a volume of blood flowing from the blood vessel into a right atrium to decrease right atrial pressure.

Further, one or more sensors may be used in conjunction with any of the devices and systems herein to measure one or more physical characteristics of a patient having one of the devices implanted. For example, it may be beneficial to measure whether the patient is standing, sitting, or laying. In addition, the pressure thresholds for activating the device may be influenced by the activity of the patient. For example, it may be beneficial to realize the patient is exercising, as this would elevate pressures and may cause an adjustment in pressure thresholds. Characteristics described above may be measured by a pressure sensor in blood vessels of other portions of the body, a gyroscopic sensor for changes in angular position, an accelerometer for changes in acceleration, a heart rate sensor, a sensor measuring a size of a blood vessel, or any other sensors for measuring physical characteristics. The described characteristics, individually or in combination, may be received by a microprocessor and processed to cause changes in valve position (using an actuating device) based on the sensed characteristics.

At block 1208, the method of treatment may include de-actuating the device to restore a flow of blood within the blood vessel and at the site based on detecting resolution of the anomalous event. For example, when one or more sensors of device 100 detects a resolution of the blood pressure (e.g., the blood pressure is below the threshold level), then the device 100 may trigger a de-actuation of blood flow modulation, which may function to maintain or regain a flow of blood within the blood vessel. Maintaining a flow of blood within the blood vessel may include ensuring the device 100 is held in a particular state of blood regulation such that one or more components of the device may be held stationary over time. Regaining a flow of blood within the blood vessel may include relaxing any blood occlusion components or structures such that the flow of blood may pass through the blood vessel unencumbered.

### EXAMPLE IMPLANTATION OF FLOW MODULATING DEVICES

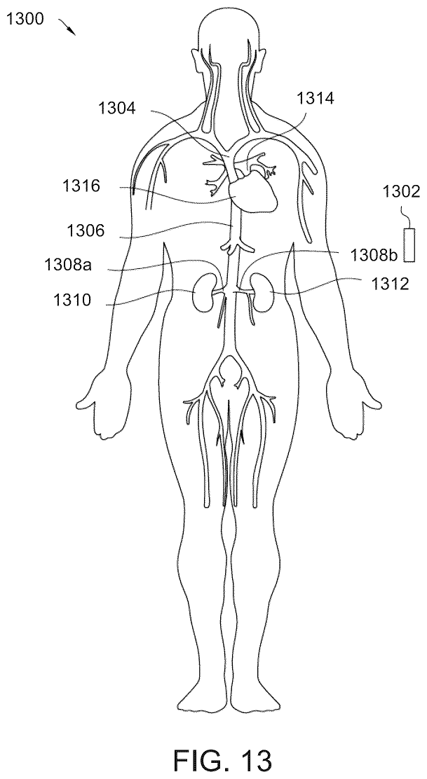

FIG. 13 illustrates a schematic representation of portions of a subject 1300. The flow modulating devices described herein of FIGS. 1A-11B (represented in FIG. 13 by device 1302) may be introduced (e.g., implanted) in vasculature of the body. In general, the device 1302 may represent any of the flow modulating devices described herein (e.g., device 100, device 200, device 300 device 400, device 500, device 600, device 700, device 750, device 800, device 900, system 1000, etc.) and may include the same or similar functionality and/or structures. In some examples, the device 1302 may be implanted in or near to a portion of the Superior Vena Cava (SVC) 1304. In some examples, the device 1302 may be implanted in or near to a portion of the Inferior Vena Cava (IVC) 1306. The subject 1300 is illustrated with a representation of a portion of the vasculature system to generally illustrate the SVC 1304 and the IVC 1306 within the subject 1300. However, it is to be understood that no dimensions or relative sizes of components may be inferred from the relative sizes and dimensions of elements in the figures.

The subject 1300 includes a number of vessels and organs that may circulate blood throughout the body. For example, renal veins 1308a and 1308b drain blood from respective right kidney 1310 and left kidney 1312. Renal veins 1308a and 1308b connect to the IVC 1306. Blood from the aorta 1314 flows to the IVC 1306. Blood travels from the aorta 1314 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 1306 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

Heart failure patients can have multiple comorbidities that cause excessive congestion or accumulation of blood volume in the splanchnic venous circulation. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized. For example, because blood flow from the splanchnic venous circulation is directed through hepatic veins and into the IVC 1306, devices (as described herein) may be placed into the IVC 1306 to limit blood flow to allow the splanchnic venous circulation to expand with increased blood volume. This may also allow the body to accumulate blood volume in the splanchnic venous circulation, which can maximize the downstream drop of pressure relative to upstream increase of pressure. Similarly, devices (as described herein) may be placed into the SVC 1308 to limit blood flow to allow the reservoir to expand with increased blood volume. Furthermore, the flow modulating devices described herein may be placed in either the IVC 1306 and/or SVC 1308 to alleviate pressure in the right side of the atrium of the heart 1316 and/or regulate renal venous pressure and kidney function. Another example positioning of a flow modulating device may be in the IVC below the renal veins. This positioning may have a similar effect as the SVC location, as it may allow the flow modulating device to maintain renal venous pressure, which can correlate with sustained renal function and diuresis.

In some examples, the flow modulating device 1302 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 1302 may be used as a method of treatment to regulate pressure in the right atrium of the heart. Further, the flow modulating device 1302 may be used as a method of treatment to improve function of the kidneys in patients having reduced kidney function due to pressure in the venous system.

For example, any of the implantable devices and/or systems described herein may be configured to modulate a volume of blood flowing from a superior vena cava into a right atrium to decrease right atrial pressure.

Further for example, any of the implantable devices and/or systems described herein may be used to perform a method including restricting blood flow within a blood vessel.

Still further for example, any of the implantable devices and/or systems described herein may be used to perform a method of treatment for a subject having one or both of: congestive heart failure or chronic kidney disease. The method may include restricting blood flow within the blood vessel.

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve-based system (e.g., with a manifold), hydraulics, pneumatics, optical actuators, thermal actuators, and/or other actuator using electrical or inductive power.

In some examples, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include a pump fluidly connected to a reservoir; a chamber having a first portion and a second portion; a manifold fluidly connected to the pump, the reservoir, and the chamber; and a piston coupled to a control element of a flow modulating device. The manifold may include at least one port that fluidly connects the reservoir to the first portion of the chamber. The piston can move between a restricted blood flow position and an unrestricted blood flow position within the chamber or any position therebetween for intermediate blood flow restriction positions. For example, the piston may move to the restricted blood flow position when a fluid flows from (or is pumped from) the reservoir through the manifold into the first portion of the chamber. The piston can return to the unrestricted blood flow position when the fluid is evacuated from the first portion of the chamber. In some embodiments, the manifold is fluidly connected to a second portion of the chamber through a second port. In such embodiments, the piston can move to the unrestricted blood flow position when the fluid enters the second port from the reservoir through the manifold, thereby causing the valve of the flow modulating device to move to the unrestricted blood flow state. In some embodiments, the fluid is evacuated from the second portion of the chamber through the second port when the piston is in the restricted blood flow position. In some implementations, the at least one port further fluidly connects the first portion of the chamber to the pump through the manifold. For example, the at least first port is fluidly connected to the pump through the manifold to evacuate the fluid from the first portion of the chamber thereby moving the piston to the unrestricted blood flow position. In some examples, the piston is a spring-based piston. For example, the spring-based piston can automatically return to the unrestricted blood flow position when the fluid is evacuated from the first portion of the chamber.

In any of the embodiments described herein, an active mechanism may include an actuator (e.g., a linear actuator) coupled to a control element of the flow management device. The linear actuator tensions the control element to position the valve of the flow management device in a restricted blood flow state. Alternatively, the linear actuator releases tension in the control element to position a valve, a membrane, or other material in an unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

In any of the embodiments described herein, the linear actuator is an electromechanical linear actuator having a first magnet that, when caused to rotate by another magnet or actuator, causes a nut to rotate on a lead screw, the nut being coupled to the control clement. A second magnet in a control device may cause rotation of the first magnet, for example by changing its magnetic field pole direction. In some embodiments, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the valve to an unrestricted blood flow state.

In any of the embodiments described herein, the linear actuator is a thermal linear actuator having a piston coupled to the control element. For example, decreasing a temperature of a thermal sensitive fluid (e.g., via a heat source, changes in body temperature, etc.) causes the piston to compress the fluid to tension the control element to move the valve into the restricted blood flow state. Alternatively, increasing the temperature of the thermal sensitive fluid causes the piston to decompress the fluid to release tension in the control element to move the valve to the unrestricted blood flow state.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, etc.), anatomy responses (e.g., changes in vessel inner diameter, intra-vessel pressure, etc.), blood movement, or the like.

Any of the implantable or flow modulating devices described herein may be coated with a polymer (e.g., silicones, poly (urethanes), poly (acrylates), or copolymers such as poly (ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly (ethylene terephthalate), while the nonwoven fabrics may be made from expanded poly (tetrafluoroethylene). Some textiles may also or alternatively include silk or silk-based materials.

Further, any of the pull wires, sutures, frames/stents, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

The flow modulating devices described herein may be part of (or installed within) a stent. The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents described herein may be configured to house all or a portion of the flow modulating devices described herein. Such stents may include an assembly with strut members interconnected by joints that form a series of linked mechanisms that result in a hollow tube-shaped element. The stents may be positioned and/or repositioned within a blood vessel to introduce or remove flow modulating devices or device members including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

The stents described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Any of the embodiments described herein may be configured to alleviate blood stasis within a potential stasis zone. For example, any of the flow modulating devices described herein may include one or more stasis reduction solutions described herein. While particular stasis reducing features may not be described for the embodiments of FIGS. 2A-9D for brevity, any or all of the embodiments of FIGS. 2A-9D may incorporate one or more of the stasis reducing features described in FIGS. IC-1J to reduce or eliminate blood and particulate stasis and/or to eliminate stasis zones. The stasis reducing features may include, but are not limited to defined pockets, conduits, gutters, grooves, paths, perforations, apertures, flaps, slots, indentations, or the like. Such features may be punctured, cut, or manufactured for placement within portions of a respective frame and/or within portions of a respective membrane, mesh, or balloon. Further, any combination of shapes and/or any combination of sizes of the stasis reducing features may be contemplated to reduce stasis when used with one or more of the flow modulating devices described herein. In addition, any of the stasis reducing features described herein may be combined with any number of other stasis reducing features to reduce stasis within a particular flow modulating device and/or within vessels around or substantially adjacent to the particular flow modulating device.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1. An implantable device for modulating blood flow through a blood vessel, the implantable device comprising: an expandable frame comprising a proximal end and a distal end and a longitudinal axis extending therethrough; and a membrane comprising an inflow end and an outflow end, wherein the inflow end is at least partially installed within the distal end of the expandable frame, and the outflow end is coupled to a plurality of elongate support members arranged radially around an outer surface of the membrane and extending substantially parallel to the longitudinal axis, and wherein the membrane is configured to radially collapse at the outflow end and toward a central axis of the expandable frame, or radially expand away from the central axis of the expandable frame, in response to an actuation of a control wire coupled to a portion of the membrane or at least one of the plurality of elongate support members, the central axis of the expandable frame being substantially parallel to the longitudinal axis.

Example 2. The implantable device of any of the preceding examples, but particularly example 1, wherein the plurality of elongate support members is flexible to allow the membrane to bend radially toward the central axis of the expandable frame at the outflow end when the control wire is actuated.

Example 3. The implantable device of any of the preceding examples, but particularly example 1, wherein actuating the control wire results in configuring the membrane in an unrestricted blood flow state or a restricted blood flow state, wherein: the unrestricted blood flow state corresponds to the membrane radially expand away from the central axis of the expandable frame to allow blood flow through the blood vessel; and the restricted blood flow state corresponds to the membrane radially collapsing toward the central axis of the expandable frame to reduce blood flow through the blood vessel.

Example 4. The implantable device of any of the preceding examples, but particularly example 1, wherein the membrane is adjustable to a plurality of positions between expanded and collapsed, the plurality of positions including at least: an expanded position configured to allow the blood flow through the blood vessel; a partially expanded position configured to partially occlude the blood vessel; and a collapsed position configured to block the outflow end to occlude the blood vessel.

Example 5. The implantable device of any of the preceding examples, but particularly example 4, further comprising: a plurality of eyelets, wherein each respective eyelet of the plurality of eyelets is coupled to a distal end of each corresponding elongate support member in the plurality of elongate support members, each eyelet being configured to receive a portion of the control wire threaded therethrough such that the actuation of the control wire reversibly cinches the membrane by bringing the plurality of eyelets together at the outflow end to occlude or partially occlude the blood vessel.

Example 6. The implantable device of any of the preceding examples, but particularly example 5, wherein the cinching may be performed to: fully collapse the outflow end of the membrane resulting in occlusion of the blood vessel; or partially collapse the outflow end of the membrane resulting in a partial occlusion of the blood vessel.

Example 7. The implantable device of any of the preceding examples, but particularly example 5, wherein each respective eyelet in the plurality of eyelets is configured to attach to the distal end of a respective elongate support member in the plurality of elongate support members, and extends beyond the outflow end of the membrane.

Example 8. The implantable device of any of the preceding examples, but particularly example 5, wherein each of the plurality of eyelets is substantially annular, and wherein an aperture of each respective eyelet in the plurality of eyelets is arranged to receive the control wire when threaded therethrough such that when the control wire is actuated, the plurality of eyelets move radially toward the central axis of the expandable frame.

Example 9. The implantable device of any of the preceding examples, but particularly example 1, wherein the membrane is: substantially tubular-shaped with a substantially circular cross section; and adjustable to form a cinched portion at the outflow end, the cinching resulting in reversibly reducing or closing the circular cross section at the outflow end.

Example 10. The implantable device of any of the preceding examples, but particularly example 1, wherein the blood vessel comprises a superior vena cava or an inferior vena cava.

Example 11. The implantable device of any of the preceding examples, but particularly example 1, further comprising a power source coupled to the control wire, the power source comprising a battery or a wall outlet.

Example 12. The implantable device of any of the preceding examples, but particularly example 1, further comprising: an actuation device for actuating the control wire of the implantable device, the actuation device comprising: an actuator coupled to the control wire of the implantable device, and a first magnet configured to induce rotation of the actuator; and a control device communicatively coupled to the actuator, wherein the control device comprises a second magnet configured to generate a changing magnetic field pole direction to cause rotation of the first magnet.

Example 13. The implantable device of any of the preceding examples, but particularly example 12, wherein: the actuation device is configured to send a first signal to the control wire to activate application of tension to the control wire; and the actuation device is configured to send a second signal to the control wire to activate release of the tension from the control wire.

Example 14. The implantable device of any of the preceding examples, but particularly example 12, wherein the actuation device is further configured to: cause rotation of the second magnet in a first direction to induce rotation of the first magnet, thereby causing the actuator to tension the control wire to cause the membrane to radially collapse inward at the outflow end; and cause rotation of the second magnet in a second direction to induce rotation of the first magnet, thereby causing the actuator to release tension in the control wire to cause the membrane to radially open at the outflow end.

Example 15. The implantable device of any of the preceding examples, but particularly example 12, wherein the actuator is a magnetically driven actuator.

Example 16. The implantable device of any of the preceding examples, but particularly example 12, wherein the control device is implanted.

Example 17. The implantable device of any of the preceding examples, but particularly example 12, wherein the control device is implanted subcutaneously.

Example 18. The implantable device of any of the preceding examples, but particularly example 12, wherein the control device is disposed external to a body of a user associated with the implantable device.

Example 19. The implantable device of any of the preceding examples, but particularly example 12, wherein the first magnet is a permanent magnet, and the second magnet is a permanent magnet.

Example 20. The implantable device of any of the preceding examples, but particularly example 12, wherein the first magnet is a permanent magnet, and the second magnet is an electromagnet.

Example 21. The implantable device of any of the preceding examples, but particularly example 1, wherein the implantable device is configured to modulate a volume of blood flowing from a superior vena cava into a right atrium to decrease right atrial pressure.

Example 22. The implantable device of any of the preceding examples, but particularly example 1, wherein the membrane includes a plurality of perforations in a substantially helical arrangement about a central axis and extending at equal intervals from one another along the outer surface of the membrane, the central axis being substantially parallel to the longitudinal axis.

Example 23. The implantable device of any of the preceding examples, but particularly example 22, wherein the plurality of perforations allow blood to flow from a plurality of stasis zones to cause a reduction in blood stasis in or around the membrane.

Example 24. The implantable device of any of the preceding examples, but particularly example 1, wherein the membrane includes a plurality of perforations substantially adjacent to the outflow end and extending around at least a portion of the outer surface of the membrane.

Example 25. The implantable device of any of the preceding examples, but particularly example 24, wherein the plurality of perforations comprises slots in the membrane.

Example 26. The implantable device of any of the preceding examples, but particularly example 24, wherein the plurality of perforations comprises arc-shaped flaps in the membrane.

Example 27. The implantable device of any of the preceding examples, but particularly example 24, wherein the plurality of perforations comprises apertures in the membrane.

Example 28. The implantable device of any of the preceding examples, but particularly example 24, wherein the plurality of perforations allow blood to flow from a plurality of stasis zones to cause a reduction in blood stasis in or around the membrane.

Example 29. The implantable device of any of the preceding examples, but particularly example 1, further comprising a plurality of grooves along an inner surface of the expandable frame.

Example 30. The implantable device of any of the preceding examples, but particularly example 29, wherein the plurality of grooves enable blood to flow along portions of the expandable frame from the inflow end to the outflow end to reduce blood stasis around the implantable device.

Example 31. The implantable device of any of the preceding examples, but particularly example 29, wherein the plurality of grooves is shaped in a spiral or helix along the outer surface of at least a portion of the membrane.

Example 32. A method of treatment for reducing cardiac blood flow at a target site in a blood vessel of a heart of a subject, the method comprising: introducing a device in the blood vessel, the device comprising: an expandable frame comprising a proximal end and a distal end and a longitudinal axis extending therethrough; and a membrane comprising an inflow end and an outflow end, wherein the inflow end is at least partially installed within the distal end of the expandable frame, and the outflow end is coupled to a plurality of elongate support members arranged radially around an outer surface of the membrane and extending substantially parallel to the longitudinal axis, and wherein the membrane is configured to radially collapse at the outflow end and toward a central axis of the expandable frame, or radially expand away from the central axis of the expandable frame, in response to an actuation of a control wire coupled to a portion of the membrane or at least one of the plurality of elongate support members; and actuating the device to modulate a flow of blood within the blood vessel.

Example 33. The method of any of the preceding examples, but particularly example 32, further comprising: de-actuating the device to maintain or regain the flow of blood within the blood vessel.

Example 34. The method of any of the preceding examples, but particularly example 32, wherein the target site includes a portion of a superior vena cava (SVC) of a subject in which the device is implanted, or a portion of an inferior vena cava (IVC) of the subject.

Example 35. The method of any of the preceding examples, but particularly example 32, wherein actuating the device causes a partial occlusion of blood in the blood vessel.

Example 36. The method of any of the preceding examples, but particularly example 32, wherein the central axis of the expandable frame is substantially parallel to the longitudinal axis.

Example 37. The method of any of the preceding examples, but particularly example 32, wherein the blood vessel is a superior vena cava and the device is configured to be implanted in a portion of the superior vena cava of a subject having chronic kidney disease and chronic heart failure; and the method further comprises modulating a volume of blood flowing from the blood vessel into a right atrium to decrease right atrial pressure.

Example 38. A device for modulating blood flow through a blood vessel, the device comprising: a housing comprising a proximal end and a distal end and a longitudinal axis extending therethrough; and a plurality of elongate projections arranged radially around an outer surface of the distal end of the housing and extending substantially parallel to the longitudinal axis, each elongate projection in the plurality of elongate projections having a first end portion coupled to the distal end of the housing and a second end portion configured to form a portion of a nozzle, and a control element coupled to a portion of the housing, wherein the plurality of elongate projections is configured to radially collapse at the respective second end portions and toward a central axis of the housing, or radially expand away at the respective second end portions from the central axis of the housing, in response to actuation of the control element,

Example 39. The device of any of the preceding examples, but particularly example 38, wherein the central axis of the housing is substantially parallel to the longitudinal axis.

Example 40. The device of any of the preceding examples, but particularly example 38, wherein the nozzle is configured to: fully restrict a flow of blood resulting in occlusion of the blood vessel; and partially restrict the flow of blood resulting in a partial occlusion of the blood vessel.

Example 41. The device of any of the preceding examples, but particularly example 38, wherein the plurality of elongate projections are coupled to a housing base and are configured to radially expand or radially collapse in response to actuation of the control element, and wherein: actuating the control element results in twisting the housing base in a first direction to radially expand the plurality of elongate projections to allow blood flow through the blood vessel.

Example 42. The device of any of the preceding examples, but particularly example 41, further comprising re-actuating the control element, the re-actuating resulting in twisting the housing base in a second direction to radially collapse the plurality of elongate projections to reduce blood flow through the blood vessel.

Example 43. The device of any of the preceding examples, but particularly example 38, wherein the plurality of elongate projections is wedge-shaped.

Example 44. The device of any of the preceding examples, but particularly example 38, wherein the plurality of elongate projections is triangle shaped.

Example 45. The device of any of the preceding examples, but particularly example 38, wherein the plurality of elongate projections is rectangular-shaped.

Example 46. The device of any of the preceding examples, but particularly example 38, wherein the plurality of elongate projections is polygon shaped.

Example 47. An implantable device for modulating blood flow through a blood vessel, the implantable device comprising: an expandable frame comprising a proximal end and a distal end and a longitudinal axis extending therethrough; a first membrane portion configured to attach to a first internal portion of the expandable frame; and a second membrane portion configured to attach to a second internal portion of the expandable frame, wherein the first internal portion is opposite the second internal portion; and a motor coupled to the first membrane portion and the second membrane portion and configured to reversibly expand one or both of the first membrane portion or the second membrane portion to reduce blood flow through the blood vessel.

Example 48. The implantable device of any of the preceding examples, but particularly example 47, wherein the motor is a linear actuator coupled to the first membrane portion and the second membrane portion.

Example 49. The implantable device of any of the preceding examples, but particularly example 48, wherein the expandable frame further defines a lumen therethrough from the proximal end to the distal end and wherein the first membrane portion or the second membrane portion are configured to expand toward a central axis of the expandable frame to cause a narrowing of the lumen.

Example 50. The implantable device of any of the preceding examples, but particularly example 48, wherein the first membrane portion and the second membrane portion are elastic and configured to expand and contract to regulate blood flow through the blood vessel, wherein: expanding the first membrane portion or the second membrane portion within the expandable frame results in partially occluding the blood vessel; and expanding both the first membrane portion within the expandable frame and the second membrane portion within the expandable frame results in fully occluding the blood vessel.

Example 51. The implantable device of any of the preceding examples, but particularly example 50, wherein the first membrane portion and the second membrane portion are dome shaped.

Example 52. The implantable device of any of the preceding examples, but particularly example 50, wherein: contracting the first membrane portion within the expandable frame and contracting the second membrane portion within the expandable frame results in restoring blood flow through the blood vessel; and contracting the first membrane portion or contracting the second membrane portion within the expandable frame results in partially restoring blood flow through the blood vessel.

Example 53. The implantable device of any of the preceding examples, but particularly example 52, wherein: the expanding of the first membrane portion or the second membrane portion is performed in response to actuation of a control wire using the motor, the control wire being coupled to the first membrane portion and the second membrane portion; and the contracting of the first membrane portion or the second membrane portion is performed in response to actuation of the control wire using the motor.

Example 54. The implantable device of any of the preceding examples, but particularly example 47, further comprising: a sensor configured to detect a pressure in the blood vessel; and a microprocessor electrically coupled to the sensor, wherein the motor is electrically coupled to a linear actuator, the microprocessor, and the sensor.

Example 55. The implantable device of any of the preceding examples, but particularly example 54, wherein the sensor comprises one of: a strain gauge, a piezoelectric sensor, a capacitance sensor, or a vacuum pressure sensor.

Example 56. The implantable device of any of the preceding examples, but particularly example 54, wherein the microprocessor is configured to: receive a signal from the sensor, the signal being indicative of a pressure in the blood vessel; and output a control signal to the linear actuator to either expand the first membrane portion or contract the first membrane portion based on the sensed pressure in the blood vessel; and output a control signal to the linear actuator to either expand the second membrane portion or contract the second membrane portion based on the sensed pressure in the blood vessel.

Example 57. The implantable device of any of the preceding examples, but particularly example 47, wherein the blood vessel comprises a superior vena cava or an inferior vena cava.

Example 58. The implantable device of any of the preceding examples, but particularly example 47, wherein the expandable frame has a pro-endothelialization coating, such that the expandable frame is configured to be at least partially incorporated into an inner wall of the blood vessel.

Example 59. The implantable device of any of the preceding examples, but particularly example 47, wherein the expandable frame comprises a bare metal stent, such that the expandable frame is configured to be at least partially incorporated into an inner wall of the blood vessel.

Example 60. An implantable device for modulating blood flow through a blood vessel, the implantable device comprising: an expandable frame defining a lumen therethrough, wherein the expandable frame is transitionable between a constricted configuration and an unconstricted configuration; a control element at least partially circumferentially disposed about the expandable frame; and a mechanical actuator configured to tension the control clement to transition the expandable frame to the constricted configuration and to release tension in the control element to transition the expandable frame to the unconstricted configuration.

Example 61. The implantable device of any of the preceding examples, but particularly example 60, wherein the expandable frame comprises a first end portion, an intermediate portion, and a second end portion.

Example 62. The implantable device of any of the preceding examples, but particularly example 61, wherein the control element is at least partially circumferentially disposed about the intermediate portion of the expandable frame.

Example 63. The implantable device of any of the preceding examples, but particularly example 61, wherein the mechanical actuator is configured to tension the control clement to transition the intermediate portion of the expandable frame to the constricted configuration and to release tension in the control element to transition the intermediate portion of the expandable frame to the unconstricted configuration.

Example 64. The implantable device of any of the preceding examples, but particularly example 60, wherein the control element comprises a wire.

Example 65. The implantable device of any of the preceding examples, but particularly example 60, wherein: the mechanical actuator comprises a catheter; and the control clement is axially translatable through a lumen of the catheter to tension or release tension in the control element.

Example 66. The implantable device of any of the preceding examples, but particularly example 60, wherein the mechanical actuator comprises a linear actuator.

Example 67. The implantable device of any of the preceding examples, but particularly example 66, wherein the linear actuator is one of: a pneumatic linear actuator, an electromechanical linear actuator, or a hydraulic linear actuator.

Example 68. The implantable device of any of the preceding examples, but particularly example 60, further comprising: a sensor configured to detect a pressure in the blood vessel; a microprocessor electrically coupled to the sensor; and a power source electrically coupled to the mechanical actuator, the microprocessor, and the sensor.

Example 69. The implantable device of any of the preceding examples, but particularly example 68, wherein the sensor comprises one of: a strain gauge, a piezoelectric sensor, a capacitance sensor, or a vacuum pressure sensor.

Example 70. The implantable device of any of the preceding examples, but particularly example 68, wherein the power source comprises an induction coil.

Example 71. The implantable device of any of the preceding examples, but particularly example 68, wherein the microprocessor is configured to: receive a signal from the sensor, the signal being indicative of a pressure in the blood vessel; and output a control signal to tension the control element or release tension in the control element based on the sensed pressure in the blood vessel.

Example 72. The implantable device of any of the preceding examples, but particularly example 60, wherein the expandable frame comprises a self-expanding stent.

Example 73. The implantable device of any of the preceding examples, but particularly example 72, wherein the self-expanding stent is coated with a membrane.

Example 74. The implantable device of any of the preceding examples, but particularly example 72, wherein an outer diameter of the expandable frame is configured to substantially form a seal with an inner wall of the blood vessel.

Example 75. The implantable device of any of the preceding examples, but particularly example 60, wherein the constricted configuration of the expandable frame is configured to restrict blood flow through the lumen of the expandable frame.

Example 76. The implantable device of any of the preceding examples, but particularly example 60, wherein the blood vessel comprises a superior vena cava or an inferior vena cava.

Example 77. The implantable device of any of the preceding examples, but particularly example 60, wherein the expandable frame has a pro-endothelialization coating, such that the expandable frame is configured to be at least partially incorporated into an inner wall of the blood vessel.

Example 78. The implantable device of any of the preceding examples, but particularly example 60, wherein the expandable frame comprises a bare metal stent, such that the expandable frame is configured to be at least partially incorporated into an inner wall of the blood vessel.

Example 79. An implantable device for modulating blood flow through a blood vessel, the implantable device comprising: a frame defining a lumen therethrough, wherein the frame is transitionable between a constricted configuration and an unconstricted configuration; a substantially tubular-shaped membrane provided within a partial length of the lumen, the membrane having a first end portion, an intermediate portion, and a second end portion; and a control element disposed within a portion of the frame, the control element including a proximal end and a distal end, the proximal end being coupled to the membrane and the distal end being coupled to a mechanical actuator configured to actuate the control element to cause the frame to transition between the constricted configuration and the unconstricted configuration.

Example 80. The implantable device of any of the preceding examples, but particularly example 79, wherein the constricted configuration comprises narrowing the intermediate portion of the membrane while the first end portion and the second end portion of the membrane are translated toward the intermediate portion.

Example 81. The implantable device of any of the preceding examples, but particularly example 79, wherein the unconstricted configuration comprises expanding the intermediate portion of the membrane while the first end portion and the second end portion of the membrane are translated away from the intermediate portion.

Example 82. The implantable device of any of the preceding examples, but particularly example 79, further comprising: wherein the intermediate portion is further configured to expand while the first end portion and the second end portion increase in length while being translated away from the intermediate portion, responsive to the frame being transitioned to the constricted configuration, wherein the first end portion remains coupled to the intermediate portion and the intermediate portion remains coupled to the second end portion.

Example 83. The implantable device of any of the preceding examples, but particularly example 79, wherein the constricted configuration of the frame is configured to restrict blood flow through the membrane.

Example 84. The implantable device of any of the preceding examples, but particularly example 83, wherein the constricted configuration causes a reduction in the blood flow through the membrane by at least about 50 percent.

Example 85. The implantable device of any of the preceding examples, but particularly example 83, wherein the constricted configuration causes a reduction in the blood flow through the membrane by about 50 percent to about 75 percent.

Example 86. The implantable device of any of the preceding examples, but particularly example 79, wherein the intermediate portion is narrowed to form a dumbbell shape within the membrane, the dumbbell shape configured to reduce an amount of blood allowed to flow through the blood vessel.

Example 87. The implantable device of any of the preceding examples, but particularly example 79, wherein the control element comprises a wire or a suture.

Example 88. The implantable device of any of the preceding examples, but particularly example 79, wherein the mechanical actuator comprises a catheter, and wherein the control element is axially translatable within a lumen of the catheter to tension in the control element to place the implantable device in the constricted configuration and release tension in the control element to place the implantable device in the unconstricted configuration.

Example 89. The implantable device of any of the preceding examples, but particularly example 79, wherein the mechanical actuator comprises a linear actuator.

Example 90. The implantable device of any of the preceding examples, but particularly example 89, wherein the linear actuator is one of: a pneumatic linear actuator, an electromechanical linear actuator, or a hydraulic linear actuator.

Example 91. The implantable device of any of the preceding examples, but particularly example 79, wherein the blood vessel comprises a superior vena cava or an inferior vena cava.

Example 92. An implantable device for modulating blood flow through a blood vessel, the implantable device comprising: an expandable frame defining a lumen therethrough, wherein the expandable frame is transitionable between a constricted configuration and an unconstricted configuration; a first control element at least partially circumferentially disposed about the expandable frame; a second control element at least partially circumferentially disposed about the expandable frame; and a mechanical actuator configured to tension the first control element and the second control element to transition the expandable frame to the constricted configuration and to release tension in the first control element or the second control element to transition the expandable frame to the unconstricted configuration.

Example 93. The implantable device of any of the preceding examples, but particularly example 92, wherein the expandable frame comprises a first end portion, an intermediate portion, and a second end portion.

Example 94. The implantable device of any of the preceding examples, but particularly example 93, wherein the first control element is at least partially circumferentially disposed about a first location on the intermediate portion of the expandable frame.

Example 95. The implantable device of any of the preceding examples, but particularly example 94, wherein the second control element is at least partially circumferentially disposed about a second location on the intermediate portion of the expandable frame.

Example 96. The implantable device of any of the preceding examples, but particularly example 95, wherein the mechanical actuator is configured to tension the first control element to transition the first location on the intermediate portion of the expandable frame to the constricted configuration and to release tension in the first control element to transition the first location on the intermediate portion of the expandable frame to the unconstricted configuration.

Example 97. The implantable device of any of the preceding examples, but particularly example 95, wherein the mechanical actuator is configured to tension the second control element to transition the second location on the intermediate portion of the expandable frame to the constricted configuration and to release tension in the second control element to transition the second location on the intermediate portion of the expandable frame to the unconstricted configuration.

Example 98. The implantable device of any of the preceding examples, but particularly example 92, wherein tensioning both the first control element and the second control element results in occluding the blood vessel.

Example 99. The implantable device of any of the preceding examples, but particularly example 92, wherein tensioning the first control element or the second control clement results in partially occluding the blood vessel.

Example 100. The implantable device of any of the preceding examples, but particularly example 92, wherein the first control element comprises a substantially annular wire and the second control element comprises a substantially annular wire.

Example 101. The implantable device of any of the preceding examples, but particularly example 100, wherein at least one of the first control element or the second control clement are formed of a shape memory alloy.

Example 102. The implantable device of any of the preceding examples, but particularly example 92, wherein the mechanical actuator comprises a linear actuator.

Example 103. The implantable device of any of the preceding examples, but particularly example 102, wherein the linear actuator is one of: a pneumatic linear actuator, an electromechanical linear actuator, or a hydraulic linear actuator.

Example 104. The implantable device of any of the preceding examples, but particularly example 92, further comprising: a sensor configured to detect a pressure in the blood vessel; a microprocessor electrically coupled to the sensor; and a power source electrically coupled to the mechanical actuator, the microprocessor, and the sensor.

Example 105. The implantable device of any of the preceding examples, but particularly example 104, wherein the sensor comprises one of: a strain gauge, a piezoelectric sensor, a capacitance sensor, or a vacuum pressure sensor.

Example 106. The implantable device of any of the preceding examples, but particularly example 104, wherein the power source comprises an induction coil.

Example 107. The implantable device of any of the preceding examples, but particularly example 104, wherein the microprocessor is configured to: receive a signal from the sensor, the signal being indicative of a pressure in the blood vessel; and output a control signal to tension the first control element or the second control element or release tension in the first control element or the second control element, based on the sensed pressure in the blood vessel.

Example 108. The implantable device of any of the preceding examples, but particularly example 92, wherein the expandable frame comprises a self-expanding stent.

Example 109. The implantable device of any of the preceding examples, but particularly example 108, wherein the self-expanding stent is coated with a membrane.

Example 110. The implantable device of any of the preceding examples, but particularly example 108, wherein an outer diameter of the expandable frame is configured to substantially form a seal with an inner wall of the blood vessel.

Example 111. The implantable device of any of the preceding examples, but particularly example 92, wherein the blood vessel comprises a superior vena cava or an inferior vena cava.

Example 112. The implantable device of any of the preceding examples, but particularly example 92, wherein the expandable frame comprises a bare metal stent, such that the expandable frame is configured to be at least partially incorporated into an inner wall of the blood vessel.

Example 113. An implantable device for modulating blood flow through a blood vessel, the implantable device comprising: an expandable frame defining a lumen therethrough, wherein the expandable frame is transitionable between a constricted configuration and an unconstricted configuration; a first insert configured to at least partially attach to a first interior wall of the expandable frame; a second insert configured to at least partially attach to a second interior wall of the expandable frame, the first interior wall being opposite the second interior wall; a mechanical actuator configured to tension the first insert or the second insert to transition the expandable frame to the constricted configuration and to release tension in the first insert or the second insert to transition the expandable frame to the unconstricted configuration.

Example 114. The implantable device of any of the preceding examples, but particularly example 113, wherein: the first insert is a first wire within the expandable frame, the first wire being formed in an S-curve shape; and the second insert is a second wire within the expandable frame, the second wire being formed in an inverted S-curve shape.

Example 115. The implantable device of any of the preceding examples, but particularly example 114, wherein the mechanical actuator is configured to: slide the first insert to transition along the first interior wall from a predefined position to the constricted configuration, wherein sliding the first insert results in aligning an intermediate portion of the first wire with an intermediate portion of the second wire, the alignment providing at least a partial occlusion of the blood vessel; and slide the first insert to transition along the first interior wall to the unconstricted configuration by sliding the first insert to the predefined position to align the intermediate portion of the first wire with an end portion of the second wire.

Example 116. The implantable device of any of the preceding examples, but particularly example 115, wherein the predefined configuration is the unconstricted configuration.

Example 117. The implantable device of any of the preceding examples, but particularly example 116, wherein the blood vessel comprises a superior vena cava or an inferior vena cava.

Example 118. The implantable device of any of the preceding examples, but particularly example 113, further comprising: a sensor configured to detect a pressure in the blood vessel; a microprocessor electrically coupled to the sensor; and a power source electrically coupled to the mechanical actuator, the microprocessor, and the sensor.

Example 119. The implantable device of any of the preceding examples, but particularly example 118, wherein the sensor comprises one of: a strain gauge, a piezoelectric sensor, a capacitance sensor, or a vacuum pressure sensor.

Example 120. The implantable device of any of the preceding examples, but particularly example 118, wherein the power source comprises an induction coil.

Example 121. The implantable device of any of the preceding examples, but particularly example 118, wherein the microprocessor is configured to: receive a signal from the sensor, the signal being indicative of the pressure in the blood vessel; and output a control signal to the mechanical actuator to slide the first insert from a predefined position to the constricted configuration or return the first insert to the predefined position.

Example 122. A device for modulating blood flow through a blood vessel, the device being configured to be at least partially circumferentially disposed about an outer wall of the blood vessel, comprising: a ring having a control element circumferentially disposed in the ring, wherein the control element has a first looped end and a second free end that passes through the first looped end, and wherein the control element is configured to translate relative to an inner diameter of the ring; and a mechanical actuator configured to be coupled to the second free end of the control element, wherein the mechanical actuator is configured to: apply tension to the second free end to reduce a diameter of the control element disposed in the ring; and reduce tension on the second free end to maintain or increase the diameter of the control clement disposed in the ring.

Example 123. The device of any of the preceding examples, but particularly example 122, wherein the application of the tension to the second free end results in reducing a diameter of the blood vessel.

Example 124. The device of any of the preceding examples, but particularly example 122, wherein the reduction of the tension on the second free end results in maintaining or increasing the diameter of the blood vessel.

Example 125. The device of any of the preceding examples, but particularly example 122, wherein the ring is a polymeric ring.

Example 126. The device of any of the preceding examples, but particularly example 125, wherein the polymeric ring comprises one or more of: silicone, polyurethane, polyacrylate, or polyethylene vinyl acetate.

Example 127. The device of any of the preceding examples, but particularly example 122, wherein the ring comprises a textile.

Example 128. The device of any of the preceding examples, but particularly example 122, wherein the ring comprises tissue.

Example 129. The device of any of the preceding examples, but particularly example 122, wherein the ring is a substantially circular spring.

Example 130. The device of any of the preceding examples, but particularly example 122, wherein the control element comprises a wire or a suture.

Example 131. The device of any of the preceding examples, but particularly example 122, wherein the mechanical actuator comprises a catheter, and wherein the control element is axially translatable within a lumen of the catheter to tension or release tension in the control element.

Example 132. The device of any of the preceding examples, but particularly example 122, wherein the mechanical actuator comprises a linear actuator.

Example 133. The device of any of the preceding examples, but particularly example 132, wherein the linear actuator is one of: a pneumatic linear actuator, an electromechanical linear actuator, or a hydraulic linear actuator.

Example 134. The device of any of the preceding examples, but particularly example 122, further comprising: a sensor configured to detect a pressure in the blood vessel; a microprocessor electrically coupled to the sensor; and a power source electrically coupled to the mechanical actuator, the microprocessor, and the sensor.

Example 135. The device of any of the preceding examples, but particularly example 134, wherein the sensor comprises one of: a strain gauge, a piezoelectric sensor, a capacitance sensor, or a vacuum pressure sensor.

Example 136. The device of any of the preceding examples, but particularly example 134, wherein the power source comprises an induction coil.

Example 137. The device of any of the preceding examples, but particularly example 134, wherein the microprocessor is configured to: receive a signal from the sensor, the signal being indicative of the pressure in the blood vessel; and output a control signal to the mechanical actuator to either tension the control element or release tension in the control element based on the sensed pressure in the blood vessel.

Example 138. The device of any of the preceding examples, but particularly example 122, wherein the tensioned control element is configured to restrict blood flow through the blood vessel.

Example 139. The device of any of the preceding examples, but particularly example 122, wherein the blood vessel comprises a superior vena cava or an inferior vena cava.

Example 140. An implantable device for modulating blood flow through a blood vessel, the implantable device comprising: a frame defining a lumen therethrough, wherein the frame is transitionable between a constricted configuration and an unconstricted configuration; a lever arm coupled to a portion of an inner surface of the frame and at least partially covered by a tissue portion; a control clement disposed between the frame and the lever arm and configured to move the lever arm from a first position to a second position; and a mechanical actuator configured to cause the control element to: transition the device from the unconstricted configuration to the constricted configuration by moving the lever arm from the first position to the second position; and transition the device from the constricted configuration to the unconstricted configuration by moving the lever arm from the second position to the first position.

Example 141. The implantable device of any of the preceding examples, but particularly example 140, wherein: the first position comprises the lever arm being parallel to a longitudinal axis defined by the frame; and the second position comprises the lever arm being at an acute angle from a perpendicular to the longitudinal axis defined by the frame, the second position causing the lever arm to engage the tissue portion and move the tissue portion toward a center axis of the lumen.

Example 142. The implantable device of any of the preceding examples, but particularly example 140, wherein the tissue portion is an elasticized tissue.

Example 143. The implantable device of any of the preceding examples, but particularly example 140, wherein the lever arm is formed of a shape memory alloy and is configured to elongate to raise the tissue portion when moving from the first position to the second position in response to receiving a first actuation signal from the mechanical actuator.

Example 144. The implantable device of any of the preceding examples, but particularly example 143, wherein the first actuation signal triggers the tissue portion to move to the constricted configuration.

Example 145. The implantable device of any of the preceding examples, but particularly example 140, wherein the lever arm is formed of a shape memory alloy and is configured to contract to lower the lever arm from the second position to the first position in response to receiving a second actuation signal from the mechanical actuator.

Example 146. The implantable device of any of the preceding examples, but particularly example 145, wherein the second actuation signal triggers the tissue portion to move to the unconstricted configuration.

Example 147. The implantable device of any of the preceding examples, but particularly example 140, wherein the frame is formed of one or more of: a polymer, a biomaterial, or a textile.

Example 148. The implantable device of any of the preceding examples, but particularly example 140, wherein the mechanical actuator is configured to tension the control clement to transition the lever arm to the constricted configuration and to release tension in the control element to transition the lever arm to the unconstricted configuration.

Example 149. The implantable device of any of the preceding examples, but particularly example 140, further comprising: a sensor configured to detect a pressure in the blood vessel; a microprocessor electrically coupled to the sensor; and a power source electrically coupled to the mechanical actuator, the microprocessor, and the sensor.

Example 150. The implantable device of any of the preceding examples, but particularly example 149, wherein the sensor comprises one of: a strain gauge, a piezoelectric sensor, a capacitance sensor, or a vacuum pressure sensor.

Example 151. The implantable device of any of the preceding examples, but particularly example 149, wherein the power source comprises an induction coil.

Example 152. The implantable device of any of the preceding examples, but particularly example 149, wherein the microprocessor is configured to: receive a signal from the sensor, the signal being indicative of the pressure in the blood vessel; and output a control signal to the mechanical actuator to either tension the control element or release tension in the control element based on the sensed pressure in the blood vessel.

Example 153. The implantable device of any of the preceding examples, but particularly example 152, wherein the tensioned control element is configured to raise the lever arm to restrict blood flow through the blood vessel.

Example 154. The implantable device of any of the preceding examples, but particularly example 140, wherein the blood vessel comprises a superior vena cava or an inferior vena cava.

Example 155. An implantable device for modulating blood flow through a blood vessel, the implantable device comprising: a first annular member comprising two or more first end anchors arranged circumferentially around the first annular member; a second annular member comprising two or more intermediate anchors arranged circumferentially around the second annular member, wherein the second annular member is transitionable between a constricted configuration and an unconstricted configuration; a third annular member comprising two or more second end anchors arranged circumferentially around the third annular member; a plurality of frame wires configured to: connect each of the two or more first end anchors to a respective intermediate anchor in the two or more intermediate anchors, and connect each of the two or more intermediate anchors to a respective end anchor in the two or more second end anchors; and a mechanical actuator configured to tension the plurality of frame wires to transition the second annular member to the constricted configuration and to release tension in the plurality of frame wires to transition the second annular member to the unconstricted configuration.

Example 156. The implantable device of any of the preceding examples, but particularly example 155, wherein the second annular member is a wire member formed of a shape memory polymer, the wire member being configured to connect the two or more intermediate anchors in sequence around the annular member.

Example 157. The implantable device of any of the preceding examples, but particularly example 156, wherein the mechanical actuator is further configured to tension the wire member to transition the second annular member to the constricted configuration and to release tension in the wire member to transition the second annular member to the unconstricted configuration.

Example 158. The implantable device of any of the preceding examples, but particularly example 155, wherein the plurality of frame wires is composed of a shape memory alloy to allow the two or more intermediate anchors to bend radially toward a central axis of the device when the plurality of frame wires are tensioned and to move radially away from the central axis of the device when the plurality of frame wires are released from tension.

Example 159. The implantable device of any of the preceding examples, but particularly example 155, wherein: tensioning the plurality of frame wires results in configuring the second annular member in a restricted blood flow state, and releasing tension in the plurality of wires results in configuring the second annular member in an unrestricted blood flow state.

Example 160. The implantable device of any of the preceding examples, but particularly example 155, wherein: each of the two or more first end anchors comprise a first stent portion with an outer diameter configured to substantially form a seal with an inner wall of the blood vessel; each of the two or more intermediate anchors comprise a spring element with a spring portion having an outer diameter configured to substantially form a seal with the inner wall of the blood vessel; and the two or more second end anchors comprise a second stent portion with an outer diameter configured to substantially form a seal with the inner wall of the blood vessel.

Example 161. The implantable device of any of the preceding examples, but particularly example 155, wherein: each of the two or more first end anchors comprise a first stent portion with an outer diameter configured to substantially form a seal with an inner wall of the blood vessel; each of the two or more intermediate anchors comprise a second stent portion with an outer diameter configured to substantially form a seal with the inner wall of the blood vessel; and the two or more second end anchors comprise a third stent portion with an outer diameter configured to substantially form a seal with the inner wall of the blood vessel.

Example 162. The implantable device of any of the preceding examples, but particularly example 155, wherein: the two or more intermediate anchors are configured to attach to an inner wall of the blood vessel, and the plurality of frame wires is composed of a shape memory alloy configured to tension to partially collapse the blood vessel at the second annular member or release tension to expand the blood vessel at the second annular member.

Example 163. The implantable device of any of the preceding examples, but particularly example 155, further comprising: a sensor configured to detect a pressure in the blood vessel; a microprocessor electrically coupled to the sensor; and a power source electrically coupled to the mechanical actuator, the microprocessor, and the sensor.

Example 164. The implantable device of any of the preceding examples, but particularly example 163, wherein the sensor comprises one of: a strain gauge, a piezoelectric sensor, a capacitance sensor, or a vacuum pressure sensor.

Example 165. The implantable device of any of the preceding examples, but particularly example 163, wherein the power source comprises an induction coil.

Example 166. The implantable device of any of the preceding examples, but particularly example 163, wherein the microprocessor is configured to: receive a signal from the sensor, the signal being indicative of the pressure in the blood vessel; and output a control signal to the mechanical actuator to either tension the plurality of frame wires or release tension in the plurality of frame wires based on the sensed pressure in the blood vessel.

Example 167. The implantable device of any of the preceding examples, but particularly example 155, wherein the tensioned plurality of frame wires is configured to restrict blood flow through the blood vessel by partially occluding the blood vessel.

Example 168. The implantable device of any of the preceding examples, but particularly example 155, wherein the blood vessel comprises a superior vena cava or an inferior vena cava.

Example 169. An implantable device for modulating blood flow through a blood vessel, the implantable device comprising: an expandable frame comprising a proximal end and a distal end and a longitudinal axis extending therethrough; and a membrane comprising an inflow end and an outflow end, wherein the inflow end is at least partially installed within the distal end of the expandable frame, wherein the membrane is configured to radially collapse at the outflow end and toward a central axis of the expandable frame, or radially expand away from the central axis of the expandable frame, in response to an actuation of a control wire coupled to a portion of the membrane, the central axis of the expandable frame being substantially parallel to the longitudinal axis.

Example 170. The implantable device of any of the preceding examples, but particularly example 169, further comprising a plurality of elongate support members coupled to the outflow end of the membrane, arranged radially around an outer surface of the membrane, and extending substantially parallel to the longitudinal axis, wherein the plurality of elongate support members is flexible to allow the membrane to bend radially toward the central axis of the frame at the outflow end when the control wire is actuated.

Example 171. The implantable device of any of the preceding examples, but particularly example 169, wherein the membrane is further configured to collapse at the outflow end in an asymmetrical collapse toward one or more portions of a circumferential edge of the outflow end.

Example 172. The implantable device of any of the preceding examples, but particularly example 169, wherein actuating the control wire results in configuring the membrane in an unrestricted blood flow state or a restricted blood flow state, wherein: the unrestricted blood flow state corresponds to the membrane radially expand away from the central axis of the expandable frame to allow blood flow through the blood vessel; and the restricted blood flow state corresponds to the membrane radially collapsing toward the central axis of the expandable frame to reduce blood flow through the blood vessel.

Example 173. The implantable device of any of the preceding examples, but particularly example 169, wherein the membrane is adjustable to a plurality of positions between expanded and collapsed, the plurality of positions including at least: an expanded position configured to allow the blood flow through the blood vessel; a partially expanded position configured to partially occlude the blood vessel; and a collapsed position configured to block the outflow end to occlude the blood vessel.

Example 174. The implantable device of any of the preceding examples, but particularly example 173, further comprising: a plurality of elongate support members coupled to the outflow end of the membrane, arranged radially around an outer surface of the membrane; and a plurality of eyelets, wherein each respective eyelet of the plurality of eyelets is coupled to a distal end of the expandable frame, each eyelet being configured to receive a portion of the control wire threaded therethrough such that the actuation of the control wire reversibly cinches the membrane by bringing the plurality of eyelets together at the outflow end to occlude or partially occlude the blood vessel.

Example 175. The implantable device of any of the preceding examples, but particularly example 174, wherein each of the plurality of eyelets comprises an aperture, and wherein the aperture of each respective eyelet in the plurality of eyelets is arranged to receive the control wire when threaded therethrough such that when the control wire is actuated, the plurality of eyelets move radially toward the central axis of the expandable frame.

Example 176. The implantable device of any of the preceding examples, but particularly example 169, further comprising a skirt membrane wrapped around at least a portion of an exterior surface of the expandable frame.

Example 177. The implantable device of any of the preceding examples, but particularly example 169, further comprising a power source coupled to the control wire, the power source comprising a battery or a wall outlet.

Example 178. The implantable device of any of the preceding examples, but particularly example 169, further comprising: an actuation device for actuating the control wire of the implantable device, the actuation device comprising: an actuator coupled to the control wire of the implantable device, and a first magnet configured to induce rotation of the actuator; and a control device communicatively coupled to the actuator, wherein the control device is implanted and comprises a second magnet configured to generate a changing magnetic field pole direction to cause rotation of the first magnet, wherein the actuation device is configured to send a first signal to the control wire to activate application of tension to the control wire and to send a second signal to the control wire to activate release of the tension from the control wire.

Example 179. The implantable device of any of the preceding examples, but particularly example 178, wherein the actuation device is further configured to: cause rotation of the second magnet in a first direction to induce rotation of the first magnet, thereby causing the actuator to tension the control wire to cause the membrane to radially collapse inward at the outflow end; and cause rotation of the second magnet in a second direction to induce rotation of the first magnet, thereby causing the actuator to release tension in the control wire to cause the membrane to radially open at the outflow end.

Example 180. The implantable device of any of the preceding examples, but particularly example 169, wherein the implantable device is configured to modulate a volume of blood flowing from a vena cava into a right atrium to decrease right atrial pressure.

Example 181. A device for modulating blood flow through a blood vessel, the device being configured to be at least partially circumferentially disposed about an outer wall of the blood vessel, comprising: a ring having a control element circumferentially disposed in the ring, wherein the control element has a first looped end and a second free end that passes through the first looped end, and wherein the control element is configured to translate relative to an inner diameter of the ring; and a mechanical actuator configured to be coupled to the second free end of the control element, wherein the mechanical actuator is configured to: apply tension to the second free end to reduce a diameter of the control element disposed in the ring; and reduce tension on the second free end to maintain or increase the diameter of the control clement disposed in the ring.

Example 182. The device of any of the preceding examples, but particularly example 181, wherein the ring is a polymeric ring that comprises one or more of: silicone, polyurethane, polyacrylate, or polyethylene vinyl acetate.

Example 183. The device of any of the preceding examples, but particularly example 181, wherein the control element comprises a wire or a suture.

Example 184. The device of any of the preceding examples, but particularly example 181, wherein the mechanical actuator comprises a catheter, and wherein the control element is axially translatable within a lumen of the catheter to tension or release tension in the control element.

Example 185. The device of any of the preceding examples, but particularly example 181, wherein the mechanical actuator comprises a linear actuator comprising: a pneumatic linear actuator, an electromechanical linear actuator, or a hydraulic linear actuator.

Example 186. The device of any of the preceding examples, but particularly example 181, further comprising: a sensor configured to detect a pressure in the blood vessel, the sensor comprising one of: a strain gauge, a piezoelectric sensor, a capacitance sensor, or a vacuum pressure sensor; a microprocessor electrically coupled to the sensor; and a power source electrically coupled to the mechanical actuator, the microprocessor, and the sensor.

Example 187. The device of any of the preceding examples, but particularly example 186, wherein the microprocessor is configured to: receive a signal from the sensor, the signal being indicative of the pressure in the blood vessel; and output a control signal to the mechanical actuator to either tension the control element or release tension in the control element based on the received pressure in the blood vessel.

Example 188. The device of any of the preceding examples, but particularly example 181, wherein the blood vessel comprises a superior vena cava or an inferior vena cava.

The spatially relative terms “outer,” “inner,” “upper,” “lower,” “below,” “above,” “vertical,” “horizontal,” and similar terms, may be used herein for ease of description to describe the relations between one element or component and another clement or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the control device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “projection” may include, and is contemplated to include, a plurality of projections. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable device for modulating blood flow through a blood vessel, the implantable device comprising:
an expandable frame comprising a proximal end and a distal end and a longitudinal axis extending therethrough;
a membrane comprising an inflow end and an outflow end, wherein the inflow end is at least partially installed within the distal end of the expandable frame;
an actuator coupled to a control wire, the control wire being coupled to a portion of the membrane;
a first magnet configured to induce rotation of the actuator to actuate the control wire to activate application of tension to the control wire and cause the membrane to radially collapse at the outflow end; and
a second magnet configured to generate a changing magnetic field pole direction to cause rotation of the first magnet to cause the membrane to radially expand at the outflow end.

2. The implantable device of claim 1, further comprising a plurality of elongate support members coupled to the outflow end of the membrane, arranged radially around an outer surface of the membrane, and extending substantially parallel to the longitudinal axis, wherein the plurality of elongate support members is flexible to allow the membrane to bend radially toward a central axis of the expandable frame at the outflow end when the control wire is actuated.

3. The implantable device of claim 1, wherein the membrane is further configured to collapse at the outflow end in an asymmetrical collapse toward one or more portions of a circumferential edge of the outflow end.

4. The implantable device of claim 1, wherein actuating the control wire results in configuring the membrane in an unrestricted blood flow state or a restricted blood flow state, wherein:
the unrestricted blood flow state corresponds to the membrane radially expand away from a central axis of the expandable frame to allow blood flow through the blood vessel; and
the restricted blood flow state corresponds to the membrane radially collapsing toward the central axis of the expandable frame to reduce blood flow through the blood vessel.

5. The implantable device of claim 1, wherein the membrane is adjustable to a plurality of positions between expanded and collapsed, the plurality of positions including at least:
an expanded position configured to allow the blood flow through the blood vessel;
a partially expanded position configured to partially occlude the blood vessel; and
a collapsed position configured to block the outflow end to occlude the blood vessel.

6. The implantable device of claim 5, further comprising:
a plurality of elongate support members coupled to the outflow end of the membrane, arranged radially around an outer surface of the membrane; and
a plurality of eyelets, wherein each respective eyelet of the plurality of eyelets is coupled to a distal end of the expandable frame, each eyelet being configured to receive a portion of the control wire threaded therethrough such that the actuation of the control wire reversibly cinches the membrane by bringing the plurality of eyelets together at the outflow end to occlude or partially occlude the blood vessel.

7. The implantable device of claim 6, wherein each of the plurality of eyelets comprises an aperture, and wherein the aperture of each respective eyelet in the plurality of eyelets is arranged to receive the control wire when threaded therethrough such that when the control wire is actuated, the plurality of eyelets move radially toward a central axis of the expandable frame.

8. The implantable device of claim 1, further comprising a skirt membrane wrapped around at least a portion of an exterior surface of the expandable frame.

9. The implantable device of claim 1, further comprising a power source coupled to the control wire, the power source comprising a battery or a wall outlet.

10. The implantable device of claim 1, wherein the actuation of the control wire, the actuation device is further configured to:
cause rotation of the second magnet in a first direction to induce rotation of the first magnet, thereby causing the actuator to tension the control wire to cause the membrane to radially collapse inward at the outflow end; and
cause rotation of the second magnet in a second direction to induce rotation of the first magnet, thereby causing the actuator to release tension in the control wire to cause the membrane to radially open at the outflow end.

11. The implantable device of claim 1, wherein the implantable device is configured to modulate a volume of blood flowing from a vena cava into a right atrium to decrease right atrial pressure.

12. The implantable device of claim 1, wherein the blood vessel comprises a superior vena cava or an inferior vena cava.

13. An implantable device for modulating blood flow through a blood vessel, the implantable device comprising:
an expandable frame comprising a proximal end and a distal end;
a membrane comprising an inflow end and an outflow end, wherein the inflow end is at least partially installed within the distal end of the expandable frame;
an actuator coupled to a control wire, the control wire being coupled to a portion of the membrane;
a first magnet configured to induce rotation of the actuator; and
an implantable control device communicatively coupled to the actuator and comprising a second magnet configured to generate a changing magnetic field pole direction to cause rotation of the first magnet.

14. The implantable device of claim 13, wherein the membrane is configured to radially collapse at the outflow end in response to receiving a first signal at the actuator to actuate the control wire to activate application of tension to the control wire, or radially expand, in response to receiving a second signal at the actuator to actuate the control wire to activate release of the tension from the control wire.

15. The implantable device of claim 13, wherein the membrane is adjustable to a plurality of positions between expanded and collapsed, the plurality of positions including at least:
an expanded position configured to allow the blood flow through the blood vessel;
a partially expanded position configured to partially occlude the blood vessel; and
a collapsed position configured to block the outflow end to occlude the blood vessel.

16. The implantable device of claim 13, wherein actuation of the control wire is further configured to:
cause rotation of the second magnet in a first direction to induce rotation of the first magnet, thereby causing the actuator to tension the control wire to cause the membrane to radially collapse inward at the outflow end; and
cause rotation of the second magnet in a second direction to induce rotation of the first magnet, thereby causing the actuator to release tension in the control wire to cause the membrane to radially open at the outflow end.

17. The implantable device of claim 13, wherein the implantable device is configured to modulate a volume of blood flowing from a vena cava into a right atrium to decrease right atrial pressure.

18. An implantable device for modulating blood flow through a blood vessel, the implantable device comprising:
an expandable frame comprising a proximal end and a distal end; and
a membrane comprising an inflow end and an outflow end, wherein the inflow end is at least partially installed within the distal end of the expandable frame
a magnet configured to induce rotation of an actuator coupled to a control wire; and
wherein actuation of the control wire induces rotation of the magnet to cause the actuator to tension or release the control wire to actuate the membrane.

19. The implantable device of claim 18, wherein the membrane is adjustable to a plurality of positions between expanded and collapsed, the plurality of positions including at least:
an expanded position configured to allow the blood flow through the blood vessel;
a partially expanded position configured to partially occlude the blood vessel; and
a collapsed position configured to block the outflow end to occlude the blood vessel.

20. The implantable device of claim 18, wherein the implantable device is configured to modulate a volume of blood flowing from a vena cava into a right atrium to decrease right atrial pressure.
